(19) United States
(12) Patent Application Publication
(10) Pub. No.: US 2026/0232672 A1
(43) Pub. Date: Aug. 13, 2026

(54) COMPOSITIONS AND METHODS OF TREATING FEMALE SEXUAL DYSFUNCTION AND FEMALE SEXUAL AROUSAL DISORDER

(71) Applicant: Daré Bioscience, Inc., San Diego, CA (US)

(72) Inventor: David Friend, San Diego, CA (US)

(21) Appl. No.: 19/156,203

(22) PCT Filed: Feb. 14, 2024
(86) PCT No.: PCT/US24/15721

§ 371 Date: Aug. 13, 2025

Related U.S. Application Data
(60) Provisional application No. 63/484,812, filed on Feb. 14, 2023.
(60) Provisional application No. 63/506,302, filed on Jun. 5, 2023.
(60) Provisional application No. 63/513,082, filed on Jul. 11, 2023.
(60) Provisional application No. 63/595,279, filed on Nov. 1, 2023.

(51) Int. Cl. A61K 31/519; A61K 9/00; A61P 15/00

(52) U.S. Cl. CPC A61K 31/519; A61K 9/0034; A61P 15/00

(57) ABSTRACT

The present disclosure generally relates to compositions and techniques for treatment of female sexual dysfunction (FSD), including female sexual arousal disorder (FSAD). In some embodiments, a composition such as a cream may be applied to the genital of a subject. The composition may also contain an active ingredient for treatment of FSD and/or FSAD, e.g., sildenafil, and/or pharmaceutically acceptable salts thereof. In addition, certain embodiments as described herein are generally directed to techniques for using such compositions, kits including such compositions, or the like.

### RELATED APPLICATIONS

This application is a National Stage Application, filed under 35 U.S.C. § 371, of International Application No. PCT/US2024/015721 filed Feb. 14, 2024, which claims the benefit of priority under 35 U.S.C. § 119 (e) to U.S. Provisional Application No. 63/484,812, filed Feb. 14, 2023, U.S. Provisional Application No. 63/506,302, filed Jun. 5, 2023, and U.S. Provisional Application No. 63/513,082, filed Jul. 11, 2023, U.S. Provisional Application No. 63/595,279, filed Nov. 1, 2023, the entire contents of which is incorporated herein by reference in its entirety.

### FIELD

The present disclosure generally relates to compositions and methods for treatment of female sexual dysfunction (FSD), e.g., female sexual arousal disorder (FSAD), and symptoms thereof.

### BACKGROUND

Female Sexual Dysfunction (FSD) is a recognized problem that significantly impacts quality of life. One type of FSD is called Female Sexual Arousal Disorder (FSAD) and is estimated to currently affect approximately 20% of women in the United States. Currently, patients with FSAD are usually treated with counselling. Another type of FSD is Female Sexual Interest/Arousal Disorder (FSIAD). FSIAD is defined in the DSM-5 as lack of, or significantly reduced, sexual interest/arousal, as measured by the presence of three of the following six symptoms: absent or reduced interest in sexual activity; absent or reduced sexual thoughts or fantasies; no or reduced initiation of sexual activity, and typically unreceptive to a partner's attempts to initiate; absent or reduced sexual excitement or pleasure in almost all or all sexual encounters; absent or reduced sexual interest/arousal in response to any internal or external sexual cues; and absent or reduced genital or non-genital sensations during sexual activity in all or almost all sexual encounters. These symptoms must cause clinically significant distress and have persisted for a minimum of six months.

Phosphodiesterase type 5 inhibitors are drugs used to block the degradative action of phosphodiesterase type 5 on cyclic GMP in the smooth muscle cells lining the blood vessels supplying the corpus cavernosum of the penis. These drugs are commonly used in the treatment of erectile dysfunction. Phosphodiesterase type 5 inhibitors are commonly delivered orally and there are no transdermal formulations presently approved by the FDA. Previous studies have shown that when oral phosphodiesterase type 5 inhibitors are given daily for pulmonary hypertension, the most common treatment emergent adverse events (TEAEs) are similar to when phosphodiesterase type 5 inhibitors are given orally on an as needed basis for erectile disfunction (ED) (headache, flushing). As pulmonary hypertension is more common in women compared to men, the FDA safety data for daily oral phosphodiesterase 5 inhibitor use are primarily (75-78%) among women, who have severe cardiac and respiratory comorbidities due to their underlying pulmonary hypertension diagnosis. Thus, women using substantially larger daily systemic doses of oral phosphodiesterase 5 inhibitors for pulmonary hypertension do not experience more severe adverse events as compared to the use of phosphodiesterase 5 inhibitors for ED. There are no FDA-approved treatments for FSD, FSAD, or FSIAD. Accordingly, systems and methods for transdermally delivering clinically useful amounts of phosphodiesterase type 5 inhibitors and improved treatment methods for FSD, including FSAD and FSIAD, and symptoms thereof are needed.

### SUMMARY

The present disclosure generally relates to compositions and methods for treating Female Sexual Dysfunction (FSD) or symptom thereof, comprising applying to a genital area a composition comprising a phosphodiesterase type 5 inhibitor and/or a salt thereof. The present disclosure further relates to compositions and methods for treating Female Sexual Arousal Disorder (FSAD) or symptom thereof, comprising applying to a genital area a composition comprising a phosphodiesterase type 5 inhibitor and/or a salt thereof. The present disclosure further relates to compositions and methods for treating Female Sexual Interest/Arousal Disorder (FSIAD) or symptom thereof, comprising applying to a genital area a composition comprising a phosphodiesterase type 5 inhibitor and/or a salt thereof. In embodiments, the composition further includes a stabilization polymer, propylene glycol, a polysorbate surfactant, a nitric oxide donor, and an ionic salt. In embodiments, the composition further includes a stabilization polymer, propylene glycol, a polysorbate surfactant, and an ionic salt. In embodiments, the composition further includes a humectant. In embodiments, the composition further includes L-arginine or L-arginine salt. In embodiments where L-arginine or L-arginine salt is recited, any humectant may be substituted for L-arginine or L-arginine salt. In some embodiments, the composition is a cream. In some embodiments, the composition is a gel. In some embodiments, the composition is a lotion.

In some embodiments, treating FSAD includes increasing satisfactory sexual events, improving arousal sensation, increasing arousal lubrication, increasing orgasm, decreasing concern with sexual arousal, decreasing pain or discomfort during and/or after sexual activity, and/or improving satisfaction with sexual activity. In some embodiments, treating FSAD includes improving arousal sensation. In some embodiments, treating FSAD includes increasing arousal lubrication. In some embodiments, treating FSAD includes increasing orgasm. In some embodiments, treating FSAD includes decreasing concern with sexual arousal. In some embodiments, treating FSAD includes improving satisfaction with sexual activity. In some embodiments, treating FASD includes decreasing pain or discomfort during and/or after sexual activity.

In some embodiments, treating FSAD includes increasing satisfactory sexual events. In embodiments, increasing satisfactory sexual events includes, but is not limited to, increasing orgasm or orgasm frequency, feeling close to or connection with a sexual partner, experiencing pleasurable sensations, increased physical sensation, feelings of arousal, feelings of passion, and/or having an enjoyable sexual experience.

In some embodiments, treating FSAD includes increasing sensation at the genital area. In some embodiments, sensation at the genital are includes one or more of: tingling, pleasure, warmth, pulsating, throbbing, engorgement, or fullness at the genital area.

In some aspects, described herein is a method for increasing satisfactory sexual events in a female subject during sexual activity, comprising applying to a genital area of a female subject a topical composition comprising a phosphodiesterase type 5 inhibitor and/or a salt thereof.

In some aspects, described herein is a method for improving arousal sensation in a female subject during sexual activity, comprising applying to a genital area of a female subject a topical composition comprising a phosphodiesterase type 5 inhibitor and/or a salt thereof.

In some aspects, described herein is a method for increasing arousal lubrication in a female subject during sexual activity, comprising applying to a genital area of a female subject a topical composition comprising a phosphodiesterase type 5 inhibitor and/or a salt thereof.

In some aspects, described herein is a method for increasing orgasm in a female subject during sexual activity, comprising applying to a genital area of a female subject a topical composition comprising a phosphodiesterase type 5 inhibitor and/or a salt thereof.

In some aspects, described herein is a method for decreasing concern with sexual arousal or improving satisfaction with sexual activity in a female subject, comprising applying to a genital area of a female subject a topical composition comprising a phosphodiesterase type 5 inhibitor and/or a salt thereof.

In some aspects, described herein is a method for decreasing pain or discomfort during and/or after sexual activity in a female subject, comprising applying to a genital area of a female subject a topical composition comprising a phosphodiesterase type 5 inhibitor and/or a salt thereof.

In some aspects, described herein is a method for treating Female Sexual Dysfunction (FSD) or symptom thereof, comprising applying to a genital area of a female subject a topical composition comprising a phosphodiesterase type 5 inhibitor and/or a salt thereof.

In some aspects, described herein is a method for treating Female Sexual Interest/Arousal Disorder (FSIAD) or symptom thereof, comprising applying to a genital area of a female subject a topical composition comprising a phosphodiesterase type 5 inhibitor and/or a salt thereof. In embodiments, treating FSIAD comprises increasing sexual desire. In embodiments, treating FSIAD comprises increasing response to physical or mental sexual stimulation. In embodiments, the symptom of FSIAD comprises absent or reduced interest in sexual activity. In embodiments, the symptom of FSIAD comprises absent or reduced erotic thoughts or fantasies. In embodiments, the symptom of FSIAD comprises reduced sexual desire. In embodiments, the symptom of FSIAD comprises reduced response to physical sexual stimulation. In embodiments, the symptom of FSIAD comprises reduced response to mental sexual stimulation.

In some aspects, described herein is a method for increasing sexual desire in a female subject, comprising applying to a genital area of the female subject a topical composition comprising a phosphodiesterase type 5 inhibitor and/or a salt thereof.

In some aspects, described herein is a method for increasing response to physical or mental sexual stimulation in a female subject, comprising applying to a genital area of the female subject a topical composition comprising a phosphodiesterase type 5 inhibitor and/or a salt thereof. In embodiments, response to physical sexual stimulation is increased. In embodiments, response to mental sexual stimulation is increased. In embodiments, response to physical and mental sexual stimulation is increased.

In embodiments, the phosphodiesterase type 5 inhibitor and/or salt thereof is administered at least twice over a period of time. In embodiments, the period of time is about 1 week to about 8 weeks.

In embodiments, one or more symptoms is improved within about 1 week to about 8 weeks of an initial administration. In embodiments, one or more symptoms is improved within about 1 week to about 4 weeks of the initial administration. In embodiments, the one or more symptoms comprises arousal lubrication. In embodiments, the one or more symptoms comprises sexual desire. In embodiments, the one or more symptoms comprises achieving and/or pleasure of orgasm.

In some embodiments, the phosphodiesterase type 5 inhibitor is sildenafil, avanafil, lodenafil, mirodenafil, tadalafil, vardenafil, udenafil, acetildenafil, or thiomethisosildenafil. In some embodiments, the phosphodiesterase type 5 inhibitor is sildenafil.

In some embodiments, the composition comprises an L-arginine salt. In some embodiments, the L-arginine salt comprises L-arginine HCl. In some embodiments, the L-arginine or L-arginine salt is present at a concentration of at least about 5% by weight of the composition. In some embodiments, the L-arginine or L-arginine salt is present at a concentration of at least about 7% by weight of the composition. In some embodiments, the ionic salt is capable of driving the phosphodiesterase type 5 inhibitor and/or salt thereof through a subject's stratum corneum. In some embodiments, the ionic salt is present at a concentration of at least about 5% by weight of the composition. In some embodiments, the ionic salt comprises potassium chloride. In some embodiments, the stabilization polymer comprises xanthan gum. In some embodiments, the stabilization polymer is present at a concentration of at least about 0.8% by weight of the composition. In some embodiments, the propylene glycol is present at a concentration of at least about 8% by weight of the composition. In some embodiments, the polysorbate surfactant comprises Polysorbate 20. In some embodiments, the polysorbate surfactant is present at a concentration of at least about 2% by weight of the composition.

In some embodiments, the composition comprises a salt of a phosphodiesterase type 5 inhibitor. In some embodiments, the composition comprises a sodium salt of a phosphodiesterase type 5 inhibitor. In some embodiments, the composition comprises a citrate salt of a phosphodiesterase type 5 inhibitor. In some embodiments, the phosphodiesterase type 5 inhibitor is sildenafil. In some embodiments, the phosphodiesterase type 5 inhibitor and/or salt thereof is present at a concentration of at least about 1% by weight of the composition. In some embodiments, the phosphodiesterase type 5 inhibitor and/or salt thereof is present at a concentration of at least about 5% by weight of the composition. In some embodiments, the phosphodiesterase type 5 inhibitor and/or salt thereof is present at a concentration of at least about 7% by weight of the composition. In some embodiments, the phosphodiesterase type 5 inhibitor and/or salt thereof is present at a concentration of between about 1% and about 10% by weight of the composition.

In some embodiments, the composition comprises glyceryl stearate. In some embodiments, the composition comprises cetyl alcohol. In some embodiments, the composition comprises squalane. In some embodiments, the composition comprises isopropyl myristate. In some embodiments, the composition comprises oleic acid. In some embodiments, the composition comprises trisodium citrate dihydrate. In some embodiments, the composition comprises sodium benzoate. In some embodiments, the composition comprises gluconolactone. In some embodiments, purified water is present at a concentration of at least about 40% by weight of the composition. In some embodiments, the subject is human.

The present disclosure generally relates to a method for treating Female Sexual Arousal Disorder (FSAD) or symptom thereof, comprising applying a composition for topical delivery to a genital area of a subject, wherein at least about 80% by weight of the composition comprises purified water, at least one chloride salt, a stabilization polymer, propylene glycol, a polysorbate surfactant, L-arginine or L-arginine salt, and a phosphodiesterase type 5 inhibitor and/or a salt thereof.

The present disclosure generally relates to a method for treating Female Sexual Arousal Disorder (FSAD) or symptom thereof, comprising applying to a genital area a composition comprising an ionic salt, a phosphodiesterase type 5 inhibitor and/or a salt thereof, and optionally, L-arginine or L-arginine salt.

The present disclosure generally relates to a method for treating Female Sexual Arousal Disorder (FSAD) or symptom thereof, comprising applying to a genital area a composition consisting essentially of purified water, potassium chloride, L-Arginine HCl, glyceryl stearate, cetyl alcohol, squalene, xanthan gum, isopropyl myristate, oleic acid, propylene glycol, polysorbate 20, and sildenafil citrate.

The present disclosure generally relates to a method for treating Female Sexual Arousal Disorder (FSAD) or symptom thereof, comprising applying to a genital area a composition comprising each of the following compounds at concentrations of no more than +20% of the stated concentrations:
a. purified water at a concentration of about 35% to about 55% by weight;
b. potassium chloride at a concentration of about 2.5% to about 15% by weight;
c. L-Arginine HCl at a concentration of about 2.5% to about 15% by weight;
d. glyceryl stearate at a concentration of about 4% to about 10% by weight;
e. cetyl alcohol at a concentration of about 4% to about 10% by weight;
f. squalane at a concentration of about 1% to about 8% by weight;
g. xanthan gum at a concentration of about 0.1% to about 5% by weight;
h. isopropyl myristate at a concentration of about 0.1% to about 5% by weight;
i. oleic acid at a concentration of about 0.1% to about 5% by weight;
j. propylene glycol at a concentration of about 1% to about 10% by weight;
k. polysorbate 20 at a concentration of about 0.2% to about 5% by weight; and
l. sildenafil citrate at a concentration of about 1% to about 10% by weight.

In some embodiments, the composition comprises the compounds recited above at concentrations of no more than ±10% of the stated concentrations.

The present disclosure generally relates to a method for treating Female Sexual Arousal Disorder (FSAD) or symptom thereof, comprising applying to a subject, a composition comprising a phosphodiesterase type 5 inhibitor and/or a salt thereof, a stabilization polymer, propylene glycol, a polysorbate surfactant, L-arginine or L-arginine salt, and an ionic salt.

In some embodiments, the phosphodiesterase type 5 inhibitor comprises sildenafil or pharmaceutically acceptable salt thereof. In some embodiments, the subject has Female Sexual Arousal Disorder (FSAD). In some embodiments, the subject is at risk of Female Sexual Arousal Disorder (FSAD). In some embodiments, the subject is human. In some embodiments, the composition is a cream. In some embodiments, the composition is applied to a clitoris, vestibule, vulva, and/or vagina of the subject. In some embodiments, the composition is applied to a clitoris, glans, and/or frenulum of a genital area of the subject. In some embodiments, the composition is applied to a clitoris, glans, frenulum, and/or vestibule of a genital area of the subject. In some embodiments, the composition is applied to a clitoris, glans, frenulum, vestibule, and/or labia minora of a genital area of the subject. In some embodiments, the composition is not applied to a labia majora of a genital area of the subject. In some embodiments, the composition is applied intravaginally to the anterior distal vagina of the subject.

In some embodiments, the composition comprises:
a. purified water at a concentration of about 40% by weight;
b. potassium chloride at a concentration of about 5% by weight;
c. L-Arginine HCl at a concentration of about 7.5% by weight;
d. glyceryl stearate at a concentration of about 7% by weight;
e. cetyl alcohol at a concentration of about 7% by weight;
f. squalane at a concentration of about 4% by weight;
g. xanthan gum at a concentration of about 0.8% by weight;
h. isopropyl myristate at a concentration of about 1% by weight;
i. oleic acid at a concentration of about 1% by weight;
j. propylene glycol at a concentration of about 8.5% by weight;
k. polysorbate 20 at a concentration of about 2% by weight;
l. trisodium citrate dihydrate at a concentration of about 10% by weight;
m. sodium benzoate at a concentration of about 0.2% by weight;
n. gluconolactone at a concentration of about 0.25% by weight; and
o. sildenafil citrate at a concentration of about 5% by weight.

The present disclosure generally relates to a method for treating Female Sexual Arousal Disorder (FSAD) or symptom thereof, comprising selecting a female subject exhibiting one or more symptom of FSAD, and administering to a genital area of the subject a composition comprising a phosphodiesterase type 5 inhibitor and/or a salt thereof. In embodiments, the composition further includes a stabilization polymer, propylene glycol, a polysorbate surfactant, a L-arginine or L-arginine salt, and an ionic salt. In some embodiments, the one or more symptoms are selected from decreased genital sensitivity, lack of genital responsiveness during sexual activity, diminished genital arousal, and/or distress regarding sexual arousal difficulties. In embodiments, the one or more symptoms includes reduced response to physical sexual stimulation. In embodiments, the female subject's primary complaint is reduced response to physical sexual stimulation.

The present disclosure generally relates to a method for treating Female Sexual Interest/Arousal Disorder (FSIAD) or symptom thereof, comprising: selecting a female subject exhibiting one or more symptom of FSIAD, and administering to a genital area of the subject a composition comprising a phosphodiesterase type 5 inhibitor and/or a salt thereof, a stabilization polymer, propylene glycol, a polysorbate surfactant, a L-arginine or L-arginine salt, and an ionic salt. In embodiments, the one or more symptoms of FSIAD are selected from absent or reduced interest in sexual activity or erotic thoughts or fantasies, reduced sexual desire, and/or reduced response to physical or mental sexual stimulation. In embodiments, the one or more symptoms of FSIAD comprises absent or reduced interest in sexual activity. In embodiments, the one or more symptoms of FSIAD comprises absent or reduced erotic thoughts or fantasies. In embodiments, the one or more symptoms of FSIAD comprises reduced sexual desire. In embodiments, the one or more symptoms of FSIAD comprises reduced response to physical sexual stimulation. In embodiments, the one or more symptoms of FSIAD comprises reduced response to mental sexual stimulation. In embodiments, the female subject's primary complaint is reduced response to physical sexual stimulation.

In some embodiments, about 50% of the composition is applied externally to the vulva and about 50% of the composition is applied intravaginally. In some embodiments, administering about 50% of the composition externally comprises applying the composition at the anterior labial commissure, prepuce of the clitoris, glans, and frenulum; applying the composition to the vestibule of the vagina; and applying the composition to the labia minora. In some embodiments, the composition is not applied to the labia majora or pubic hair. In some embodiments, administering about 50% of the composition intravaginally comprises: applying the composition to the anterior distal vagina at a depth of between about 0 cm and about 3 cm in the vagina. In some embodiments, administering about 50% of the composition intravaginally comprises: applying the composition about halfway between the distal and proximal interphalangeal joints, on the distal, lower ⅓ of the vagina.

In some embodiments, the composition is administered between about 10 to about 20 minutes prior to a sexual activity. In some embodiments, the composition is administered at most about nine doses in about 4 weeks. In some embodiments, the composition is administered at least about 24 hours before a second dose of the composition. In some embodiments, the subject has not been administered at least one of guanylate cyclase stimulators, clonidine, CYP3A4 inhibitors, nitric oxide donors, organic nitrates, organic nitrites, or alpha blockers within 28 days prior to the administration of a first dose of the composition.

In some embodiments, the subject avoids wiping or washing off any of the genital area where the composition has been applied.

In some embodiments, after sexual activity has ended, any remaining composition is washed off.

In some embodiments, administering to a genital area of the subject a composition comprising a phosphodiesterase type 5 inhibitor and/or a salt thereof results in increased sexual arousal.

In some embodiments, administering to a genital area of the subject a composition comprising a phosphodiesterase type 5 inhibitor and/or a salt thereof results in increased sexual excitement.

In some embodiments, administering to a genital area of the subject a composition comprising a phosphodiesterase type 5 inhibitor and/or a salt thereof results in a satisfactory sexual event.

In some embodiments, administering to a genital area of the subject a composition comprising a phosphodiesterase type 5 inhibitor and/or a salt thereof results in increased genital arousal sensations.

In some embodiments, administering to a genital area of the subject a composition comprising a phosphodiesterase type 5 inhibitor and/or a salt thereof results in increased lubrication of the genital area during sexual activity.

In some embodiments, administering to a genital area of the subject a composition comprising a phosphodiesterase type 5 inhibitor and/or a salt thereof results in increased cognitive sexual arousal.

In some embodiments, administering to a genital area of the subject a composition comprising a phosphodiesterase type 5 inhibitor and/or a salt thereof results in increased sexual desire.

In some embodiments, administering to a genital area of the subject a composition comprising a phosphodiesterase type 5 inhibitor and/or a salt thereof results in increased orgasm during sexual activity.

In some embodiments, administering to a genital area of the subject a composition comprising a phosphodiesterase type 5 inhibitor and/or a salt thereof results in decreased difficulty or inability to orgasm during sexual activity.

In some embodiments, administering to a genital area of the subject a composition comprising a phosphodiesterase type 5 inhibitor and/or a salt thereof results in increased feeling or sensation of the genital area during sexual activity.

In some embodiments, administering to a genital area of the subject a composition comprising a phosphodiesterase type 5 inhibitor and/or a salt thereof results in increased tingling of the genital area during sexual activity.

In some embodiments, administering to a genital area of the subject a composition comprising a phosphodiesterase type 5 inhibitor and/or a salt thereof results in increased engorgement of the genital area during sexual activity.

In some embodiments, administering to a genital area of the subject a composition comprising a phosphodiesterase type 5 inhibitor and/or a salt thereof results in increased warmth, throbbing, or pulsating of the genital area during sexual activity.

In some embodiments, administering to a genital area of the subject a composition comprising a phosphodiesterase type 5 inhibitor and/or a salt thereof results in decreased pain and/or discomfort during sexual activity.

In some embodiments, administering to a genital area of the subject a composition comprising a phosphodiesterase type 5 inhibitor and/or a salt thereof results in decreased pain and/or discomfort after sexual activity.

In some embodiments, administering to a genital area of the subject a composition comprising a phosphodiesterase type 5 inhibitor and/or a salt thereof results in increased sexual excitement.

In some embodiments, one or more symptoms of Female Sexual Arousal Disorder (FSAD) includes, but is not limited to, reduced response to physical sexual stimulation.

In some embodiments, the female subject's primary complaint is reduced response to physical sexual stimulation.

In some embodiments, a subject exhibits one or more of the following after administration of a composition comprising a phosphodiesterase type 5 inhibitor and/or a salt
a. improvement in Sexual Function Questionnaire (SFQ28) arousal cognitive domain score of at least 1;
b. improvement in SFQ28 arousal lubrication domain score of at least 1;
c. improvement in SFQ28 arousal sensation domain score of at least 1.5;
d. improvement in SFQ28 desire domain score of at least 2;
e. improvement in SFQ28 orgasm domain score of at least 1.20;
f. improvement in SFQ28 pain domain score of at least 1;
g. improvement in Female Sexual Distress Scale-Desire, Arousal, Orgasm (FSDS-DAO) score of at least-7;
h. improvement in an arousal sensation score of at least 1.45;
i. improvement in genital arousal score of at least 1.13;
j. improvement in genital arousal concerns score of at least 1.49;
k. improvement in Arousal Diary Item 12-Proportion of SSEs of at least 0.25; and
l. improvement in Arousal Diary Item 12-Number of SSEs of at least 1.41.

In some embodiments, the subject exhibits 2 or more of a-l. In some embodiments, the subject exhibits 3 or more of a-l. In some embodiments, the subject exhibits 4 or more of a-l. In some embodiments, the subject exhibits 5 or more of a-l. In some embodiments, the subject exhibits 6 or more of a-l. In some embodiments, the subject exhibits 7 or more of a-l. In some embodiments, the subject exhibits 8 or more of a-l. In some embodiments, the subject exhibits 9 or more of a-l. In some embodiments, the subject exhibits 10 or more of a-l. In some embodiments, the subject exhibits 11 or more of a-l. In some embodiments, the subject exhibits all 12 of a-l.

In some embodiments, a subject comprises a primary diagnosis of FSAD as defined by the Diagnostic and Statistical Manual of Mental Disorders Text Revision Fourth Edition (DSM-IV-TR).

In some embodiments, a topical composition comprising a phosphodiesterase type 5 inhibitor and/or a salt thereof does not result in one or more of orthostatic hypotension, headache, flushing, dyspepsia, light headedness, abnormal vision, nasal congestion, back pain, myalgia, nausea, dizziness, and rash.

In some embodiments, a subject's primary complaint is FSAD or one of more symptoms thereof.

In some embodiments, a subject's primary complaint is FSAD or one of more symptoms thereof and the subject exhibits secondary HSDD symptoms. In some embodiments, a subject's primary complaint is FSAD and the subject does not exhibit Female Orgasmic Disorder (FOD) symptoms.

The present disclosure generally relates to a method for treating Female Sexual Arousal Disorder (FSAD) or symptom thereof, comprising:
a. selecting a female subject having a primary complaint of FSAD, and
b. applying to a genital area of the subject a topical composition comprising a phosphodiesterase type 5 inhibitor and/or a salt thereof.

In some embodiments, the subject exhibits secondary HSDD symptoms. In some embodiments, a subject has FSAD or symptoms thereof as their sole diagnosis. In some embodiments, a subject has primary FSAD or symptoms thereof and secondary HSDD or symptoms thereof. In some embodiments, a premenopausal subject has FSAD or symptoms thereof as their sole diagnosis. In some embodiments, a premenopausal subject has primary FSAD or symptoms thereof and secondary HSDD or symptoms thereof.

The present disclosure generally relates to a method of determining efficacy of a FSAD treatment, comprising:
a) obtaining a baseline score for one or more of: a Sexual Function Questionnaire (SFQ28) arousal cognitive domain score, a SFQ28 arousal lubrication domain score, a SFQ28 arousal sensation domain score, a SFQ28 desire domain score, a SFQ28 orgasm domain score, a SFQ28 pain domain score, a Female Sexual Distress Scale-Desire, Arousal, Orgasm (FSDS-DAO) score, an arousal sensation score, a genital arousal score, genital arousal concerns, Arousal Diary Item 12-Proportion of SSEs, and Arousal Diary Item 12-Number of SSEs;
b) administering the FSAD treatment to a female subject; and
c) determining improvement in one or more of the scores in step a.

In some embodiments, presence of one or more of the following indicates the FSAD treatment is effective:
a. improvement in Sexual Function Questionnaire (SFQ28) arousal cognitive domain score of at least 1;
b improvement in SFQ28 arousal lubrication domain score of at least 1;
c. improvement in SFQ28 arousal sensation domain score of at least 1.5;
d. improvement in SFQ28 desire domain score of at least 2;
e. improvement in SFQ28 orgasm domain score of at least 1.20;
f. improvement in SFQ28 pain domain score of at least 1;
g. improvement in Female Sexual Distress Scale-Desire, Arousal, Orgasm (FSDS-DAO) score of at least-7;
h. improvement in arousal sensation of at least 1.45;
i. improvement in genital arousal of at least 1.13;
j. improvement in genital arousal concerns of at least 1.49;
k. improvement in Arousal Diary Item 12-Proportion of SSEs of at least 0.25; and
l. improvement in Arousal Diary Item 12-Number of SSEs of at least 1.41.

In some embodiments, presence of 2 or more of a-l indicates the FSAD treatment is effective. In some embodiments, presence of 3 or more of a-l indicates the FSAD treatment is effective. In some embodiments, presence of 4 or more of a-l indicates the FSAD treatment is effective. In some embodiments, presence of 5 or more of a-l indicates the FSAD treatment is effective. In some embodiments, presence of 6 or more of a-l indicates the FSAD treatment is effective. In some embodiments, presence of 7 or more of a-l indicates the FSAD treatment is effective. In some embodiments, presence of 8 or more of a-l indicates the FSAD treatment is effective. In some embodiments, presence of 9 or more of a-l indicates the FSAD treatment is effective. In some embodiments, presence of 10 or more of a-l indicates the FSAD treatment is effective. In some embodiments, presence of 11 or more of a-l indicates the FSAD treatment is effective. In some embodiments, presence of all 12 of a-l indicates the FSAD treatment is effective.

In some embodiments, the methods disclosed herein comprise applying one or more additional doses of the topical FSAD treatment composition to the subject. In some embodiments, one or more additional doses are applied if the FSAD treatment is determined to be effective.

Other advantages and novel features of the present disclosure will become apparent from the following detailed description of various non-limiting embodiments of the disclosure when considered in conjunction with the accompanying figures.

### DETAILED DESCRIPTION

Female sexual dysfunction (FSD) is estimated to affect approximately 40% of U.S. women at some point during their lives. The prevalence of FSD increases with age and is associated with vascular risk factors and menopause.

FSAD is defined in the DSM IV-TR as a persistent or recurrent inability to attain, or to maintain until completion of the sexual activity, an adequate lubrication-swelling response of sexual excitement that causes marked distress or interpersonal difficulty. FSAD is estimated to negatively impact approximately 20% of U.S. women In addition, disruptions in sexual arousal impact other aspects of sexual response. Orgasm is not possible without arousal and a lack of arousal commonly leads to a lack of desire because sexual activity is not enjoyable or reinforcing. Sexual pain disorders may be intricately linked to a lack of sufficient sexual arousal. Intercourse without lubrication can be painful, and repeated intercourse without arousal may cause vulvar infections, chronic irritation, and may lead to secondary vaginismus, fear of sex, or the avoidance of sexual activity altogether.

FSIAD is defined in the DSM-5 as lack of, or significantly reduced, sexual interest/arousal, as measured by the presence of three of the following six symptoms: absent or reduced interest in sexual activity; absent or reduced sexual thoughts or fantasies; no or reduced initiation of sexual activity, and typically unreceptive to a partner's attempts to initiate; absent or reduced sexual excitement or pleasure in almost all or all sexual encounters; absent or reduced sexual interest/arousal in response to any internal or external sexual cues; and absent or reduced genital or non-genital sensations during sexual activity in all or almost all sexual encounters. These symptoms must cause clinically significant distress and have persisted for a minimum of six months.

The present disclosure generally relates to compositions and techniques for the treatment of FSD, Female Sexual Arousal Disorder (FSAD) with the transdermal and/or transmucosal delivery of various compounds. In some embodiments, compositions and techniques of the present disclosure treat FSIAD. In some embodiments, a composition such as a cream may be applied to the genital area of a subject. The composition may contain an active ingredient, e.g., phosphodiesterase type 5 inhibitor, and/or salt thereof. In some embodiments, the active ingredient is sildenafil or salt thereof.

The present disclosure provides a topical formulation of sildenafil cream which unexpectedly offers several potential clinical advantages over oral administration of sildenafil citrate, including: more targeted local delivery at required therapeutic levels; reduced systemic exposure, thereby reducing known adverse events; and more rapid onset of action to achieve a more immediate therapeutic benefit.

One set of embodiments provides a composition for topical delivery comprising a phosphodiesterase type 5 inhibitor and/or a salt thereof, and optionally, an ionic salt and/or a L-arginine or L-arginine salt. In some embodiments, the composition may be stabilized using a combination of a stabilization polymer (e.g., xanthan gum, KELTROL® BT and/or KELTROL® RD), propylene glycol, and a polysorbate surfactant (e.g., Polysorbate 20), which in combination provides stability to the composition as compared to compositions lacking one or more of these.

In one set of embodiments, the pharmaceutical agent is a phosphodiesterase type 5 inhibitor and/or a salt thereof. A phosphodiesterase type 5 inhibitor is a drug that blocks the degradative action of phosphodiesterase type 5 on cyclic GMP, e.g., in the smooth muscle cells lining the blood vessels supplying the corpus cavernosum of the penis. Part of the physiological process of erection involves the release of nitric oxide (NO) in vasculature of the corpus cavernosum as a result of sexual stimulation. NO activates the enzyme guanylate cyclase which results in increased levels of cyclic guanosine monophosphate (cGMP), leading to smooth muscle relaxation in blood vessels supplying the corpus cavernosum, resulting in increased blood flow and an erection. Accordingly, PDE5 inhibitors inhibit the degradation of cGMP by phosphodiesterase type 5, increasing bloodflow to the penis during sexual stimulation.

Results from pre-clinical functional and molecular biology studies have shed light on an analogous NO-cGMP biological pathway present in female genital tissue. Deduced by means of immunohistochemistry methods in female tissue sections, the NO-cGMP dependent PDE-5 isoenzyme is expressed in vascular smooth muscle cells of the human corpus cavernous clitoris, the vagina, and the labia minora. These structures play a central role in mediating the female sexual arousal response. Following sexual stimulation, neurotransmitters modulate increased blood flow to the clitoris, vagina, and labia, resulting in increased clitoral intracavernous pressure, tumescence, protrusion of the glans clitoris, and engorgement of the labia minora and vagina. Anatomically, the female clitoris consists of two corpora cavernosa and a glans. Akin to the male penis, each corpus is lined with a fibrous envelope, the tunica albuginea, containing erectile tissue consisting of 40-45% smooth muscle. Lacking a venous plexus, the female clitoris achieves tumescence but not rigidity during sexual arousal.

### Composition

Non-limiting examples of phosphodiesterase type 5 inhibitors include, but are not limited to, avanafil, lodenafil, mirodenafil (pKa of 6.0), sildenafil (or analogs thereof, for example, actetildenafil, hydroxyacetildenafil, or dimethylsildenafil), tadalafil (pKa of 18), vardenafil (pKas of 3.4, 6.7, 8.8, and 14), udenafil (pKa of 10.53), acetildenafil, or thiomethisosildenafil.

The structures of these compounds are respectively shown below:

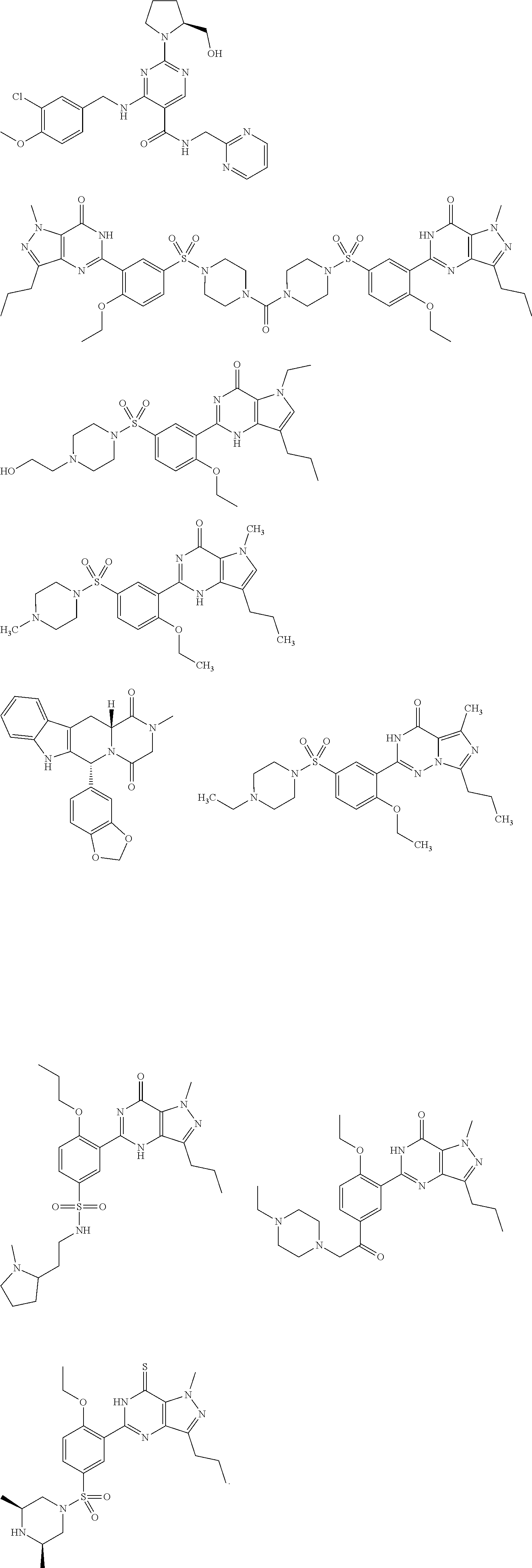

In some embodiments, the phosphodiesterase type 5 inhibitor is sildenafil or salt thereof. In some embodiments, the phosphodiesterase type 5 inhibitor is sildenafil citrate (1-[4-ethoxy-3-(6,7-dihydro-1-methyl-7-oxo-3-propyl-1H-pyrazolo[4,3-d]pyrimidin-5-yl)phenylsulfonyl]-4-methylpiperazine citrate salt). In some embodiments, the phosphodiesterase type 5 inhibitor is avanafil or salt thereof. In some embodiments, the phosphodiesterase type 5 inhibitor is lodenafil or salt thereof. In some embodiments, the phosphodiesterase type 5 inhibitor is mirodenafil or salt thereof. In some embodiments, the phosphodiesterase type 5 inhibitor is hydroxyacetildenafil or salt thereof. In some embodiments, the phosphodiesterase type 5 inhibitor is dimethylsildenafil or salt thereof. In some embodiments, the phosphodiesterase type 5 inhibitor is tadalafil or salt thereof. In some embodiments, the phosphodiesterase type 5 inhibitor is vardenafil or salt thereof. In some embodiments, the phosphodiesterase type 5 inhibitor is udenafil or salt thereof. In some embodiments, the phosphodiesterase type 5 inhibitor is acetildenafil or salt thereof. In some embodiments, the phosphodiesterase type 5 inhibitor is thiomethisosildenafil or salt thereof.

Aspects of the present disclosure provide compositions including a phosphodiesterase type 5 inhibitor for transdermal and/or transmucosal delivery or topical application to a subject. Other compounds such as salts or derivatives of phosphodiesterase type 5 inhibitors (including salts or derivatives of the above compounds) are included in the embodiments; thus, it should be understood that in any embodiment described herein using a phosphodiesterase type 5 inhibitor, this is by way of example only.

Phosphodiesterase type 5 inhibitors or other pharmaceutical agents (e.g., salts or derivatives of phosphodiesterase type 5 inhibitors, etc.) may be present at any suitable concentration. In some embodiments, the pharmaceutical agent is sildenafil citrate. In some embodiments, the pharmaceutical agent may be present at a concentration of about 0.1% to about 10% by weight of the composition. In some embodiments, the pharmaceutical agent may be present at a concentration of about 1% to about 10% by weight of the composition. In some embodiments, the pharmaceutical agent may be present at a concentration of about 1% to about 7% by weight of the composition. In some embodiments, the pharmaceutical agent may be present at a concentration of about 1% to about 5% by weight of the composition. In some embodiments, the pharmaceutical agent may be present at a concentration of about 1% to about 4% by weight of the composition. The concentration may be any value or subrange within the recited ranges, including endpoints.

In some embodiments, the pharmaceutical agent may be present at a concentration of at least about 0.1% by weight of the composition. In some embodiments, the pharmaceutical agent may be present at a concentration of at least about 0.3% by weight of the composition. In some embodiments, the pharmaceutical agent may be present at a concentration of at least about 0.5% by weight of the composition. In some embodiments, the pharmaceutical agent may be present at a concentration of at least about 0.7% by weight of the composition. In some embodiments, the pharmaceutical agent may be present at a concentration of at least about 1% by weight of the composition. In some embodiments, the pharmaceutical agent may be present at a concentration of at least about 2% by weight of the composition. In some embodiments, the pharmaceutical agent may be present at a concentration of at least about 3% by weight of the composition. In some embodiments, the pharmaceutical agent may be present at a concentration of at least about 4% by weight of the composition. In some embodiments, the pharmaceutical agent may be present at a concentration of at least about 5% by weight of the composition. In some embodiments, the pharmaceutical agent may be present at a concentration of at least about 6% by weight of the composition. In some embodiments, the pharmaceutical agent may be present at a concentration of at least about 7% by weight of the composition. In some embodiments, the pharmaceutical agent may be present at a concentration of at least about 7.5% by weight of the composition. In some embodiments, the pharmaceutical agent may be present at a concentration of at least about 8% by weight of the composition. In some embodiments, the pharmaceutical agent may be present at a concentration of at least about 9% by weight of the composition. In some embodiments, the pharmaceutical agent may be present at a concentration of at least about 10% by weight of the composition.

In certain embodiments, the pharmaceutical agent may be present at a concentration of no more than about 1% by weight of the composition. In certain embodiments, the pharmaceutical agent may be present at a concentration of no more than about 2% by weight of the composition. In certain embodiments, the pharmaceutical agent may be present at a concentration of no more than about 3% by weight of the composition. In certain embodiments, the pharmaceutical agent may be present at a concentration of no more than about 4% by weight of the composition. In certain embodiments, the pharmaceutical agent may be present at a concentration of no more than about 5% by weight of the composition.

In certain embodiments, the pharmaceutical agent may be present at a concentration of no more than about 6% by weight of the composition. In certain embodiments, the pharmaceutical agent may be present at a concentration of no more than about 7% by weight of the composition. In certain embodiments, the pharmaceutical agent may be present at a concentration of no more than about 8% by weight of the composition. In certain embodiments, the pharmaceutical agent may be present at a concentration of no more than about 9% by weight of the composition. In certain embodiments, the pharmaceutical agent may be present at a concentration of no more than about 10% by weight of the composition. In certain embodiments, the pharmaceutical agent may be present at a concentration of no more than about 12% by weight of the composition. In certain embodiments, the pharmaceutical agent may be present at a concentration of no more than about 15% by weight of the composition. In certain embodiments, the pharmaceutical agent may be present at a concentration of no more than about 20% by weight of the composition.

In some embodiments, the pharmaceutical agent is a phosphodiesterase type 5 inhibitor. In some embodiments, the phosphodiesterase type 5 inhibitor is sildenafil. In some embodiments, the compositions of the present disclosure include sildenafil citrate and are used in the treatment of FSAD. In certain embodiments, the sildenafil or salt thereof may be present at a concentration of about 1% by weight of the composition. In certain embodiments, the sildenafil or salt thereof may be present at a concentration of about 2% by weight of the composition. In certain embodiments, the sildenafil or salt thereof may be present at a concentration of about 3% by weight of the composition. In certain embodiments, the sildenafil or salt thereof may be present at a concentration of about 3.6% by weight of the composition. In certain embodiments, the sildenafil or salt thereof may be present at a concentration of about 4% by weight of the composition. In some embodiments, the sildenafil or salt thereof is present at a concentration of about 5% by weight of the composition.

In addition, the pharmaceutical agent may be present in native form and/or as one or more salts. For example, if a phosphodiesterase type 5 inhibitor is present, it may be used in its native form, and/or as one or more salts, e.g., the sodium salt, the potassium salt, the magnesium salt, the lysine salt, the arginine salt, the lactate salt, or the citrate salt of a phosphodiesterase type 5 inhibitor (e.g., avanafil, lodenafil, mirodenafil, sildenafil, tadalafil, vardenafil, udenafil, acetildenafil, thiomethisosildenafil, etc.) These may include, for example, citric acid, citric acid monohydrate, sodium citrate, sodium citrate dihydrate, or the like. In some embodiments, sildenafil is present as a sodium salt, potassium salt, magnesium salt, lysine salt, arginine salt, lactate salt, or citrate salt. In some embodiments, sildenafil is present as a citrate salt. As a non-limiting example, in one set of embodiments, the composition may include citrate and/or a citrate salt.

For salt forms of the pharmaceutical agent, “by weight of the composition” includes the entire salt form of the pharmaceutical agent, e.g., the agent itself as well as any counterions such as sodium, potassium, etc. The amount of the pharmaceutical agent may be determined in a composition, for example, using techniques such as HPLC or HPLC/MS that are known to those of ordinary skill in the art.

The composition may also comprise a nitric oxide donor in some embodiments. In some cases, such a nitric oxide donor may be used to increase localized blood flow at the site where the composition is applied, which may enhance delivery of the pharmaceutical agent. The nitric oxide donor may be present at any suitable concentration within the composition. In some cases, the nitric oxide donor is present at a concentration of about 1% to about 20% by weight of the composition. In some cases, the nitric oxide donor is present at a concentration of about 1% to about 10% by weight of the composition. In some cases, the nitric oxide donor is present at a concentration of about 5% to about 10% by weight of the composition. In some cases, the nitric oxide donor is present at a concentration of at least about 1% by weight of the composition. In some cases, the nitric oxide donor is present at a concentration of at least about 2% by weight of the composition. In some cases, the nitric oxide donor is present at a concentration of at least about 3% by weight of the composition. In some cases, the nitric oxide donor is present at a concentration of at least about 4% by weight of the composition. In some cases, the nitric oxide donor is present at a concentration of at least about 5% by weight of the composition. In some cases, the nitric oxide donor is present at a concentration of at least about 6% by weight of the composition. In some cases, the nitric oxide donor is present at a concentration of at least about 7% by weight of the composition. In some cases, the nitric oxide donor is present at a concentration of at least about 7.5% by weight of the composition. In some cases, the nitric oxide donor is present at a concentration of at least about 8% by weight of the composition. In some cases, the nitric oxide donor is present at a concentration of at least about 9% by weight of the composition. In some cases, the nitric oxide donor is present at a concentration of at least about 10% by weight of the composition.

The composition may also comprise L-arginine and/or L-arginine hydrochloride. In embodiments, L-arginine and/or L-arginine hydrochloride is a humectant. In embodiments, any humectant may be used. The L-arginine and/or L-arginine hydrochloride may be present at any suitable concentration within the composition. In some cases, the L-arginine and/or L-arginine hydrochloride is present at a concentration of about 1% to about 20% by weight of the composition. In some cases, the L-arginine and/or L-arginine hydrochloride is present at a concentration of about 1% to about 10% by weight of the composition. In some cases, the L-arginine and/or L-arginine hydrochloride is present at a concentration of about 5% to about 10% by weight of the composition. In some cases, the L-arginine and/or L-arginine hydrochloride is present at a concentration of at least about 1% by weight of the composition. In some cases, the L-arginine and/or L-arginine hydrochloride is present at a concentration of at least about 2% by weight of the composition. In some cases, the L-arginine and/or L-arginine hydrochloride is present at a concentration of at least about 3% by weight of the composition. In some cases, the L-arginine and/or L-arginine hydrochloride is present at a concentration of at least about 4% by weight of the composition. In some cases, the L-arginine and/or L-arginine hydrochloride is present at a concentration of at least about 5% by weight of the composition. In some cases, the L-arginine and/or L-arginine hydrochloride is present at a concentration of at least about 6% by weight of the composition. In some cases, the L-arginine and/or L-arginine hydrochloride is present at a concentration of at least about 7% by weight of the composition. In some cases, the L-arginine and/or L-arginine hydrochloride is present at a concentration of at least about 7.5% by weight of the composition. In some cases, the L-arginine and/or L-arginine hydrochloride is present at a concentration of at least about 8% by weight of the composition. In some cases, the L-arginine and/or L-arginine hydrochloride is present at a concentration of at least about 9% by weight of the composition. In some cases, the L-arginine and/or L-arginine hydrochloride is present at a concentration of at least about 10% by weight of the composition. In some embodiments, L-arginine hydrochloride (HCl) is present at a concentration of about 7.5% by weight of the composition.

In some cases, one or more nitric oxide donors (e.g., 2, 3, 4, 5, 6, 7, 8, 9, 10, etc. nitric oxide donors) may be used. In some cases, there may be no more than 3, 5, 7, or 10 nitric oxide donors present within the composition.

A “nitric oxide donor,” as used herein, is a compound that is able to release nitric oxide and/or chemically transfer the nitric oxide moiety to another molecule, directly or indirectly, for example, through a biological process. The nitric oxide donor may release nitric oxide into the skin, and/or tissues such as muscles and/or elements of the circulatory system in close proximity to the surface of the skin. Non-limiting examples of nitric oxide donors include arginine (e.g., L-arginine and/or D-arginine), arginine derivatives (e.g., L-arginine hydrochloride and/or D-arginine hydrochloride), nitroglycerin, polysaccharide-bound nitric oxide-nucleophile adducts, N-nitroso-N-substituted hydroxylamines, 1,3-(nitrooxymethyl)phenyl-2-hydroxybenzoate, etc., and/or any combination thereof.

Other non-limiting examples of nitric oxide donors include, but not limited to, D,L-arginine, D-arginine, or alkyl (e.g., ethyl, methyl, propyl, isopropyl, butyl, isobutyl, tert-butyl, etc.) esters of L-arginine and/or D-arginine (e.g., a methyl ester, an ethyl ester, a propyl ester, a butyl ester, etc.) and/or salts thereof, as well as other derivatives of arginine and other nitric oxide donors. Non-limiting examples of pharmaceutically acceptable salts include but not limited by, hydrochloride, glutamate, butyrate, or glycolate (e.g., resulting in L-arginine glutamate, L-arginine butyrate, L-arginine glycolate, D-arginine hydrochloride, D-arginine glutamate, etc.). Still other examples of nitric oxide donors include L-arginine-based compounds such as, but not limited to, L-homoarginine, N-hydroxy-L-arginine, nitrosylated L arginine, nitrosylated N-hydroxy-L-arginine, nitrosylated N-hydroxy-Larginine, citrulline, ornithine, linsidomine, nipride, glutamine, etc., and salts thereof (e.g., hydrochloride, glutamate, butyrate, glycolate, etc.), and/or any combination thereof.

In some embodiments, the composition may also comprise an ionic salt. In some embodiments, the composition comprising an ionic salt and a pharmaceutical agent (e.g., a phosphodiesterase type 5 inhibitor, etc.) may create an environment such that the pharmaceutical agent is in a chemically and/or energetically unfavorable environment relative to the skin (e.g., the chemical potential and/or the free energy of the pharmaceutical agent within the composition environment is significantly greater than the chemical potential and/or the free energy of the pharmaceutical agent within the skin, thus energetically favoring transport into the skin), especially the stratum corneum.

Embodiments of the disclosure are directed to compositions for topical delivery to the skin or mucosal membrane of a subject comprising L-arginine and/or L-arginine hydrochloride, an ionic salt, and a pharmaceutical agent such as a phosphodiesterase type 5 inhibitor, or a salt or a derivative of a phosphodiesterase type 5 inhibitor.

Examples of ionic salts include, but are not limited to, lithium chloride, sodium chloride, potassium chloride, calcium chloride, magnesium chloride, choline chloride, sodium fluoride, lithium bromide, and combinations thereof. In some embodiments, the ionic salt is present at a concentration of about 1% to about 10% by weight of the composition. In some embodiments, the ionic salt is present at a concentration of about 2% to about 8% by weight of the composition. In some embodiments, the ionic salt is present at a concentration of at least about 1%, at least about 2%, at least about 3%, at least about 4%, at least about 5%, at least about 6%, at least about 7%, at least about 7.5%, at least about 8%, at least about 9%, or at least about 10% by weight of the composition. In some embodiments, the composition comprises ionic salts at an ionic strength of at least about 0.25 M, at least about 1 M, at least about 2 M, at least about 3 M, at least about 5 M, at least about 10 M, at least about 15 M, at least about 20 M, at least about 25 M, or in some cases, between about 0.25 M and about 15 M, between about 5 M and about 15 M, between about 10 M and about 15 M, etc.

In some embodiments, the ionic salt is potassium chloride. In some embodiments, potassium chloride is present at a concentration of about 5% by weight of the composition.

In some embodiments, the composition may include an antioxidant, which may be able to reduce or inhibit the oxidation of other molecules within the composition. Examples of suitable antioxidants include, but are not limited to, glutathione, vitamin C, and vitamin E as well as enzymes such as catalase, superoxide dismutase and various peroxidases. The antioxidant may be present at a concentration of about 0.1% to about 10% by weight of the composition. The antioxidant may be present at a concentration of about 1% to about 5% by weight of the composition. The antioxidant may be present at a concentration of at least about 0.1%, at least about 0.3%, at least about 0.5%, at least about 0.7%, at least about 1%, at least about 2%, at least about 3%, at least about 4%, or at least about 5% by weight of the composition.

As used herein, a “stabilization polymer” refers to a polymer that comprises xanthan gum, a xanthan gum derivative, and/or a xanthan gum equivalent, for example, KELTROL® BT and/or KELTROL® RD, KELZAN® XC, KELZAN® XCD, KELZAN® D, KELZAN® CC, XANTURAL® 180, and/or XANTURAL® 75.

In some embodiments, a composition of the present disclosure may further include a stabilization polymer, propylene glycol, and a polysorbate surfactant. Non-limiting examples of stabilization polymers include xanthan gum, KELTROL® BT and/or KELTROL® RD; an example of a polysorbate surfactant is Polysorbate 20.

In some embodiments, the stabilization polymer may be present at a concentration of about 0.1% to about 20% by weight of the composition. In some embodiments, the stabilization polymer may be present at a concentration of about 0.1% to about 10% by weight of the composition. In some embodiments, the stabilization polymer may be present at a concentration of about 0.1% to about 1% by weight of the composition. In some embodiments, the stabilization polymer may be present at a concentration of at least about 0.1% by weight of the composition. In some embodiments, the stabilization polymer may be present at a concentration of at least about 0.2% by weight of the composition. In some embodiments, the stabilization polymer may be present at a concentration of at least about 0.3% by weight of the composition. In some embodiments, the stabilization polymer may be present at a concentration of at least about 0.4% by weight of the composition. In some embodiments, the stabilization polymer may be present at a concentration of at least about 0.5% by weight of the composition. In some embodiments, the stabilization polymer may be present at a concentration of at least about 0.6% by weight of the composition. In some embodiments, the stabilization polymer may be present at a concentration of at least about 0.7% by weight of the composition. In some embodiments, the stabilization polymer may be present at a concentration of at least about 0.8% by weight of the composition. In some embodiments, the stabilization polymer may be present at a concentration of at least about 0.9% by weight of the composition. In some embodiments, the stabilization polymer may be present at a concentration of at least about 1% by weight of the composition.

In some embodiments, the stabilization polymer may be present at a concentration of no more than about 0.1% by weight of the composition. In some embodiments, the stabilization polymer may be present at a concentration of no more than about 0.2% by weight of the composition. In some embodiments, the stabilization polymer may be present at a concentration of no more than about 0.4% by weight of the composition. In some embodiments, the stabilization polymer may be present at a concentration of no more than about 0.6% by weight of the composition. In some embodiments, the stabilization polymer may be present at a concentration of no more than about 0.8% by weight of the composition. In some embodiments, the stabilization polymer may be present at a concentration of no more than about 1% by weight of the composition. In some embodiments, the stabilization polymer may be present at a concentration of no more than about 2% by weight of the composition. In some embodiments, the stabilization polymer may be present at a concentration of no more than about 3% by weight of the composition. In some embodiments, the stabilization polymer may be present at a concentration of no more than about 4% by weight of the composition. In some embodiments, the stabilization polymer may be present at a concentration of no more than about 5% by weight of the composition. In some embodiments, the stabilization polymer may be present at a concentration of no more than about 7% by weight of the composition. In some embodiments, the stabilization polymer may be present at a concentration of no more than about 10% by weight of the composition. In some embodiments, the stabilization polymer may be present at a concentration of no more than about 12% by weight of the composition. In some embodiments, the stabilization polymer may be present at a concentration of no more than about 15% by weight of the composition. In some embodiments, the stabilization polymer may be present at a concentration of no more than about 20% by weight of the composition.

In some embodiments, the stabilization polymer is xanthan gum. In some embodiments, xanthan gum is present at a concentration of about 0.8% by weight of the composition.

Propylene glycol may be present in compositions of the present disclosure at a concentration of about 1% to about 25% by weight of the composition. Propylene glycol may be present in compositions of the present disclosure at a concentration of about 1% to about 20% by weight of the composition. Propylene glycol may be present in compositions of the present disclosure at a concentration of about 1% to about 15% by weight of the composition. Propylene glycol may be present in compositions of the present disclosure at a concentration of about 1% to about 10% by weight of the composition. Propylene glycol may be present in compositions of the present disclosure at a concentration of at least about 1% by weight of the composition. Propylene glycol may be present in compositions of the present disclosure at a concentration of at least about 2% by weight of the composition. Propylene glycol may be present in compositions of the present disclosure at a concentration of at least about 3% by weight of the composition. Propylene glycol may be present in compositions of the present disclosure at a concentration of at least about 4% by weight of the composition. Propylene glycol may be present in compositions of the present disclosure at a concentration of at least about 5% by weight of the composition. Propylene glycol may be present in compositions of the present disclosure at a concentration of at least about 6% by weight of the composition. Propylene glycol may be present in compositions of the present disclosure at a concentration of at least about 7% by weight of the composition. Propylene glycol may be present in compositions of the present disclosure at a concentration of at least about 8% by weight of the composition. Propylene glycol may be present in compositions of the present disclosure at a concentration of at least about 8.5% by weight of the composition. Propylene glycol may be present in compositions of the present disclosure at a concentration of at least about 9% by weight of the composition. Propylene glycol may be present in compositions of the present disclosure at a concentration of at least about 10% by weight of the composition.

In some embodiments, propylene glycol may be present at a concentration of no more than about 2% by weight of the composition. In some embodiments, propylene glycol may be present at a concentration of no more than about 2% by weight of the composition. In some embodiments, propylene glycol may be present at a concentration of no more than about 4% by weight of the composition. In some embodiments, propylene glycol may be present at a concentration of no more than about 6% by weight of the composition. In some embodiments, propylene glycol may be present at a concentration of no more than about 8% by weight of the composition. In some embodiments, propylene glycol may be present at a concentration of no more than about 10% by weight of the composition. In some embodiments, propylene glycol may be present at a concentration of no more than about 12% by weight of the composition. In some embodiments, propylene glycol may be present at a concentration of no more than about 15% by weight of the composition. In some embodiments, propylene glycol may be present at a concentration of no more than about 20% by weight of the composition. In some embodiments, propylene glycol may be present at a concentration of no more than about 25% by weight of the composition.

In some cases, other glycols can be used in conjunction or instead of propylene glycol, such as butylene glycol. In the specification herein, references to propylene glycol, in other embodiments, should be understood to also include other glycols (e.g., a low molecular weight glycol or a polyglycol) in conjunction or instead of propylene glycol.

In some embodiments, the propylene glycol is present in compositions of the present disclosure at a concentration of about 8.5% by weight of the composition.

A polysorbate surfactant may be present in compositions of the present disclosure at a concentration of about 1% to about 25% by weight of the composition. A polysorbate surfactant may be present in compositions of the present disclosure at a concentration of about 1% to about 20% by weight of the composition. A polysorbate surfactant may be present in compositions of the present disclosure at a concentration of about 1% to about 15% by weight of the composition. A polysorbate surfactant may be present in compositions of the present disclosure at a concentration of about 1% to about 10% by weight of the composition. A polysorbate surfactant may be present in compositions of the present disclosure at a concentration of about 1% to about 5% by weight of the composition. A polysorbate surfactant may be present in compositions of the present disclosure at a concentration of at least about 1% by weight of the composition. A polysorbate surfactant may be present in compositions of the present disclosure at a concentration of at least about 2% by weight of the composition. A polysorbate surfactant may be present in compositions of the present disclosure at a concentration of at least about 3% by weight of the composition. A polysorbate surfactant may be present in compositions of the present disclosure at a concentration of at least about 4% by weight of the composition. A polysorbate surfactant may be present in compositions of the present disclosure at a concentration of at least about 5% by weight of the composition. A polysorbate surfactant may be present in compositions of the present disclosure at a concentration of at least about 6% by weight of the composition. A polysorbate surfactant may be present in compositions of the present disclosure at a concentration of at least about 7% by weight of the composition. A polysorbate surfactant may be present in compositions of the present disclosure at a concentration of at least about 8% by weight of the composition. A polysorbate surfactant may be present in compositions of the present disclosure at a concentration of at least about 9% by weight of the composition. A polysorbate surfactant may be present in compositions of the present disclosure at a concentration of at least about 10% by weight of the composition.

In certain embodiments, the polylsorbate surfactant may be present at a concentration of no more than about 2% by weight of the composition. In certain embodiments, the polylsorbate surfactant may be present at a concentration of no more than about 4% by weight of the composition. In certain embodiments, the polylsorbate surfactant may be present at a concentration of no more than about 6% by weight of the composition. In certain embodiments, the polylsorbate surfactant may be present at a concentration of no more than about 8% by weight of the composition. In certain embodiments, the polylsorbate surfactant may be present at a concentration of no more than about 10% by weight of the composition. In certain embodiments, the polylsorbate surfactant may be present at a concentration of no more than about 12% by weight of the composition. In certain embodiments, the polylsorbate surfactant may be present at a concentration of no more than about 15% by weight of the composition. In certain embodiments, the polylsorbate surfactant may be present at a concentration of no more than about 20% by weight of the composition. In certain embodiments, the polylsorbate surfactant may be present at a concentration of no more than about 25% by weight of the composition.

A “polysorbate surfactant” as used herein, refers to a surfactant comprising a polysorbate. For example, the surfactant may comprise sorbitan monolaurate, sorbitan monopalmitate, sorbitan monostearate, sorbitan monooleate, or another sorbitan salt.

In some embodiments, the polysorbate surfactant is Polysorbate 20. In some embodiments, the Polysorbate 20 is present at a concentration of about 2% by weight of the composition.

In some embodiments, a pharmaceutical agent may be combined with a penetrating agent, i.e., an agent that increases transport of the pharmaceutical agent into the skin, relative to transport in the absence of the penetrating agent. Examples of penetrating agents include, but are not limited to, oleoresin capsicum cationic, anionic, or nonionic surfactants (e.g., sodium dodecyl sulfate, polyoxamers, etc.); fatty acids and alcohols (e.g., ethanol, oleic acid, lauric acid, liposomes, etc.); anticholinergic agents (e.g., benzilonium bromide, oxyphenonium bromide); alkanones (e.g., n-heptane); amides (e.g., urea, N,N-dimethyl-m-toluamide); fatty acid esters (e.g., n-butyrate); organic acids (e.g., citric acid); polyols (e.g., ethylene glycol, glycerol); sulfoxides (e.g., dimethylsulfoxide); terpenes (e.g., cyclohexene); ureas; sugars; and/or carbohydrates.

In some embodiments, the penetrating agent is oleic acid. In some embodiments, the oleic acid is present at a concentration of about 1% by weight of the composition.

As a specific non-limiting example, a cream may have one or more of (w/w): water (40.75%); potassium chloride (5%); L-Arginine HCl (7.5%); glyceryl stearate (7%); cetyl alcohol (7%); squalane (4%); xanthan gum (0.8%); isopropyl myristate (1%); oleic acid (1%); propylene glycol (8.5%); polysorbate 20 (2%); trisodium citrate dihydrate (10%); sodium benzoate (0.2%); gluconolactone (0.25%); and/or sildenafil citrate (5%).

As a specific non-limiting example, a cream may have one or more of (w/w): water (about 35% to about 55%); potassium chloride (about 2.5% to about 15%); L-Arginine HCl (about 2.5% to about 15%); glyceryl stearate (about 4% to about 10%); cetyl alcohol (about 4% to about 10%); squalane (about 1% to about 8%); xanthan gum (about 0.1% to about 5%); isopropyl myristate (about 0.1% to about 5%); oleic acid (about 0.1% to about 5%); propylene glycol (about 1% to about 10%); polysorbate 20 (about 0.2% to about 5%); trisodium citrate dihydrate (about 5% to about 20%); sodium benzoate (about 0.05% to about 1%); gluconolactone (about 0.05% to about 1%); and/or sildenafil citrate (about 1% to about 10%).

### Delivery

In some embodiments, topical delivery of a phosphodiesterase type 5 inhibitor, for example sildenafil, provides a surprisingly rapid effect (e.g., within about 1-5 minutes). In contrast, an oral counterpart requires about 60 minutes or more to produce an effect. Aspects of the disclosure provide methods and compositions for treating or preventing FSAD or symptoms thereof. In some embodiments, a topical composition is provided that can be applied to a genital area of a female subject (e.g., the vulva and/or intravaginally) to treat an FSAD or symptoms thereof within less than 60 minutes of application. In some embodiments, the topical composition is applied to a genital area of a female subject to treat an FSAD or symptoms thereof within less than 45 minutes of application. In some embodiments, the topical composition is applied to a genital area of a female subject to treat an FSAD or symptoms thereof within less than 30 minutes of application. In some embodiments, the topical composition is applied to a genital area of a female subject to treat an FSAD or symptoms thereof within less than 15 minutes of application. In some embodiments, the topical composition is applied to a genital area of a female subject to treat an FSAD or symptoms thereof within less than 10 minutes of application.

In some embodiments, the composition is a cream. In some embodiments, the composition is a lotion. In some embodiments, the composition is a gel or hydrogel.

Without wishing to be bound to any theory, when a composition is applied to the skin or mucosal membrane of a subject, the pharmaceutical agent exits the vehicle into the tissue readily, as the pharmaceutical agent is dispersed by local blood flow and does not build up in concentration in the tissue. Thus, in certain embodiments, pharmaceutical agents may be introduced into the skin or mucosal membrane, for example, a phosphodiesterase type 5 inhibitor and/or a salt or derivative of a phosphodiesterase type 5 inhibitor, such as avanafil, lodenafil, mirodenafil, sildenafil, tadalafil, vardenafil, udenafil, acetildenafil, or thiomethisosildenafil. The composition may be delivered locally and/or systemically; initially, much of the delivery is at first local (i.e., through the skin or mucosal membrane), but in some cases, the pharmaceutical agents may also be distributed systemically, e.g., upon reaching the blood supply.

Non-limiting examples of delivery vehicles which would be carried into tissue includes liposomes or emulsions of collagen, collagen peptides or other components of skin or basement membrane. Non-limiting examples of neutralization of charge include delivery of the pharmaceutical agent in the form or an ester or salt which is electronically neutral.

In some embodiments, the composition may be present as an emulsion. As known by those of ordinary skill in the art, an emulsion typically includes a first phase (e.g., a discontinuous phase) contained within a second fluid phase (e.g., a continuous phase). The pharmaceutical agent (e.g., a phosphodiesterase type 5 inhibitor) may be present in either or both phases. In some embodiments, an emulsion is prepared by mixing a first aqueous preparation (e.g., a water phase) with a second non-aqueous preparation (e.g., an oil or lipid phase). Pharmaceutical agents that are water-soluble may be added to the first aqueous preparation (e.g., prior to mixing with the second non-aqueous preparation). Pharmaceutical agents that are water insoluble (or relatively water insoluble) may be added to the second non-aqueous preparation (e.g., prior to mixing with the first aqueous preparation). Pharmaceutical agents that are partially water soluble may be added to one phase, or may be split between the two phases prior to mixing. The split between the two phases will depend on the amount of pharmaceutical agent that is being added, the composition (e.g., the nature and the amount of other chemicals or agents) of the first and second preparations, the pH, the temperature, other physical or chemical factors, and/or a combination thereof.

Emulsions of the disclosure may be packaged using any suitable format (e.g., in a tube, a pump-actuated container, or any other suitable form). In some embodiments, an emulsion may be added to a surface of a patch or bandage. The emulsion may also be applied to the skin or mucosal membrane of a subject as a cream, gel, liquid, lotion, spray, aerosol, transdermal patch, or transmucosal patch. In some embodiments, the compositions described herein are a cream.

In another aspect, the present disclosure is directed to a kit including one or more of the compositions discussed herein. A “kit,” as used herein, refers to a package or an assembly including one or more of the compositions of the disclosure, and/or other compositions associated with the disclosure, for example, as described herein. Each of the compositions of the kit may be provided in liquid form (e.g., in solution), or in solid form (e.g., a dried powder). In certain cases, some of the compositions may be constitutable or otherwise processable (e.g., to an active form), for example, by the addition of a suitable solvent or other species, which may or may not be provided with the kit. Examples of other compositions or components associated with the disclosure include, but are not limited to, solvents, surfactants, diluents, salts, buffers, emulsifiers, chelating agents, fillers, antioxidants, binding agents, bulking agents, preservatives, drying agents, antimicrobials, needles, syringes, packaging materials, tubes, bottles, flasks, beakers, dishes, frits, filters, rings, clamps, wraps, patches, containers, and the like, for example, for using, administering, modifying, assembling, storing, packaging, preparing, mixing, diluting, and/or preserving the compositions components for a particular use, for example, to a sample and/or a subject.

A kit of the disclosure may, in some cases, include instructions in any form that are provided in connection with the compositions of the disclosure in such a manner that one of ordinary skill in the art would recognize that the instructions are to be associated with the compositions of the disclosure. For instance, the instructions may include instructions for the use, modification, mixing, diluting, preserving, administering, assembly, storage, packaging, and/or preparation of the compositions and/or other compositions associated with the kit. In some cases, the instructions may also include instructions for the delivery and/or administration of the compositions, for example, for a particular use, e.g., to a sample and/or a subject.

### Methods of Use

The present disclosure provides a method, comprising: applying, to the genital area of a subject, a composition comprising an active ingredient for treating Female Sexual Dysfunction (FSD). In some embodiments, the active ingredient is sildenafil. In some embodiments, the subject has FSD. In some embodiments, the subject is at risk of FSD. In some embodiments, the subject has one or more symptoms of FSD.

The present disclosure provides a method, comprising: applying, to the genital area of a subject, a composition comprising an active ingredient for treating Female Sexual Arousal Disorder (FSAD). In some embodiments, the active ingredient is sildenafil. In some embodiments, the subject has FSAD. In some embodiments, the subject is at risk of FSAD. In some embodiments, the subject has one or more symptoms of FSAD.

The present disclosure provides a method, comprising: applying, to the genital area of a subject, a composition comprising an active ingredient for treating Female Sexual Interest/Arousal Disorder (FSIAD). In some embodiments, the active ingredient is sildenafil. In some embodiments, the subject has FSIAD. In some embodiments, the subject is at risk of FSIAD. In some embodiments, the subject has one or more symptoms of FSIAD.

In some embodiments, the composition includes propylene glycol, a stabilization polymer, a polysorbate surfactant, L-arginine or L-arginine salt, and an ionic salt. In some embodiments, the composition is a cream. In some embodiments, the stabilization polymer is xanthan gum. In some embodiments, the polysorbate surfactant is polysorbate 20. In some embodiments, the composition further includes a nitric oxide donor. In some embodiments, the ionic salt is potassium chloride. In some embodiments, the active ingredient is present at about 1-10 wt %. In some embodiments, the composition is administered to a clitoris, vulva, vagina and/or intravaginally. In some embodiments, the composition is applied to a clitoris, vestibule, vulva, and/or vagina of the subject. In some embodiments, the composition is applied to a clitoris, glans, and/or frenulum of a genital area of the subject. In some embodiments, the composition is applied to a clitoris, glans, frenulum, and/or vestibule of a genital area of the subject. In some embodiments, the composition is applied to a clitoris, glans, frenulum, vestibule, and/or labia minora of a genital area of the subject. In some embodiments, the composition is not applied to a labia majora of a genital area of the subject. In some embodiments, the composition is applied intravaginally to the anterior distal vagina of the subject. In some embodiments, the composition is applied as a single dose. In some embodiments, the composition is applied as at least one dose.

Accordingly, as discussed above, a variety of compositions are covered in various embodiments, including any suitable combinations of any of the above-described components, such as xanthan gum and/or another stabilization polymer, propylene glycol, polysorbate 20 and/or another polysorbate surfactant, L-arginine and/or nitric oxide donor, potassium chloride and/or another ionic salt, an active ingredient, and water. The active ingredient may include any of those described herein, for example sildenafil, as well as salts thereof.

Furthermore, as mentioned, certain aspects as described herein are generally directed to compositions and methods for applying such compositions for the treatment or prevention of indications such as any of those described herein, e.g., to the vulva and/or vagina of a subject, such as a human. In embodiments, the subject is female.

In another set of embodiments, a composition such is described herein may be used to treat a subject having or at risk of FSAD. In embodiments, the composition is administered to a subject at least one time.

In some embodiments, a method for treating FSD or symptom thereof comprises selecting a female subject exhibiting one or more symptom of FSD, and administering to a genital of the subject a composition comprising a phosphodiesterase type 5 inhibitor. In embodiments, the composition includes a salt thereof, a stabilization polymer, propylene glycol, a polysorbate surfactant, L-arginine or L-arginine salt or other humectant, and an ionic salt.

In some embodiments, a method for treating Female Sexual Arousal Disorder (FSAD) or symptom thereof comprises selecting a female subject exhibiting one or more symptom of FSAD, and administering to a genital of the subject a composition comprising a phosphodiesterase type 5 inhibitor. In embodiments, the composition includes a salt thereof, a stabilization polymer, propylene glycol, a polysorbate surfactant, L-arginine or L-arginine salt, and an ionic salt.

In some embodiments, symptoms of FSAD include, but are not limited to, decreased genital sensitivity, lack of genital responsiveness during sexual activity, diminished genital arousal, distress regarding sexual arousal difficulties, and any combination thereof. In some embodiments, compositions of the present disclosure are administered the genital area of a subject including, but not limited to the clitoris, vestibule, vulva, and intravaginally. In embodiments, the one or more symptoms includes reduced response to physical sexual stimulation. In embodiments, the female subject's primary complaint is reduced response to physical sexual stimulation.

In some embodiments, treating FSAD includes increasing satisfactory sexual events, improving arousal sensation, increasing arousal lubrication, increasing orgasm, decreasing concern with sexual arousal, and/or improving satisfaction with sexual activity. In some embodiments, treating FSAD includes improving arousal sensation. In some embodiments, treating FSAD includes increasing arousal lubrication. In some embodiments, treating FSAD includes increasing orgasm. In some embodiments, treating FSAD includes decreasing concern with sexual arousal. In some embodiments, treating FSAD includes improving satisfaction with sexual activity.

In some embodiments, treating FSAD includes increasing satisfactory sexual events. In embodiments, increasing satisfactory sexual events includes, but is not limited to, increasing orgasm or orgasm frequency, feeling close to or connection with a sexual partner, experiencing pleasurable sensations, increased physical sensation, feelings of arousal, feelings of passion, and/or having an enjoyable sexual experience.

In some embodiments, treating FSAD includes increasing sensation at the genital area. In some embodiments, sensation at the genital are includes one or more of: tingling, pleasure, warmth, pulsating, throbbing, engorgement, or fullness at the genital area.

In some aspects, described herein is a method for increasing satisfactory sexual events in a female subject during sexual activity, comprising applying to a genital area of a female subject a topical composition comprising a phosphodiesterase type 5 inhibitor and/or a salt thereof.

In some aspects, described herein is a method for improving arousal sensation in a female subject during sexual activity, comprising applying to a genital area of a female subject a topical composition comprising a phosphodiesterase type 5 inhibitor and/or a salt thereof.

In some aspects, described herein is a method for increasing arousal lubrication in a female subject during sexual activity, comprising applying to a genital area of a female subject a topical composition comprising a phosphodiesterase type 5 inhibitor and/or a salt thereof.

In some aspects, described herein is a method for increasing orgasm in a female subject during sexual activity, comprising applying to a genital area of a female subject a topical composition comprising a phosphodiesterase type 5 inhibitor and/or a salt thereof.

In some aspects, described herein is a method for decreasing concern with sexual arousal or improving satisfaction with sexual activity in a female subject, comprising applying to a genital area of a female subject a topical composition comprising a phosphodiesterase type 5 inhibitor and/or a salt thereof.

In some aspects, described herein is a method for decreasing pain or discomfort during and/or after sexual activity in a female subject, comprising applying to a genital area of a female subject a topical composition comprising a phosphodiesterase type 5 inhibitor and/or a salt thereof.

In embodiments, a method for treating FSIAD or symptom thereof comprises selecting a female subject exhibiting one or more symptom of FSIAD, and administering to a genital of the subject a composition comprising a phosphodiesterase type 5 inhibitor. In embodiments, the composition includes a salt thereof, a stabilization polymer, propylene glycol, a polysorbate surfactant, a L-arginine or L-arginine salt, and an ionic salt.

In embodiments, treating FSIAD comprises increasing sexual desire. In embodiments, treating FSIAD comprises increasing response to physical or mental sexual stimulation. In embodiments, the symptom of FSIAD comprises absent or reduced interest in sexual activity. In embodiments, the symptom of FSIAD comprises absent or reduced erotic thoughts or fantasies. In embodiments, the symptom of FSIAD comprises reduced sexual desire. In embodiments, the symptom of FSIAD comprises reduced response to physical sexual stimulation. In embodiments, the symptom of FSIAD comprises reduced response to mental sexual stimulation.

In embodiments, improvement in one or more criteria (e.g. symptoms) described herein is measured based on Female Sexual Distress Scale-Desire, Arousal, Orgasm (FSDS-DAO) score. In embodiments, improvement in one or more criteria (e.g. symptoms) described herein is measured based on Female Sexual Distress Scale-Desire, Arousal, Orgasm (FSDS-DAO), Item 14 score (feeling concerned by difficulties with sexual arousal). In embodiments, improvement means FSDS-DAO score increased by at least one point. In embodiments, improvement means FSDS-DAO, Item 14 score increased by at least one point.

In embodiments, improvement in one or more criteria (e.g. symptoms) described herein is measured based on Sexual Function Questionnaire (SFQ28) score. In embodiments, improvement in one or more criteria (e.g. symptoms) described herein is measured based on SFQ28 domain item 1 (warmth frequency) score. In embodiments, improvement in one or more criteria (e.g. symptoms) described herein is measured based on SFQ28 domain item 2 (warmth degree) score. In embodiments, improvement in one or more criteria (e.g. symptoms) described herein is measured based on SFQ28 domain item 3 (pulsating/tingling frequency) score. In embodiments, improvement in one or more criteria (e.g. symptoms) described herein is measured based on SFQ28 domain item 4 (pulsating/tingling degree) score. In embodiments, improvement means SFQ28 score or domain item score increased by at least one point. In embodiments, improvement means SFQ28 score or domain item score increased by at least two points.

In embodiments, improvement in one or more criteria (e.g. symptoms) described herein is measured based on Arousal Diary (AD) score. In embodiments, improvement in one or more criteria (e.g. symptoms) described herein is measured based on AD item 1 (pleasurable sensation) score. In embodiments, improvement in one or more criteria (e.g. symptoms) described herein is measured based on AD item 2 (warmth) score. In embodiments, improvement in one or more criteria (e.g. symptoms) described herein is measured based on AD item 3 (pulsating/throbbing sensation) score. In embodiments, improvement in one or more criteria (e.g. symptoms) described herein is measured based on AD item 4 (tingling sensation) score. In embodiments, improvement in one or more criteria (e.g. symptoms) described herein is measured based on AD item 5 (engorgement/fullness) score. In embodiments, improvement means AD score or item score increased by at least one point. In embodiments, improvement means AD score or item score increased by at least two points.

In embodiments, improvement in one or more criteria (e.g. symptoms) described herein is measured based on Patient Global Impression of Change (PGI-C). In embodiments, improvement in one or more criteria (e.g. symptoms) described herein is measured based on description by the subject that symptoms were “a little better.” In embodiments, improvement in one or more criteria (e.g. symptoms) described herein is measured based on description by the subject that symptoms were “much better.” In embodiments, improvement in one or more criteria (e.g. symptoms) described herein is measured based on description by the subject that symptoms were “very much better.”

In embodiments, improvement in one or more criteria (e.g. symptoms) described herein is measured based on Patient Global Impression of Severity (PGI-S)

In embodiments, improvement in one or more criteria (e.g. symptoms) described herein is measured based on Patient Benefit Evaluation (PBE) (meaningful benefit from study medication).

In some embodiments, administering to a genital area of the subject a composition comprising a phosphodiesterase type 5 inhibitor and/or a salt thereof results in increased sexual arousal.

In some embodiments, administering to a genital area of the subject a composition comprising a phosphodiesterase type 5 inhibitor and/or a salt thereof results in increased sexual excitement.

In some embodiments, administering to a genital area of the subject a composition comprising a phosphodiesterase type 5 inhibitor and/or a salt thereof results in a satisfactory sexual event.

In some embodiments, administering to a genital area of the subject a composition comprising a phosphodiesterase type 5 inhibitor and/or a salt thereof results in increased genital arousal sensations.

In some embodiments, administering to a genital area of the subject a composition comprising a phosphodiesterase type 5 inhibitor and/or a salt thereof results in increased lubrication of the genital area during sexual activity.

In some embodiments, administering to a genital area of the subject a composition comprising a phosphodiesterase type 5 inhibitor and/or a salt thereof results in increased cognitive sexual arousal.

In some embodiments, administering to a genital area of the subject a composition comprising a phosphodiesterase type 5 inhibitor and/or a salt thereof results in increased sexual desire.

In some embodiments, administering to a genital area of the subject a composition comprising a phosphodiesterase type 5 inhibitor and/or a salt thereof results in increased orgasm during sexual activity.

In some embodiments, administering to a genital area of the subject a composition comprising a phosphodiesterase type 5 inhibitor and/or a salt thereof results in decreased difficulty or inability to orgasm during sexual activity.

In some embodiments, administering to a genital area of the subject a composition comprising a phosphodiesterase type 5 inhibitor and/or a salt thereof results in increased feeling or sensation of the genital area during sexual activity.

In some embodiments, administering to a genital area of the subject a composition comprising a phosphodiesterase type 5 inhibitor and/or a salt thereof results in increased tingling of the genital area during sexual activity.

In some embodiments, administering to a genital area of the subject a composition comprising a phosphodiesterase type 5 inhibitor and/or a salt thereof results in increased engorgement of the genital area during sexual activity.

In some embodiments, administering to a genital area of the subject a composition comprising a phosphodiesterase type 5 inhibitor and/or a salt thereof results in increased warmth, throbbing, or pulsating of the genital area during sexual activity.

In some embodiments, administering to a genital area of the subject a composition comprising a phosphodiesterase type 5 inhibitor and/or a salt thereof results in decreased pain and/or discomfort during sexual activity.

In some embodiments, administering to a genital area of the subject a composition comprising a phosphodiesterase type 5 inhibitor and/or a salt thereof results in decreased pain and/or discomfort after sexual activity.

In some embodiments, administering to a genital area of the subject a composition comprising a phosphodiesterase type 5 inhibitor and/or a salt thereof results in increased sexual excitement.

In some embodiments, one or more symptoms of Female Sexual Arousal Disorder (FSAD) includes, but is not limited to, reduced response to physical sexual stimulation.

In some embodiments, the female subject's primary complaint is reduced response to physical sexual stimulation.

In some embodiments, a subject exhibits one or more of the following after administration of a composition comprising a phosphodiesterase type 5 inhibitor and/or a salt
a. improvement in Sexual Function Questionnaire (SFQ28) arousal cognitive domain score of at least 1;
b. improvement in SFQ28 arousal lubrication domain score of at least 1;
c. improvement in SFQ28 arousal sensation domain score of at least 1.5;
d. improvement in SFQ28 desire domain score of at least 2;
e. improvement in SFQ28 orgasm domain score of at least 1.20;
f. improvement in SFQ28 pain domain score of at least 1;
g. improvement in Female Sexual Distress Scale-Desire, Arousal, Orgasm (FSDS-DAO) score of at least-7;
h. improvement in an arousal sensation score of at least 1.45;
i. improvement in genital arousal score of at least 1.13;
j. improvement in genital arousal concerns score of at least 1.49;
k. improvement in Arousal Diary Item 12-Proportion of SSEs of at least 0.25; and
l. improvement in Arousal Diary Item 12-Number of SSEs of at least 1.41.

In some embodiments, a subject comprises a primary diagnosis of FSAD as defined by the Diagnostic and Statistical Manual of Mental Disorders Text Revision Fourth Edition (DSM-IV-TR).

In some embodiments, a topical composition comprising a phosphodiesterase type 5 inhibitor and/or a salt thereof does not result in one or more of orthostatic hypotension, headache, flushing, dyspepsia, light headedness, abnormal vision, nasal congestion, back pain, myalgia, nausea, dizziness, and rash.

In some embodiments, a subject's primary complaint is FSAD or one of more symptoms thereof.

In some embodiments, a subject's primary complaint is FSAD or one of more symptoms thereof and the subject exhibits secondary HSDD symptoms. In some embodiments, a subject has FSAD as their sole diagnosis. In some embodiments, a subject has primary FSAD and secondary HSDD. In some embodiments, a premenopausal subject has FSAD as their sole diagnosis. In some embodiments, a premenopausal subject has primary FSAD and secondary HSDD.

The present disclosure generally relates to a method for treating Female Sexual Arousal Disorder (FSAD) or symptom thereof, comprising:
a. selecting a female subject having a primary complaint of FSAD, and
b. applying to a genital area of the subject a topical composition comprising a phosphodiesterase type 5 inhibitor and/or a salt thereof.

In some embodiments, the subject exhibits secondary HSDD symptoms. In some embodiments, a subject's primary complaint is FSAD and the subject does not exhibit Female Orgasmic Disorder (FOD) symptoms (or has not been diagnosed with FOD). In some embodiments, the subject does not exhibit FOD symptoms (or has not been diagnosed with FOD). In some embodiments, the method includes selecting a female subject who does not have a complaint of FOD.

The present disclosure generally relates to a method of determining efficacy of a FSAD treatment, comprising:
a) obtaining a baseline score for one or more of: a Sexual Function Questionnaire (SFQ28) arousal cognitive domain score, a SFQ28 arousal lubrication domain score, a SFQ28 arousal sensation domain score, a SFQ28 desire domain score, a SFQ28 orgasm domain score, a SFQ28 pain domain score, a Female Sexual Distress Scale-Desire, Arousal, Orgasm (FSDS-DAO) score, an arousal sensation score, a genital arousal score, genital arousal concerns, Arousal Diary Item 12-Proportion of SSEs, and Arousal Diary Item 12-Number of SSEs;
b) administering the FSAD treatment to a female subject; and
c) determining improvement in one or more of the scores in step a.

In some embodiments, one or more of the following indicates the FSAD treatment is effective:
a. improvement in Sexual Function Questionnaire (SFQ28) arousal cognitive domain score of at least 1;
b. improvement in SFQ28 arousal lubrication domain score of at least 1;
c. improvement in SFQ28 arousal sensation domain score of at least 1.5;
d. improvement in SFQ28 desire domain score of at least 2;
e. improvement in SFQ28 orgasm domain score of at least 1.20;
f. improvement in SFQ28 pain domain score of at least 1;
g improvement in Female Sexual Distress Scale-Desire, Arousal, Orgasm (FSDS-DAO) score of at least-7;
h. improvement in arousal sensation of at least 1.45;
i. improvement in genital arousal of at least 1.13;
j. improvement in genital arousal concerns of at least 1.49;
k. improvement in Arousal Diary Item 12-Proportion of SSEs of at least 0.25; and
l. improvement in Arousal Diary Item 12-Number of SSEs of at least 1.41.

In some embodiments, the methods disclosed herein comprise applying one or more additional doses of the topical FSAD treatment composition to the subject.

In embodiments, improvement is measured compared to sexual activity without treatment. In embodiments, improvement is measured compared to sexual activity prior to treatment. In embodiments, improvement is measured after a single treatment (e.g., single application). In embodiments, improvement is measured after multiple treatments (e.g., multiple applications). In embodiments, improvement is measured after a multiple treatments over a period of time. In embodiments, the period of time is at least one week. In embodiments, the period of time is between one week and twenty weeks. In embodiments, the period of time is between one week and eight weeks. In embodiments, improvement is measured after between one and 100 treatments. In embodiments, improvement is measured after between one and 50 treatments. In embodiments, improvement is measured after between one and 20 treatments. In embodiments, improvement is measured after between one and 10 treatments.

Compositions disclosed herein may comprise a sildenafil citrate cream. In some embodiments, the sildenafil citrate cream contains about 1% to about 5% sildenafil citrate by weight. In some embodiments, the sildenafil citrate cream contains about 3.6% sildenafil citrate by weight.

In some embodiments, about 2 grams to about 5 grams of the composition, e.g. sildenafil citrate cream, is administered to the subject. In some embodiments, the single unit dosage of the composition, e.g. sildenafil citrate cream, is about 2 grams to about 5 grams. In some embodiments, about 2 grams of the composition, e.g. sildenafil citrate cream, is administered to the subject. In some embodiments, the single unit dosage of the composition, e.g. sildenafil citrate cream, is about 2 grams.

In some embodiments, about 0.5 grams of the composition, e.g. sildenafil citrate cream, is administered to the subject. In some embodiments, about 1 grams of the composition, e.g. sildenafil citrate cream, is administered to the subject. In some embodiments, about 1.5 grams of the composition, e.g. sildenafil citrate cream, is administered to the subject. In some embodiments, about 2 grams of the composition, e.g. sildenafil citrate cream, is administered to the subject. In some embodiments, about 2.5 grams of the composition, e.g. sildenafil citrate cream, is administered to the subject. In some embodiments, about 3 grams of the composition, e.g. sildenafil citrate cream, is administered to the subject. In some embodiments, about 3.5 grams of the composition, e.g. sildenafil citrate cream, is administered to the subject. In some embodiments, about 4 grams of the composition, e.g. sildenafil citrate cream, is administered to the subject. In some embodiments, about 4.5 grams of the composition, e.g. sildenafil citrate cream, is administered to the subject. In some embodiments, about 5 grams of the composition, e.g. sildenafil citrate cream, is administered to the subject. In some embodiments, about 0.5 grams to about 3 grams of the composition, e.g. sildenafil citrate cream, is administered to the subject. In some embodiments, about 1 gram to about 4 grams of the composition, e.g. sildenafil citrate cream, is administered to the subject. In some embodiments, about 2 grams to about 5 grams of the composition, e.g. sildenafil citrate cream, is administered to the subject. In some embodiments, about 3 grams to about 6 grams of the composition, e.g. sildenafil citrate cream, is administered to the subject. In some embodiments, about 4 grams to about 7 grams of the composition, e.g. sildenafil citrate cream, is administered to the subject.

In some embodiments, the single unit dosage of the composition, e.g. sildenafil citrate cream, is about 1 gram. In some embodiments, the single unit dosage of the composition, e.g. sildenafil citrate cream, is about 1.5 grams. In some embodiments, the single unit dosage of the composition, e.g. sildenafil citrate cream, is about 2 grams. In some embodiments, the single unit dosage of the composition, e.g. sildenafil citrate cream, is about 2.5 grams. In some embodiments, the single unit dosage of the composition, e.g. sildenafil citrate cream, is about 3 grams. In some embodiments, the single unit dosage of the composition, e.g. sildenafil citrate cream, is about 3.5 grams. In some embodiments, the single unit dosage of the composition, e.g. sildenafil citrate cream, is about 4 grams. In some embodiments, the single unit dosage of the composition, e.g. sildenafil citrate cream, is about 4.5 grams. In some embodiments, the single unit dosage of the composition, e.g. sildenafil citrate cream, is about 5 grams.

In some embodiments, one half of the single unit dosage of the composition is applied to the external vaginal tissue. In some embodiments, one half of the single unit dosage of the composition is applied intravaginally.

In some embodiments, at least about 10% of the composition is applied to the external vaginal tissue. In some embodiments, at least about 20% of the composition is applied to the external vaginal tissue. In some embodiments, at least about 30% of the composition is applied to the external vaginal tissue. In some embodiments, at least about 40% of the composition is applied to the external vaginal tissue. In some embodiments, at least about 50% of the composition is applied to the external vaginal tissue. In some embodiments, at least about 60% of the composition is applied to the external vaginal tissue. In some embodiments, at least about 70% of the composition is applied to the external vaginal tissue. In some embodiments, at least about 80% of the composition is applied to the external vaginal tissue. In some embodiments, at least about 90% of the composition is applied to the external vaginal tissue. In some embodiments, at least about 100% of the composition is applied to the external vaginal tissue.

External vaginal tissue (e.g., vulva) includes, but is not limited to, anterior labial commissure, prepuce of the clitoris, glans, frenulum, the vestibule of the vagina, the labia majora, and the labia minora. In some embodiments, about 50% of the composition is applied to the external vaginal tissue. In some embodiments, about 50% of the composition is applied to the clitoris, vestibule, vulva, and/or vagina of the subject. In some embodiments, about 50% of the composition is applied to the clitoris, glans, and/or frenulum of a genital area of the subject. In some embodiments, about 50% of the composition is applied to the clitoris, glans, frenulum, and/or vestibule of a genital area of the subject. In some embodiments, about 50% of the composition is applied to the clitoris, glans, frenulum, vestibule, and/or labia minora of a genital area of the subject.

In some embodiments, at least about 10% of the composition is applied intravaginally. In some embodiments, at least about 20% of the composition is applied intravaginally. In some embodiments, at least about 30% of the composition is applied intravaginally. In some embodiments, at least about 40% of the composition is applied intravaginally. In some embodiments, at least about 50% of the composition is applied intravaginally. In some embodiments, at least about 60% of the composition is applied intravaginally. In some embodiments, at least about 70% of the composition is applied intravaginally. In some embodiments, at least about 80% of the composition is applied intravaginally. In some embodiments, at least about 90% of the composition is applied intravaginally. In some embodiments, at least about 100% of the composition is applied intravaginally.

In some embodiments about 50% of the composition is applied intravaginally. In some embodiments about 50% of the composition is applied to the external vaginal tissue and about 50% of the composition is applied intravaginally.

In some embodiments about 10% of the composition is applied to the external vaginal tissue and about 90% of the composition is applied intravaginally. In some embodiments about 20% of the composition is applied to the external vaginal tissue and about 80% of the composition is applied intravaginally. In some embodiments about 30% of the composition is applied to the external vaginal tissue and about 70% of the composition is applied intravaginally. In some embodiments about 40% of the composition is applied to the external vaginal tissue and about 60% of the composition is applied intravaginally. In some embodiments about 60% of the composition is applied to the external vaginal tissue and about 40% of the composition is applied intravaginally. In some embodiments about 70% of the composition is applied to the external vaginal tissue and about 30% of the composition is applied intravaginally. In some embodiments about 80% of the composition is applied to the external vaginal tissue and about 20% of the composition is applied intravaginally. In some embodiments about 90% of the composition is applied to the external vaginal tissue and about 10% of the composition is applied intravaginally. In some embodiments about 100% of the composition is applied to the external vaginal tissue and about 0% of the composition is applied intravaginally. In some embodiments about 0% of the composition is applied to the external vaginal tissue and about 100% of the composition is applied intravaginally.

In some embodiments, the composition is not applied to the labia majora or dermis bearing pubic hair of the subject.

In some embodiments, the composition is applied intravaginally to the anterior distal vagina at a depth of between about 0 cm and about 3 cm in the vagina. In some embodiments, the composition is applied intravaginally to the anterior distal vagina at a depth of between about 0 cm and about 1 cm. In some embodiments, the composition is applied intravaginally to the anterior distal vagina at a depth of between about 0 cm and about 1.5 cm. In some embodiments, the composition is applied intravaginally to the anterior distal vagina at a depth of between about 0 cm and about 2 cm. In some embodiments, the composition is applied intravaginally to the anterior distal vagina at a depth of between about 0 cm and about 2.5 cm. In some embodiments, the composition is applied intravaginally to the anterior distal vagina at a depth of between about 0 cm and about 3 cm. In some embodiments, the composition is applied intravaginally to the anterior distal vagina at a depth of between about 0 cm and about 4 cm. In some embodiments, the composition is applied intravaginally to the anterior distal vagina at a depth of between about 0 cm and about 5 cm. In some embodiments, the composition is applied intravaginally to the anterior distal vagina at a depth of between about 0 cm and about 6 cm. In some embodiments, the composition is applied intravaginally to the anterior distal vagina at a depth of between about 0 cm and about 7 cm. In some embodiments, the composition is applied intravaginally to the anterior distal vagina at a depth of between about 0 cm and about 8 cm.

In some embodiments, the composition is administered between about 1 minute to about 2 hours prior to a sexual activity. In some embodiments, the composition is administered between about 10 minutes to about 2 hours prior to a sexual activity. In some embodiments, the composition is administered between about 10 minutes to about 1 hour prior to a sexual activity. In some embodiments, the composition is administered between about 10 minutes to about 50 minutes prior to a sexual activity. In some embodiments, the composition is administered between about 10 minutes to about 40 minutes prior to a sexual activity. In some embodiments, the composition is administered between about 10 minutes to about 30 minutes prior to a sexual activity. In some embodiments, the composition is administered between about 10 minutes to about 20 minutes prior to a sexual activity. In some embodiments, the composition is administered about 1 minute prior to a sexual activity. In some embodiments, the composition is administered about 2 minutes prior to a sexual activity. In some embodiments, the composition is administered about 5 minutes prior to a sexual activity. In some embodiments, the composition is administered about 10 minutes prior to a sexual activity. In some embodiments, the composition is administered about 15 minutes prior to a sexual activity. In some embodiments, the composition is administered about 20 minutes prior to a sexual activity. In some embodiments, the composition is administered about 25 minutes prior to a sexual activity. In some embodiments, the composition is administered about 30 minutes prior to a sexual activity. In some embodiments, the composition is administered about 35 minutes prior to a sexual activity. In some embodiments, the composition is administered about 40 minutes prior to a sexual activity. In some embodiments, the composition is administered about 45 minutes prior to a sexual activity. In some embodiments, the composition is administered about 50 minutes prior to a sexual activity. In some embodiments, the composition is administered about 55 minutes prior to a sexual activity. In some embodiments, the composition is administered about 60 minutes prior to a sexual activity.

In some embodiments, the composition is administered at most about nine doses in about 4 weeks. In some embodiments, the composition is administered at most about 20 doses in about 4 weeks. In some embodiments, the composition is administered at most about 19 doses in about 4 weeks. In some embodiments, the composition is administered at most about 18 doses in about 4 weeks. In some embodiments, the composition is administered at most about 15 doses in about 4 weeks. In some embodiments, the composition is administered at most about 12 doses in about 4 weeks. In some embodiments, the composition is administered at most about 10 doses in about 4 weeks. In some embodiments, the composition is administered at most about 8 doses in about 4 weeks. In some embodiments, the composition is administered at most about 7 doses in about 4 weeks. In some embodiments, the composition is administered at most about 6 doses in about 4 weeks. In some embodiments, the composition is administered at most about 5 doses in about 4 weeks. In some embodiments, the composition is administered at most about 4 doses in about 4 weeks. In some embodiments, the composition is administered at most about 3 doses in about 4 weeks. In some embodiments, the composition is administered at most about 2 doses in about 4 weeks. In some embodiments, the composition is administered at least about 24 hours before a second dose of the composition.

In some embodiments, a first dose of the composition is administered at least about 5, at least about 10 before a second dose of the composition. In some embodiments, a first dose of the composition is administered at least about 12 before a second dose of the composition. In some embodiments, a first dose of the composition is administered at least about 15 before a second dose of the composition. In some embodiments, a first dose of the composition is administered at least about 20 before a second dose of the composition. In some embodiments, a first dose of the composition is administered at least about 24 before a second dose of the composition. In some embodiments, a first dose of the composition is administered at least about 36 before a second dose of the composition. In some embodiments, a first dose of the composition is administered at least about 40 before a second dose of the composition. In some embodiments, a first dose of the composition is administered at least about 48 before a second dose of the composition. In some embodiments, a first dose of the composition is administered at least about 60 hours before a second dose of the composition.

In embodiments, the phosphodiesterase type 5 inhibitor and/or salt thereof is administered at least twice over a period of time. In embodiments, the phosphodiesterase type 5 inhibitor and/or salt thereof is administered at least three times over a period of time. In embodiments, the phosphodiesterase type 5 inhibitor and/or salt thereof is administered at least four times over a period of time. In embodiments, the phosphodiesterase type 5 inhibitor and/or salt thereof is administered at least five times over a period of time. In embodiments, the phosphodiesterase type 5 inhibitor and/or salt thereof is administered at least six times over a period of time. In embodiments, the phosphodiesterase type 5 inhibitor and/or salt thereof is administered at least seven times over a period of time. In embodiments, the phosphodiesterase type 5 inhibitor and/or salt thereof is administered at least eight times over a period of time. In embodiments, the phosphodiesterase type 5 inhibitor and/or salt thereof is administered at least ten times over a period of time.

In embodiments, the period of time is about 1 week to about 12 weeks. In embodiments, the period of time is about 1 week to about 10 weeks. In embodiments, the period of time is about 1 week to about 8 weeks. In embodiments, the period of time is about 1 week to about 6 weeks. In embodiments, the period of time is about 1 week to about 4 weeks. In embodiments, the period of time is about 2 weeks to about 12 weeks. In embodiments, the period of time is about 2 weeks to about 10 weeks. In embodiments, the period of time is about 2 weeks to about 8 weeks. In embodiments, the period of time is about 2 weeks to about 6 weeks. In embodiments, the period of time is about 2 weeks to about 4 weeks. In embodiments, the period of time is about 1 week. In embodiments, the period of time is about 2 weeks. In embodiments, the period of time is about 3 weeks. In embodiments, the period of time is about 4 weeks. In embodiments, the period of time is about 5 weeks. In embodiments, the period of time is about 6 weeks. In embodiments, the period of time is about 7 weeks. In embodiments, the period of time is about 8 weeks. In embodiments, the period of time is about 9 weeks. In embodiments, the period of time is about 10 weeks. In embodiments, the period of time is about 11 weeks. In embodiments, the period of time is about 12 weeks. In embodiments, the period of time is more than about 12 weeks.

In embodiments, one or more symptoms is improved within about 1 week to about 12 weeks of the initial administration. In embodiments, one or more symptoms is improved within about 1 week to about 10 weeks of the initial administration. In embodiments, one or more symptoms is improved within about 1 week to about 8 weeks of an initial administration. In embodiments, one or more symptoms is improved within about 1 week to about 6 weeks of the initial administration. In embodiments, one or more symptoms is improved within about 1 week to about 4 weeks of the initial administration. In embodiments, one or more symptoms is improved within about 1 week of the initial administration. In embodiments, one or more symptoms is improved within about 2 weeks of the initial administration. In embodiments, one or more symptoms is improved within about 3 weeks of the initial administration. In embodiments, one or more symptoms is improved within about 4 weeks of the initial administration. In embodiments, one or more symptoms is improved within about 5 weeks of the initial administration. In embodiments, one or more symptoms is improved within about 6 weeks of the initial administration. In embodiments, one or more symptoms is improved within about 7 weeks of the initial administration. In embodiments, one or more symptoms is improved within about 8 weeks of the initial administration. In embodiments, one or more symptoms is improved within about 10 weeks of the initial administration. In embodiments, one or more symptoms is improved within about 12 weeks of the initial administration.

In embodiments, the one or more symptoms comprises arousal lubrication. In embodiments, the one or more symptoms comprises sexual desire. In embodiments, the one or more symptoms comprises achieval of orgasm. In embodiments, the one or more symptoms comprises pleasure of orgasm.

In some embodiments, a female subject exhibiting one or more symptom of FSAD has not been administered at least one of guanylate cyclase stimulators, clonidine, CYP3A4 inhibitors, nitric oxide donors, organic nitrates, organic nitrites, or alpha blockers within about 28 days prior to the administration of a first dose of the composition. In some embodiments, a female subject exhibiting one or more symptom of FSAD has not been administered at least one of guanylate cyclase stimulators, clonidine, CYP3A4 inhibitors, nitric oxide donors, organic nitrates, organic nitrites, or alpha blockers within about 5, about 10, about 15, about 20, about 25, about 28, about 29, about 30, about 31, about 32, about 35, about 40, about 45, or about 50 days prior to the administration of a first dose of the composition.

In embodiments, the subject does not have history of active peptic ulcers. In embodiments, the subject does not have history of clinically significant bleeding disorders. In embodiments, the subject does not have history of clitoral priapism. In embodiments, the subject does not have history of a condition which may predispose them to clitoral priapism (such as sickle cell anemia, multiple myeloma, or leukemia). In embodiments, the subject does not have history of myocardial infarction. In embodiments, the subject does not have history of stroke. In embodiments, the subject does not have history of life-threatening arrhythmia. In embodiments, the subject does not have resting hypotension (BP<90/50 mmHg). In embodiments, the subject does not have history of coronary disease causing angina. In embodiments, the subject does not have history of congestive heart failure requiring medical intervention. In embodiments, the subject does not have history of an underlying condition which can be particularly sensitive to the actions of vasodilators, e.g. left ventricular outflow obstruction (e.g., aortic stenosis, idiopathic hypertrophic subaortic stenosis) or impaired autonomic control of blood pressure. In embodiments, the subject does not have history of hearing loss. In embodiments, the subject does not have retinitis pigmentosa, n embodiments, the subject does not have history of orthostatic hypotension or orthostatic hypotension (e.g., a drop in systolic blood pressure≥20 mm Hg, a drop in diastolic blood pressure≥10 mm Hg, an increase in pulse by 20 beats per minute or experiencing lightheadedness or dizziness at 1 or 3 minutes after the change in position from supine to standing).

In embodiments, the subject's sexual partner does not have a history of disease or disorder that may be contraindicated by the composition. In embodiments, the subject's sexual partner does not have a history of myocardial infarction. In embodiments, the subject's sexual partner does not have a history of stroke. In embodiments, the subject's sexual partner does not have a history of life-threatening arrhythmia. In embodiments, the subject's sexual partner does not have a history of resting hypotension (BP<90/50 mmHg). In embodiments, the subject's sexual partner does not have a history of coronary disease causing angina. In embodiments, the subject's sexual partner does not have a history of congestive heart failure requiring medical intervention In embodiments, the subject's sexual partner does not have a history of underlying conditions which can be particularly sensitive to the actions of vasodilators, e.g. left ventricular outflow obstruction (e.g., aortic stenosis, idiopathic hypertrophic subaortic stenosis). In embodiments, the subject's sexual partner does not have a history of impaired autonomic control of blood pressure. In embodiments, the subject's sexual partner does not have a history of orthostatic hypotension (e.g., a drop in systolic blood pressure≥20 mm Hg, a drop in diastolic blood pressure≥10 mm Hg, an increase in pulse by 20 beats per minute or experiencing lightheadedness or dizziness at 1 or 3 minutes after the change in position from supine to standing). In embodiments, the subject's sexual partner does not have a history of priapism or conditions which may predispose them to priapism (such as sickle cell anemia, multiple myeloma, or leukemia). In embodiments, the subject's sexual partner does not have a history of non-arteritic ischemic optic neuropathy (NAION) or any underlying NAION risk factors.

### Definitions

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

All definitions, as defined and used herein, should be understood to control over dictionary definitions, definitions in documents incorporated by reference, and/or ordinary meanings of the defined terms.

Terms such as “treat,” “treatment,” “treating,” etc. comprise therapeutic treatment of subjects having already developed a disease or disorder, in particular in manifest form. Therapeutic treatment may be symptomatic treatment in order to relieve the signs and/or symptoms of the disease or disorder or causal treatment in order to reverse, partially reverse, stop, or slow down the progression of the disease or disorder. Thus, the compositions and methods of the present disclosure may be used, for instance, as therapeutic treatment (e.g., for acute or chronic therapy).

Additionally, terms such as “prevent,” “preventing,” or “prevention” generally refer to the reduction of the occurrence of the disease or disorder, and/or a sign and/or symptom thereof, in the treated sample relative to an untreated control sample, or delays the onset of one or more signs and/or symptoms of the disease or disorder relative to the untreated control sample, in a statistically significant manner. Preventing the disease or disorder, and/or a sign and/or a symptom thereof, includes preventing or delaying the initiation of the disease, disorder, sign, and/or symptom. Prevention also includes preventing a recurrence of the disease, disorder, sign, and/or symptom.

In certain aspects, the composition can be applied to a subject, e.g., to clitoris, vestibule, vulva, and/or the vagina of a subject, and/or to another body cavity, for example, the mouth or the rectum. Any suitable technique may be used to apply the composition to the subject. For instance, the composition may be free or mass flowing, e.g., so that it may be administered through an applicator or other suitable device. Thus, in some embodiments, the composition may be contained within applicator, such as a vaginal applicator or a syringe, which can be applied, e.g., by the subject, or by another person.

The subject may be, but is not limited to, humans.

The phrase “genital area of a female subject” as used herein refers to the internal and external female sex organs. These include, without limitation, the vulva and vagina.

The term “vulva” includes all of the structures that make the female external genitalia. The components of the vulva include the mons pubis, labia majora, labia minora, clitoris, vestibular bulbs, vulva vestibule, Bartholin's glands, Skene's glands, urethra, and vaginal opening.

The term “vagina” refers to the elastic, muscular part of the female genital tract. In humans, the vagina extends from the vestibule to the cervix.

In one set of embodiments, as discussed, the composition is applied to treat the subject with a therapeutically effective amount of an active ingredient, such as any of those described herein. The therapeutically effective amount may be an amount which, when administered to a subject for treating or preventing a disease or disorder, is sufficient to effect such treatment or prevention for the disease or disorder, for example, any of those described herein. The therapeutically effective amount may also be an amount sufficient to elicit a desired biological response, i.e., alleviating a symptom. The therapeutically effective amount may vary depending on such factors as the desired biological endpoint, the mode of administration, and/or the age and health of the subject.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The term “about” when used before a numerical designation, e.g., temperature, time, amount, concentration, and such other, including a range, indicates approximations which may vary by (+) or (−) 10%, 5%, 1%, or any subrange or subvalue there between. Preferably, the term “about” when used with regard to an amount means that the amount may vary by +/−10%. When the word “about” is used herein in reference to a number, it should be understood that still another embodiment of the disclosure includes that number not modified by the presence of the word “about.”

When ranges are given by specifying the lower end of a range separately from the upper end of the range, it will be understood that the range can be defined by selectively combining any one of the lower end variables with any one of the upper end variables that is mathematically possible. Where ranges are recited, it will be understood that any subrange or value within the recited ranges, including endpoints, is contemplated.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

### List of Abbreviations

### Abbreviation Definition

AE Adverse event
CD Cognitive Debrief
CE Concept Elicitation
COS Clinical Outcomes Solutions
DHIF Demographic Health Information Form
FDA Food and Drug Administration
FSAD Female Sexual Arousal Disorder
FSDS-DAO Female Sexual Distress Scale-Desire, Arousal, Orgasm
FSDS-R Female Sexual Distress Scale-Revised
FSEP Female Sexual Encounter Profile
FSFI Female Sexual Function Index
GAS Genital Arousal Screener
GSM Genitourinary Syndrome Menopause
HRQL Health-related quality of life
HSDD Hypoactive Sexual Desire Disorder
ICF Informed Consent Form
IRB Institutional Review Board
MHF Medical History Form
N Number (uppercase is total population; lowercase is subgroup population)
PRO Patient Reported Outcome
SAE Serious adverse event
SAS Statistical Analysis System
SD Standard deviation
SFQ-28 Sexual Function Questionnaire-28
SOP Standard operating procedure
API Active pharmaceutical ingredient
BMI Body mass index
BP Blood pressure
CFR Code of Federal Regulations
CGMP Cyclic guanosine monophosphate
CRO Contract Research Organization
CSR Clinical study report eCRF Electronic case report form
ECG Electrocardiogram
ED Erectile dysfunction
FSD Female Sexual Dysfunction
GCP Good Clinical Practice
GLP Good Laboratory Practice
HBsAg Hepatitis B surface antigen
HCV Hepatitis C virus
HDPE High density polyethylene
HED Human equivalent dose
HIPAA Health Insurance Portability and Accountability Act
HIV Human immunodeficiency virus
HPV Human papillomavirus
ICF Informed consent form
ICH International Conference on Harmonisation
IND Investigational New Drug
IP Investigational product
IRB Institutional Review Board
IRT Interactive Response Technology
MedDRA Medical Dictionary for Regulatory Activities
NF National Formulary
NO Nitric oxide
NOAEL No observed adverse effect level
OTC Over-the-counter
PANAS Positive and Negative Affect Schedule
PDE5 Phosphodiesterase type-5
PI Principal Investigator
PK Pharmacokinetic
QA Quality assurance
QC Quality control
SAE Serious adverse event
SHBG Sex Hormone-Binding Globulin
SOC System Organ Class
TEAE Treatment-emergent adverse event TMF Trial master file
USP United States Pharmacopeia
VPA Vaginal Pulse Amplitude
VPP Vaginal Photoplethysmograph
WHODDE WHO Drug Dictionary Enhanced

### EXAMPLES

The following examples are intended to illustrate certain embodiments of the present disclosure, but do not exemplify the full scope of the disclosure.

### Example 1: Content Validation of Patient Reported Outcome (PRO) Measures for Use in Female Sexual Arousal Disorder

### Study Design

Given the need to collect evidence of content validity of the Arousal Diary, SFQ-28, and FSDS-DAO, qualitative interviews were conducted with individuals with clinically diagnosed FSAD.

Prior to Visit 1, initial eligibility was assessed. If the participant was determined to be potentially eligible, informed consent was collected. Following consent, the inclusion/exclusion criteria was confirmed, and a clinical interview was performed. The clinical interview was used to collect additional information and confirm a diagnosis of primary FSAD (N=35; n=20 pre-menopausal women; n=15 post-menopausal women). At Visit 2, the participant underwent an in-depth, qualitative, one-to-one interview, consisting of a CE and a CD discussion, lasting approximately 90 minutes in total.

The CE and CD interview at Visit 2 consisted of three components:
1. The first part of the interview involved CE discussion to explore participants' experience with FSAD and is expected to last approximately 45 minutes;
2. The second part involved participants completing three questionnaires; and
3 After each questionnaire was completed, the participant completed a debrief of several of the items on the questionnaires. The debriefing was expected to last approximately 45 minutes.

No drug intervention or treatment was administered as part of this study.

### Study Population

Up to 50 in-depth one-to-one, face-to-face qualitative interviews, involving a CE and CD discussion, were conducted; at least 20 with pre-menopausal women and at least 15 with post-menopausal women. Up to 50 women were enrolled to account for the possibility that some women may be excluded from the final analysis based on answers to interview questions during Visit 2 which indicate they do not meet the diagnostic criteria for FSAD. However, qualitative interviews were conducted to the point of saturation, which means that no new concepts are emerging. This was evaluated for both the pre-menopausal women in groups of seven, seven, and six interviews and for post-menopausal women in groups of five interviews. A saturation matrix was developed to document this. If, after the 20 interviews in the pre-menopausal group and 15 interviews in the post-menopausal cohort, new concepts are still being discussed, further interviews may be recommended in that cohort.

### Inclusion Criteria

Each potential participant must satisfy all the following criteria to be enrolled in the study.
1. Patient must be between the ages of 21 and 70 years, inclusive.
2. Patient is capable of understanding and complying with the protocol and agrees to sign the informed consent document.
3. Patient has been sexually active (in the past 4 weeks). Sexual activity can include any activity which may result in sexual stimulation or sexual pleasure e.g. intercourse, caressing, foreplay, masturbation, and oral sex.
4. Based on a clinical interview, patient has previously experienced ‘normal’ sexual function for at least 2 years or longer (i.e. normal sexual desire, arousal, orgasm, no pain).
5. Post-menopausal women (surgically induced or natural) must meet 1 of the following criteria:
   a. Bilateral oophorectomy, with or without hysterectomy, at least 1 year prior to screening (self-report).
   b. 12 months of spontaneous amenorrhea
   c. 6 months of spontaneous amenorrhea with serum FSH levels>40 mIU/mL (except for those women on hormone replacement therapy)
6. Patient has a body mass index (BMI) from 18 to 33 kg/m², inclusive.
7. Patient is fluent in the English language.
8. Patient agrees to not use cannabinoid-containing products within 24 hours of Visit 1 and Visit 2.

### Exclusion Criteria

Any potential participant who meets any of the following criteria was excluded from participating in the study:
1. Patient has any disorder or a history of any disorder that may prevent the successful completion of the study in the opinion of the Investigator.
2. Patient self-reports or has a current diagnosis of an uncontrolled medical condition (e.g., cardiovascular, hepatic, metabolic, renal, respiratory, gastrointestinal, endocrine, immunologic, dermatologic, hematologic, neurologic, genitourinary, or psychiatric).
3 Patient reports a history of sexual trauma or abuse that is contributing to any sexual dysfunction problem (desire, arousal, orgasm, pain etc.) as determined by the clinical interview.
4. Patient has a history of myocardial infarction, stroke, or life-threatening arrhythmia within 6 months prior to the clinical interview or any history of coronary disease causing angina; or congestive heart failure requiring medical intervention.
5. Patient has a primary complaint of anorgasmia, pain, vaginismus, low desire, or any other primary sexual complaint aside from problems with genital arousal, as determined by the clinical interview.
6. Patient is symptomatic or has a current diagnosis of dyspareunia, vulvovaginal infection or inflammation, inflammatory disorders of the vulva or vagina, vestibulodynia, clitorodynia, or vulvovaginal atrophy (patients who present with vaginal dryness without pain as their only symptom of vulvovaginal atrophy will remain eligible) or is symptomatic for any other vulvar or vaginal disorder as determined by the clinical interview.
   a. Patients with bacterial vaginosis or a yeast infection can be rescreened once the infection is treated and has resolved.
7. Patient has undergone major pelvic surgery that may have caused nerve damage, including but not limited to vulvectomy, colostomy, cystostomy, or significant bladder (including for incontinence), rectal, or abdominal surgery.
8 Patient reports or has been diagnosed with neurological impairment due to conditions such as diabetes, stroke, pelvic nerve damage secondary to trauma, cancer treatments, myasthenia gravis, multiple sclerosis, spinal cord damage etc.
   a. Patients who are symptomatic of peripheral neuropathy will be excluded, including those patients who report ‘numbness’ or ‘no feeling’ in the genitals during sexual activity.
9. With the exception of anxiety and depression, participant has any current and/or previous reported diagnoses of DSM-IV-TR axis I disorders including organic mental syndromes and disorders.
   a. Patients diagnosed with anxiety or depression must be controlled, as determined by the Investigator; and if on medication on a stable medication and dose for at least 6 months prior to the clinical interview.
   b. Patients have a total score<10 indicating minimal depression or mild depression on the Patient Health Questionnaire-8 (a screening and assessment instrument for depression).
   c. Patients have a total score<6 indicating mild anxiety on the Generalized Anxiety Disorder (GAD)-7 (a screening and assessment instrument for anxiety).
10. Patient has a history of gynecological cancer or is under active treatment for any cancer which would interfere with the patient's ability to successfully complete the study. Patients who have been previously treated for dysplasia (precancerous changes) may be included as long as the patient received localized treatment (e.g., cryosurgery or laser).
11. Patient has a history of drug abuse within 1 year prior to Visit 1. Patients who report a history of drug abuse in the past must be in remission or sober for at least one year prior to the clinical interview.
12. Patient has a history of alcoholism within 1 year prior to Visit 1. Patients who report a history of alcoholism in the past must be in remission or sober for at least one year prior to the clinical interview.
13. Patient is currently receiving treatment, or has received treatment within the last three months, for FSAD symptoms (pharmacologic or non-pharmacologic).
14. Patient has positive findings from the urine drug screen (e.g., amphetamines, barbiturates, benzodiazepines, cocaine, methadone, and opiates) unless for a known disorder (e.g., ADHD) which would not interfere with the patient's ability to successfully complete the study.
15. Patient is symptomatic or has been diagnosed with chlamydia, gonorrhea, or syphilis in the past 3 months.
16. Patient reports having an HPV or HSV genital or anal outbreak (blisters, warts or vesicles) at any time point in the past month. Patient can be rescreened once the outbreak is treated and has resolved. Patients who report genital skin breaks, irritation, dermatoses, or lesions or any other abnormal vulvar-vaginal findings will also be excluded.
17. Patient has participated in any clinical research study evaluating another investigational drug or therapy within 3 months before Visit 1.
18. Patient has been exposed to any of the measures under investigation, including the Arousal Diary, Sexual Function Questionnaire (SFQ), Female Sexual Distress Scale-Desire, and Arousal, Orgasm (FSDS-DAO), or other sexual dysfunction questionnaires (e.g., FSFI, FSDS-R, FSEP) in the past 6 months before the clinical interview.
19. Patient is symptomatic or currently diagnosed with a pelvic or urinary tract infection. Patient can be rescreened once the infection is treated and has resolved.
20. Patient self-reports she is nursing or pregnant, plans to begin nursing during the study period or was pregnant or nursing within 6 months prior to Visit 1.

### Clinical Diagnosis of FSAD

Patients who meet all of the above inclusion and exclusion criteria underwent a formal, semi-structured clinical interview at Visit 1 to confirm diagnosis of primary FSAD as defined by the DSM-IV-TR.

Primary FSAD is defined by the DSM-IV-TR which is acquired (i.e., not lifelong) and generalized (i.e., present regardless of contextual setting) and with symptoms which have been present for at least the past 6 months prior to the clinical interview. Eligible patients must have a primary complaint of lack of genital arousal during sexual activity and regard their genital response as meaningful to their overall sexual experience. Eligible patients must also be experiencing distress because of their sexual arousal difficulties. Patients who have secondary complaints related to low desire due to lack of genital arousal [Hypoactive Sexual Desire Disorder] were considered eligible as long as problems related to diminished genital arousal are deemed the most bothersome symptom and developed before low desire. Patients who present with cognitive arousal problems (i.e. not feeling aroused mentally during sexual activity) were also considered eligible as long as problems related to diminished genital arousal are deemed the most the bothersome symptom and developed before cognitive arousal problems.

As FSAD is a diagnosis of exclusion, the clinical interview also focused on excluding patients who are symptomatic for vulvar or vaginal disorders including but not limited to vulvovaginal infections or inflammation, dyspareunia, Genitourinary Syndrome of Menopause (GSM) or vulvovaginal atrophy, inflammatory disorders of the vulva or vagina (lichen sclerosus, lichen planus, plasma cell vulvitis, mucous membrane pemphigoid, desquamative inflammatory vaginitis, Sjogren's syndrome, contact dermatitis, allergic vaginitis, vestibulodynia, and clitorodynia).

### Recruitment and Screening

### Recruitment Strategy

Clinics were female sexual health specialty centers or medical facilities. Recruitment remained open until study enrolment goals (N=35; n=20 pre-menopausal, n=15 post-menopausal women) had been met. Screen failures were not counted toward the total site enrolment.

All recruitment procedures complied with the Institutional Review Board (IRB)-approved protocol. Potential participants were scheduled for a Visit 1 screening appointment.

Participants are free to withdraw from the study for any reason and may revoke/cancel authorization/withdraw from participation at any time by providing written notice and without penalty or prejudice. A participant's involvement may be terminated in the study at any time if the participant's clinical condition warrants it.

The specific objectives are:
Stage 1: Concept elicitation (CE) interviews to:
   Explore the experience of disease-associated symptoms and the relative importance of FSAD symptoms including their severity and bother and impact on patients' health-related quality of life (HRQL)
Stage 2: Cognitive debriefing (CD) interviews to:
   Explore the relevance of the selected PRO measures based on patients' own experiences
   Determine patients' understanding of the items, instructions, and response options of the selected PRO measures
   Assess the appropriateness of the specified recall periods of the selected PRO measures, with a focus on 24-hour recall period vs 4 weeks.

### Screening Procedures (Visit 1)

The participant were also asked to undergo a semi-structured clinical interview where a diagnosis of primary FSAD was confirmed. Once eligibility was confirmed participants were scheduled for Visit 2, the CE and CD interview.

### Sampling Stratification

### Interview Procedures (Visit 2)

Interviews at Visit 2 were conducted either face-to-face or via telephone, if face-to-face was not possible. The CE portion of the interview was first and lasted approximately 45 minutes. Participants were then be asked to fill out the Arousal Diary, SFQ-28, and the FSDS-DAO, which took approximately 10 minutes. Following this, the CD part of the interview commenced and lasted approximately 45 minutes.

### Concept Elicitation

Face-to-face qualitative interviews began with a brief CE set of questions with each participant. This will take the form of an open discussion in which each participant discussed her individual experience of sexual arousal problems and any associated impact. This was used to obtain a full and individual-led picture of the impact of FSAD to ensure adequate coverage of the key arousal problems experienced and its impact. After this, participants talked about the severity of the elicited symptoms and ranked their most bothersome symptoms and the symptoms they feel are most important for treating. Participants also discussed the emotional impact of FSAD to determine what language women use spontaneously to describe this e.g. bother, distress, frustration.

### Cognitive Debriefing

Following the CE portion, each of the participants then filled out each of the questionnaires. The purpose of the CD interview was to explore participants' understanding and relevance of the measures. Participants first completed each measure as they would in a clinical trial (i.e. without interruption from the interviewer). Participants were queried on the relevance and their experience with the concepts assessed by each item. For items with concepts that participants state as relevant and/or that they've experienced, additional questions to assess item content, response options, and scoring were be asked to further debrief the item. In order to test whether there was an order effect in women's preference for items based on exposure (i.e. a preference for those questions they are exposed to first), the order of the questionnaires was modified. Half of the pre-menopausal women and the first eight post-menopausal women received the questionnaires in this order: SFQ-28, FSDS-DAO, and Arousal Diary; the other half of the pre-menopausal women and seven of the post-menopausal women received them in this order: Arousal Diary, SFQ-28, and FSDS-DAO.

The interview also discussed any “new” issues mentioned during the CE part of the interview that were not captured within the measures to explore whether participants feel these “new” concepts should be added, or whether the content of the measures allows them to describe these experiences adequately.

### Analysis

### Participant Characteristics

Participant characteristics were summarized to characterize the enrolled population and provided context to the qualitative data. Continuous data were summarized as number (n), mean, and standard deviation (SD). Categorical data were summarized in terms of the number of participants (n) providing data at the relevant time point, frequency counts, and percentages. Percentages were based on the participant with a non-missing parameter. Percentages were reported to two decimal places. Analyses was performed using Statistical Analysis System (SAS) v9.4.

### Qualitative Data Analysis

### Analysis of CE Data

The transcribed data was entered into NVivo v.12, a software package that is designed to facilitate the storage, coding and analysis of qualitative data. This software allows for themes, concepts, or domains to be grouped by different variables (e.g., ethnicity, age). Analysis of CE interview data was guided by the objectives of the study and key concepts of interest and thematically coded. A preliminary codebook based on study objectives and key concepts of interest was developed. Using this codebook, the interview transcripts were coded and new codes that emerge were identified in subsequent interviews. Revisions to the coding structure were monitored to ensure that all codes are clearly defined and consistently applied.

### Saturation Analysis

To support the validity of the selected PRO measures, it was important to demonstrate that all important concepts had emerged in the interview sample, referred to as ‘saturation analysis’. Concept saturation was assessed for the CE interviews, and that no new themes or descriptions of concepts were being introduced upon the final interview. Concept elicitation interviews were conducted to the point of saturation (when there are no new concepts emerging from subsequent interviews), and so additional interviews were potentially conducted until this was achieved.

Saturation analysis was only undertaken on the CE portion of the interviews since CD is confirmatory by nature. Saturation analysis is appropriate only when the interview is exploratory by intent (i.e., CE interviews). To determine this, CE interview transcripts (N=35) were separated into pre-menopausal and post-menopausal women. Pre-menopausal interview transcripts were grouped into three sets (n=7, n=7, and n=6 in each set) and post-menopausal interview transcripts were grouped into three sets of n=5, in the sequential order they were performed. Concepts elicited were then compared between sets in each group. The process for determining if conceptual saturation had been achieved among adult participants was as follows:
Thematically-derived concepts were converted into two ordinal groups: “not discussed” equals 0, “discussed” equals 1.
The concepts that emerged in the first set of interviews were compared with the concepts that emerged from the second set of interviews.
The concepts that emerged from these first two sets were then compared with the concepts that emerged from the third set of interviews.
The point at which no new concepts emerged is the point at which saturation was deemed to have been achieved.

If saturation had not been achieved once all interviews had been analyzed, then recommendations were developed regarding the utility of conducting additional interviews.

### Analysis of CD Data

Analysis of CD data focused on quotes that pertain to the main research questions: including relevance, comprehension, and any rewording suggestions. The first two CD transcripts were coded to develop a preliminary codebook for the CD interviews. Using this codebook, the remaining interview transcripts were coded to identify any new codes that emerged in subsequent interviews. Revisions to the coding structure were monitored to ensure that all codes were clearly defined and consistently applied.

### Example 2: a Phase 2b, Multi-Center, Multiple-Dose, Randomized, Double-Blind Placebo-Controlled Study to Evaluate the Efficacy and Safety of 3.6% Sildenafil Cream in Premenopausal Patients with Female Sexual Arousal Disorder (FSAD)

There are currently no approved treatments for female sexual arousal disorder (FSAD. An objective of this study was to test the systemic and local genital safety of topical Sildenafil Cream, 3.6% among healthy premenopausal women with FSAD and their sexual partners over a 12-week treatment period.

To assess patient-reported outcomes (PROs) among women with FSAD, several PRO measures, including the Sexual Function Questionnaire (SFQ28), Female Sexual Distress Scale-Desire, Arousal, Orgasm (FSDS-DAO), Patient Global Impression of Severity (PGI-S), Patient Global Impression of Change (PGI-C), Patient Global Impression of Frequency (PGI-F), Patient Global Impression of Change with Concern with Sexual Difficulties (PGI-C Concern), Patient Global Impression of Change in Satisfactory Sexual Events (PGI-C SSE), and the Arousal Diary, were used to evaluate change in female sexual function following the administration of Sildenafil Cream, 3.6%, in the double-blind, placebo-controlled phase 2b study.

Female sexual arousal disorder (FSAD) is defined in the Diagnostic and Statistical Manual of Mental Disorders Text Revision Fourth Edition (DSM-IV-TR) as a persistent or recurrent inability to attain, or to maintain until completion of the sexual activity, an adequate lubrication-swelling response of sexual excitement that causes marked distress or interpersonal difficulty. FSAD is estimated to negatively impact approximately 20% of women in the United States (US), and disruptions in sexual arousal impact other aspects of sexual response. Orgasm is less possible without arousal and FSAD commonly leads to a lack of desire because sexual activity is not enjoyable or reinforcing. Despite the high prevalence of FSAD and the potential impact it has on other aspects of sexual function in women, to date there are no US Food and Drug Administration (FDA) approved pharmacological treatments for FSAD. Most commonly, treatments involve the administration of topical lubricants that help to mask the lack of vaginal lubrication associated with FSAD, but are ineffective in enhancing genital/clitoral blood flow or alleviating the decrease in genital sensations that accompany FSAD.

Published placebo-controlled trials of oral sildenafil citrate administered in pre- and postmenopausal women afflicted with broad-spectrum FSD (e.g., Hypoactive Sexual Desire Disorder (HSDD), FSAD, Female Orgasmic Disorder (FOD), Dyspareunia) unrelated to concomitant disease or substance use demonstrated marginal efficacy versus placebo.

The present disclosure provides a topical Sildenafil Cream, 3.6% for the treatment of FSAD. By delivering sildenafil topically, specifically targeting genital anatomy central to the vascular arousal response, there will be less systemic exposure, followed by less systemic side effects, resulting in a more favorable safety profile. The co-primary objectives of this study were to evaluate the safety and efficacy of Sildenafil Cream, 3.6% among healthy premenopausal women with FSAD.

### Patient Evaluation

Healthy premenopausal women aged 18 years-old or older and their sexual partners were screened. Volunteers and their sexual partners provided written informed consent prior to the performance of any study related procedures. Women at risk of pregnancy used effective contraception during the study. Women or their partners were excluded if they had uncontrolled hypertension, a history of serious cardiac events (e.g., myocardial infarction, stroke), orthostatic hypotension or other serious medical co-morbidities. A one-on-one individual clinical interview was conducted with each potential participant, using interviewers who were not employees of the study sponsors and who were recognized experts in the field, in order to establish the diagnosis of FSAD and verify that FSAD was the woman's primary sexual dysfunction concern if other secondary FSDs existed. Table 2 outlines the schedule of evaluations.

Volunteers and their sexual partners underwent informed consent and screening safety procedures at visit 1. Approximately 1 year into the study, to allow participation from women for whom a barrier to recruitment was the requirement for sexual partners to be consented and involved in adverse event (AE) reporting, as many volunteers did not want to disclose their FSAD symptoms to their sexual partners, the protocol was amended. Specifically, amendment 3 of the protocol, allowed for women to enroll in the trial if they did not have a sexual partner, or if their sexual partner did not want to participate in the informed consent and safety monitoring process. In the latter case, investigational product (IP) was only used for un-partnered, solo sexual experiences, and if the subject had a sexual partner, no partnered sexual events could occur within 72 hours of IP application. If a partnered sexual experience was recorded with an un-consented partner, the participant was discontinued from the study.

The study included a 28-day no drug run-in period, followed by a 28-day single-blind placebo run-in period. Participants were screen failed during these 2 months if they were not compliant with recording sexual experiences and AEs in the electronic diary (eDiary), or if they had a pre-determined placebo response on the female sexual distress scale-desire arousal and orgasm (FSDS-DAO) during the single-blind placebo run-in period (total score≤18). Eligible participants were randomized at visit 4 in a 1:1 manner to Sildenafil Cream, 3.6% versus Placebo Cream.

The double-blind treatment period lasted 12 weeks, from visit 4 to visit 7. Participants were seen monthly during this period and treatment emergent adverse events (TEAEs), and the severity and the relationship of TEAEs to study product or study procedures were determined by the investigator. In between visits, participants recorded TEAEs using the eDiary.

### Patient and Partner—Assessment for Study Participation and Safety

Eligible patients and their respective partner completed a separate Informed Consent Form (ICF) prior to participating in the study at Visit 1. Both the patient and her partner also met all the respective inclusion and exclusion criteria required for participation in the study assessed at Visit 1. Both the patient and the partner completed assessments, as applicable, and recorded any adverse events at home using separate eDiaries. In addition, the eDiary was used to complete the PRO assessments (SFQ28, FSDS-DAO, PGI-S, PGI-C and PBE) by the patient at scheduled clinic visits.

The eDiary prompted the patient to complete the eDiary within 24 hours of each sexual event and at any time during the course of the study when an AE occurs. If a sexual event occurred the patient used the eDiary to record the application of IP, record any adverse events and complete the Arousal Diary. The eDiary also prompted the partner every 24 hours to assess whether there has been a partnered sexual event(s) for the purpose of collecting any adverse events.

If there was a sexual event, the eDiary provided the following prompt to the patient and the partner: ‘Since your sexual event, do you have any complaints?’ If the patient or partner answers ‘YES’ the eDiary further prompted them for some of the more common side effects associated with oral phosphodiesterase type 5 (PDE5) inhibitors. Assuming wireless connectivity, the responses from patients or partners who report a complaint following sexual activity were sent electronically to the site for evaluation and triage within 24 hours of receipt of the information. Additionally, patients and their partners that have answered “YES” to complaints were reminded to contact their study site.

### Number of Patients, Demographics, and Baseline Characteristics

Volunteers (n=833) and their sexual partners (n=605) underwent informed consent. Of the consented subjects, 277 eligible participants entered the no drug run-in period and 252 entered the single-blind placebo run-in period. Two hundred participants were randomized to either Sildenafil Cream, 3.6% (n=101) or Placebo Cream (n=99), and 99 Sildenafil Cream, 3.6% assigned women and 94 Placebo Cream assigned women received at least one dose of their assigned IP in the double-blind treatment period. There were no allocation errors. An additional 40 women, who were exposed to at least one dose of placebo cream during the single-blind run-in period, were also included in the safety analysis population, bringing the total to 134 placebo exposed participants. Table 1A displays the demographics and baseline characteristics of subjects included in the safety analysis population.

TABLE 1A
Demographic and Baseline Characteristics of Safety Population Participants and Sexual Partners

| | Participants | | Participants | | Participants | |
|---|---|---|---|---|---|---|
| | Sildenafil | | Placebo | | Total | |
| Participant Characteristic | (N = 99) | | (N = 134) | | (N = 233) | |
| Participant | | | | | | |
| Continuous Variables | Mean | SD | Mean | SD | Mean | SD |
| Age (Years) | 36.3 | 7.14 | 36.3 | 7.21 | 36.3 | 7.17 |
| Baseline Body | 27.1 | 4.7 | 27.2 | 4.6 | 27.2 | 4.6 |
| Mass Index (BMI) (kg/m²) | | | | | | |
| | | | | | | % Total |
| | | % Total | | % Total | | Study |
| Participant | | Product | | Product | | Safety |
| Categorical Variables | N | Group | N | Group | N | Population |
| Cis Gender Female | 99 | 100 | 134 | 100 | 233 | 100 |
| Ethnicity | | | | | | |
| Hispanic or Latino | 14 | 14.1 | 24 | 17.9 | 38 | 16.3 |
| Not Hispanic or | 85 | 85.9 | 110 | 82.1 | 195 | 83.7 |
| Latino | | | | | | |
| Race | | | | | | |
| American Indian or | 0 | 0 | 2 | 1.5 | 2 | 0.9 |
| Alaskan Native | | | | | | |
| Asian | 5 | 5.1 | 7 | 5.2 | 12 | 5.2 |
| Black or African | 6 | 6.1 | 14 | 10.4 | 20 | 8.6 |
| American | | | | | | |
| Native Hawaiian or | 1 | 1.0 | 0 | 0 | 1 | 0.4 |
| other Pacific | | | | | | |
| Islander | | | | | | |
| White | 84 | 84.8 | 110 | 82.1 | 194 | 83.3 |
| Mixed Race or | 3 | 3.0 | 1 | 0.7 | 4 | 1.7 |
| Other | | | | | | |
| Employment Status | | | | | | |
| Employed Full-Time | 73 | 73.7 | 97 | 72.4 | 170 | 73.0 |
| Employed Part-Time | 6 | 6.1 | 7 | 5.2 | 13 | 5.6 |
| Self-Employed | 5 | 5.1 | 4 | 3.0 | 9 | 3.9 |
| Student | 1 | 1.0 | 4 | 3.0 | 5 | 2.1 |
| Retired | 0 | 0 | 1 | 0.7 | 1 | 0.4 |
| Unemployed | 14 | 14.1 | 21 | 15.7 | 35 | 15.0 |
| Highest Level of Education | | | | | | |
| Some High School | 5 | 5.1 | 10 | 7.5 | 15 | 6.4 |
| or High School | | | | | | |
| Diploma/Certificate | | | | | | |
| Technical School, | 24 | 24.2 | 37 | 27.6 | 61 | 26.2 |
| Occupational | | | | | | |
| School, Associates | | | | | | |
| Degree and or | | | | | | |
| Some College | | | | | | |
| Bachelor's Degree | 40 | 40.4 | 43 | 32.1 | 83 | 35.6 |
| Master's or | 6 | 6.1 | 11 | 8.2 | 17 | 7.3 |
| Doctoral Degree | | | | | | |
| Missing | 0 | 0 | 1 | 0.7 | 1 | 0.4 |
| | Partners | | Partners | | Partners | |
| | Sildenafil | | Placebo | | Total | |
| Partner Characteristic | (N = 91) | | (N = 112) | | (N = 203) | |
| Partner Continuous | | | | | | |
| Variables | Mean | SD | Mean | SD | Mean | SD |
| Age (Years) | 38.5 | 8.60 | 37.6 | 8.48 | 38.0 | 8.52 |
| BMI (kg/m²) | 28.5 | 5.7 | 28.7 | 4.5 | 28.6 | 5.1 |
| | | | | | | % Total |
| | | % Total | | % Total | | Study |
| Partner Categorical | | Product | | Product | | Safety |
| Variables | N | Group | N | Group | N | Population |
| Sex | | | | | | |
| Female | 5 | 5.5 | 10 | 8.9 | 15 | 7.4 |
| Male | 86 | 94.5 | 102 | 91.1 | 188 | 92.6 |

### Primary Objectives:

SFQ28 (AS)—28 Day Recall: To evaluate the efficacy of Sildenafil Cream, 3.6% vs Placebo Cream in patients with FSAD as measured by change from baseline to the end of study (i.e., end of 12-Week Double-blind Dosing period) in the Arousal-Sensation Domain of the Sexual Function Questionnaire (SFQ28). The SFQ28 (AS) score at the end of the Single-Blind Placebo Run-In will serve as baseline and the Sildenafil Cream, 3.6% arm results will be compared with the Placebo Cream arm results.

The SFQ28 is a 28-item PRO questionnaire that addresses a majority of aspects of the sexual response cycle (desire, arousal, orgasm) as well as pain or discomfort. Specifically, the SFQ28 has 8 domains (Desire, Arousal Sensation, Arousal-Lubrication, Arousal-Cognitive, Orgasm, Pain, Enjoyment, and Partner) which asks about a woman's sexual activity and sexual life with their partner over the last 4 weeks. The SFQ28 defines sexual life as both physical sexual activities and the emotional sexual relationship that the person had with their partner.
FSDS-DAO (Q14)—28 Day Recall: To evaluate the efficacy of Sildenafil Cream, 3.6% vs Placebo Cream in patients with FSAD as measured by change from baseline to end of study in the score for feeling concerned by difficulties with sexual arousal—Item #14 only of the Female Sexual Distress Scale-Desire, Arousal, Orgasm (FSDS-DAO). The FSDS-DAO (Q14) score at the end of the Single-Blind Placebo Run-In will serve as baseline and the Sildenafil Cream, 3.6% arm results will be compared with the Placebo Cream arm results.

The FSDS-DAO is a 15-item PRO questionnaire that includes a list of feelings and problems that women sometimes have concerning their sexuality. Specifically, women are asked to rate each item in terms of frequency from 0 (never) to 4 (always) that best describes “how often that problem has bothered you or caused you distress during the past 30 days including today”. To calculate a total score on this measure, items are summed into a total ranging from 0 to 60, with higher scores indicating more sexually related distress. In this study, Item 14 is scored using the value associated with the selected response option.

### Secondary Objective:

Arousal Diary (SSE): To evaluate the efficacy of Sildenafil Cream, 3.6% in patients with FSAD as measured by change from baseline to end of study (i.e., last four weeks of Double-Blind Dosing Period) in number of Satisfactory Sexual Events (question #11 in the Arousal Diary) completed by daily prompt within 24 hours after each sexual event. The Arousal Diary (SSE) scores during the Single-Blind Placebo Run-In will serve as baseline and the Sildenafil Cream, 3.6% arm results will be compared with the Placebo Cream arm results.

### Exploratory Objectives:

Completed by eDiary (using a daily prompt) within 24 hours after each sexual event.
Arousal Diary (AS): To evaluate the efficacy of Sildenafil Cream, 3.6% in patients with FSAD as measured by change from baseline at Week 4 (Visit 5), Week 8 (Visit 6), and Week 12 (Visit 7) in the Arousal-Sensation domain of an adapted SFQ28 (questions #1-5 of the Arousal Diary) completed within 24 hours after each sexual event.

Arousal Diary questions include: “How much pleasurable sensation in your vagina/genital area did you feel during sexual activity?” (Item 1), “How much ‘warmth’ (e.g., sensation of increased temperature) did you feel in your vagina/genital area during sexual activity?” (Item 2), “How much ‘pulsating’ or ‘throbbing’ did you feel in your vagina/genital area during sexual activity?” (Item 3), “How much ‘tingling’ in your vagina/genital area did you feel during sexual activity?” (Item 4), and “How much ‘engorgement’ or ‘fullness’ did you feel in your vagina/genital area during sexual activity?” (Item 5). Responses range on a 5-point scale from 1 (no pleasurable sensation/no feeling of “warmth”/no “pulsating” or “throbbing” sensation/no “tingling” sensation/no feeling of “engorgement” or “fullness,” respectively) to 5 (a very strong pleasurable sensation/extremely “warm”/a very strong “pulsating” or “throbbing” sensation/a very strong “tingling” sensation/a very strong feeling of “engorgement” or “fullness,” respectively).
Arousal Diary (AL): To evaluate the efficacy of Sildenafil Cream, 3.6% in patients with FSAD as measured by change from baseline at Week 4 (Visit 5), Week 8 (Visit 6), and Week 12 (Visit 7) in the Arousal-Lubrication domain of an adapted SFQ28 (question #6 of the Arousal Diary) completed within 24 hours after each sexual event.
Arousal Diary (GA): To evaluate the efficacy of Sildenafil Cream, 3.6% in patients with FSAD as measured by change from baseline at Week 4 (Visit 5), Week 8 (Visit 6), and Week 12 (Visit 7) in the Genital Arousal questions (#7-9 of the Arousal Diary) completed within 24 hours of each sexual event.
Arousal Diary (SSE): In addition to secondary objective described above, evaluate the efficacy of Sildenafil Cream, 3.6% in patients with FSAD as measured by change from baseline to Week 4 (Visit 5) and Week 8 (Visit 6) in number of Satisfactory Sexual Events (question #11 in the Arousal Diary) completed by daily prompt.

Completed at the end of the No Drug Run-In, the Single-Blind Placebo Run-in, and after each month during the Double-Blind Dosing Period.
SFQ28 (AS)—28 Day Recall: In addition to the secondary objective described above, evaluate the efficacy of Sildenafil Cream, 3.6% in patients with FSAD as measured by change from baseline in the Arousal-Sensation domain at Week 4 (Visit 5) and Week 8 (Visit 6).
FSDS-DAO (Q14)—28 Day Recall: In addition to the secondary objective described above, evaluate the efficacy of Sildenafil Cream, 3.6% in patients with FSAD as measured by change from baseline in FSDS-DAO Item 14 at Week 4 (Visit 5) and Week 8 (Visit 6).
SFQ28 (AL)—28 Day Recall: Evaluate the efficacy of Sildenafil Cream, 3.6% in patients with FSAD as measured by change from baseline in the Arousal-Lubrication domain at Week 4 (Visit 5), Week 8 (Visit 6) and Week 12 (Visit 7).
SFQ28 (Orgasm)—28 Day Recall: Evaluate the efficacy of Sildenafil Cream, 3.6% in patients with FSAD as measured by change from baseline in the Orgasm domain at Week 4 (Visit 5), Week 8 (Visit 6) and Week 12 (Visit 7).
SFQ28 (Arousal Cognitive)—28 Day Recall: Evaluate the efficacy of Sildenafil Cream, 3.6% in patients with FSAD as measured by change from baseline in the Arousal Cognitive domain at Week 4 (Visit 5), Week 8 (Visit 6) and Week 12 (Visit 7).
SFQ28 (Desire)—28 Day Recall: Evaluate the efficacy of Sildenafil Cream, 3.6% in patients with FSAD as measured by change from baseline in the Desire domain at Week 4 (Visit 5), Week 8 (Visit 6) and Week 12 (Visit 7).
FSDS-DAO—28 Day Recall: Evaluate the efficacy of Sildenafil Cream, 3.6% in patients with FSAD as measured by change from baseline in the total score at Week 4 (Visit 5), Week 8 (Visit 6) and Week 12 (Visit 7).
PGI-S: Evaluate the efficacy of Sildenafil Cream, 3.6% in patients with FSAD as measured by Patient Global Impression of Severity at Week 4 (Visit 5), Week 8 (Visit 6) and Week 12 (Visit 7).
PGI-C: Evaluate the efficacy of Sildenafil Cream, 3.6% in patients with FSAD as measured by Patient Global Impression of Change at Week 4 (Visit 5), Week 8 (Visit 6) and Week 12 (Visit 7) of the Double-Blind Dosing Period.

Completed at the end of the Double-Blind Dosing Period.
PBE (Patient Benefit Evaluation): Evaluate the efficacy of Sildenafil Cream, 3.6% in patients with FSAD as measured by PBE assessed at the end of double-blind treatment with a Yes/No question.

Table 1B shows an overview of PRO instruments and assessments used in the trial for clinical diagnosis of FSAD, inclusion criteria for FSAD, and primary, secondary, and exploratory endpoints.

TABLE 1B
Summary of PRO Instruments and Assessments for FSAD Diagnosis, FSAD
Eligibility Criteria, and Primary, Secondary and Exploratory Endpoints

| | | | | | End of | | |
|---|---|---|---|---|---|---|---|
| | | | End | End of | each month | | |
| | | | of No | Single-Blind | during the | | |
| | | | Drug | Placebo | Double Blind | | |
| | | Screening | Run-In | Run-In | Treatment Period | | |
| | PRO | (Visit 1) | (Visit 3) | (Visit 4) | (Visits 5, 6, and 7) | | |
| FSAD | Clinical | X | | | | | |
| Diagnosis | Interview | | | | | | |
| FSAD | | | | | | | |
| Eligibility | FSDS-DAO | X | X | | | | |
| | SFQ28 | X | X | | | | |
| Primary | SFQ28 (AS) | | | X^{b} | X | X | X |
| Endpoints | FSDS-DAO- | | | X^{b} | X | X | X |
| | Q14 | | | | | | |
| Secondary | Arousal Diary | | X^{a} | X^{a,b} | Xª | X | X^{a} |
| Endpoint | (SSE) | | | | | | |
| | FSDS-DAO | | | X^{b} | X | X | X |
| Exploratory | SFQ28 (Orgasm) | | | X^{b} | X | X | X |
| Endpoints | SFQ28 (Arousal | | | X^{b} | X | X | X |
| | Lubrication) | | | | | | |
| | SFQ28 (Arousal | | | X^{b} | X | X | X |
| | Cognitive) | | | | | | |
| | SFQ28 (Desire) | | | X^{b} | X | X | X |
| | PGI-S | | X | X^{b} | X | X | X |
| | PGI-C | | X | X^{b} | X | X | X |
| | PGI-F | | X | X^{b} | X | X | X |
| | PGI-C Concern | | X | X^{b} | X | X | X |
| | PGI-C SSE | | X | X^{b} | X | X | X |
| | PBE | | | | | | X |
| | Arousal Diary | | X^{a} | X^{a,b} | X^{a} | X^{a} | X^{a} |
| | (AL) | | | | | | |
| | Arousal Diary | | X^{a} | X^{a,b} | X^{a} | X^{a} | X^{a} |
| | (AS) | | | | | | |
| | Arousal Diary | | X^{a} | X^{a,b} | X^{a} | X^{a} | X^{a} |
| | (GA) | | | | | | |

Note:
no PRO instruments or assessments are administered at Visit 2.
^{a}Arousal Diary to be completed (by daily prompt) within 24 hours after completion of each sexual activity.
^{b}Responses and score following the 4-week single blind placebo run-in were utilized to establish baseline

### Additional Notes for Table 1B

### Primary Endpoints

SFQ28 (AS): Change from baseline to end-of-study in the Arousal-Sensation (AS) domain score of the SFQ28
FSDS-DAO (Q14): Change from baseline to end-of-study in score for feeling concerned by difficulties with sexual arousal (Item 14 of the FSDS-DAO)

### Secondary Endpoint

Arousal Diary (SSE): Change from baseline to end-of-study in number of Satisfactory Sexual Events (SSEs)

### Exploratory Endpoints

Arousal Diary (AS): Change from baseline to Week 4 (Visit 5), Week 8 (Visit 6) and Week 12 (Visit 7) of the Double-Blind Dosing Period.
Arousal Diary (AL): Change from baseline to Week 4 (Visit 5), Week 8 (Visit 6) and Week 12 (Visit 7) of the Double-Blind Dosing Period.
Arousal Diary (GA): Change from baseline to Week 4 (Visit 5), Week 8 (Visit 6) and Week 12 (Visit 7) of the Double-Blind Dosing Period.
Arousal Diary (SSE): In addition to the secondary endpoint analyses described above, change from baseline to Week 4 (Visit 5) and Week 8 (Visit 6) of the Double-Blind Dosing Period.
SFQ28 (AL): Change from baseline to Week 4 (Visit 5), Week 8 (Visit 6) and Week 12 (Visit 7) of the Double-Blind Dosing Period.
SFQ28 (AC): Change from baseline to Week 4 (Visit 5), Week 8 (Visit 6) and Week 12 (Visit 7) of the Double-Blind Dosing Period.
SFQ28 (Desire): Change from baseline to Week 4 (Visit 5), Week 8 (Visit 6) and Week 12 (Visit 7) of the Double-Blind Dosing Period.
SFQ28 (Orgasm): Change from baseline to Week 4 (Visit 5), Week 8 (Visit 6) and Week 12 (Visit 7) of the Double-Blind Dosing Period.
SFQ28 (AS): In addition to the primary endpoint analyses described above, change from baseline to Week 4 (Visit 5) and Week 8 (Visit 6) of the Double-Blind Dosing Period.
FSDS-DAO: Change from baseline in the FSDS-DAO total score from Week 4 (Visit 5), Week 8 (Visit 6) and Week 12 (Visit 7) of the Double-Blind Dosing Period.
FSDS-DAO (Q14): In addition to the primary endpoint analyses described above, change from baseline in the FSDS-DAO Item 14 from Week 4 (Visit 5) and Week 8 (Visit 6) of the Double-Blind Dosing Period.
PGI-S: Change from baseline to Week 4 (Visit 5), Week 8 (Visit 6) and Week 12 (Visit 7) of the Double-Blind Dosing Period.
PGI-C: Change from baseline to Week 4 (Visit 5), Week 8 (Visit 6) and Week 12 (Visit 7) of the Double-Blind Dosing Period.
PGI-F: Change from baseline to Week 4 (Visit 5), Week 8 (Visit 6) and Week 12 (Visit 7) of the Double-Blind Dosing Period.
PGI-C Concern: Change from baseline to Week 4 (Visit 5), Week 8 (Visit 6) and Week 12 (Visit 7) of the Double-Blind Dosing Period.
PGI-C SSE: Change from baseline to Week 4 (Visit 5), Week 8 (Visit 6) and Week 12 (Visit 7) of the Double-Blind Dosing Period.
PBE: Patient Benefit Evaluation

TABLE 2
Schedule of Events for Safety Evaluations

| | | Assessments | | | | | | | |
|---|---|---|---|---|---|---|---|---|---|
| | | | | Start of | | | | | |
| | | | Start of | Single- | | | | | |
| | | | No | Blind | Start of Each Month of | | | | |
| | | Medical | Drug | Placebo | Double-Blind Dosing | | | | Follow-Up |
| | | Screening | Run-In | Run-In | Period | | | | Assessment |
| | | Visit | | | | | | | |
| | | 1 | 2 | 3 | 4 | 5 | 6 | 7 | Phone Call |
| | | Study Weeks | | | | | | | |
| | | 0 | 1 | 5 | 9 | 13 | 17 | 21 | 22 |
| | | Study Days | | | | | | | |
| | | | | Visit | Visit | Visit | Visit | Visit | |
| | | | | 2 + | 3 + | 4 + | 5 + | 6 + | |
| Assessment | | Day −28 | | 28-31 | 28-31 | 28-31 | 28-31 | 28-31 | Visit 7 + |
| Category | | to −1 | 0 | days | days | days | days | days | 4-10 days |
| Informed | Informed Consent | X | | | | | | | |
| Consentª | Inclusion/Exclusion | Xª | Xª | X | X | X | X | X | |
| | Medical History | X^{a} | | | | | | | |
| Eligibility | Clinical Interview | X | | | | | | | |
| Determination | PHQ-8 | X | X | X | X | | | | |
| | GAD-7 | X | X | X | X | | | | |
| Study | Randomization | | | | X | | | | |
| Administration | Investigational | | | X^{h} | X | X | X | | |
| | Product Dispensed | | | | | | | | |
| | Investigational | | | | X | X | X | X | |
| | Product Returned | | | | | | | | |
| | to Site | | | | | | | | |
| Safety | Vulvar-Vaginal | X | X | X | X | X | X | | |
| Assessments | Exam^{b} | | | | | | | | |
| | Physical/GYN | X | | | | | | | |
| | Exam | | | | | | | | |
| | Lab Assessments^{c} | X | | | | | | | |
| | Serum Pregnancy | X | | | | | | | |
| | Test | | | | | | | | |
| | Pap Smear^{d} | X | | | | | | | |
| | Urine Pregnancy | —^{e} | X | X | X | X | X | X^{e} | |
| | Test | | | | | | | | |
| | Electrocardiogram^{f} | X | | | | | | | |
| | Vital Signs | Xª | X^{a} | X | X | X | X | X | |
| | Concomitant | X | X | X | X | X | X | X | |
| | Medication | | | | | | | | |
| | Reviewª | | | | | | | | |
| | Adverse Events | | X^{g} | X | X | X | X | X | X |
| | Participant | | | | | | | | |
| | Adverse Events | | X^{g} | X | X | X | X | | X |
| | Sexual Partner | | | | | | | | |

^{a}For both Patient and her Partner (all partners are required to attend Visits 1 and 2; same sex partners of childbearing potential will also attend Visit 7). Partners are permitted to perform vital signs utilizing a study-allocated blood pressure cuff from home. If self-assessment is chosen, partners will be seen remotely through a telemedicine visit.
^{b}The vulvar-vaginal examination will be performed using vulvoscopy (using a colposcope) to determine the level of irritation at every visit.
^{c}Laboratory assessments (including chemistry, hematology, thyroid stimulating hormone (TSH), follicle stimulating hormone (FSH), albumin, sex hormone binding globulin (SHBG), total testosterone, estradiol, prothrombin time, urinalysis), urine drug screen, serum pregnancy test, serology (including HIV antibodies), Nucleic Acid Amplification Test (NAAT) for sexually transmitted infections (gonorrhea, chlamydia, trichomoniasis), bacterial vaginosis and yeast will be performed following successful completion of Visit 1.
^{d}Pap smear will be performed unless the patient has had pap smear within 3 years of Visit 1 and is able to provide documentation.
^{e}A Urine pregnancy test will be performed on the patient's partner at the screening visit and at Visit 7 (or early termination), if she is a woman of childbearing potential.
^{f}A single, standard 12-lead ECG will be collected at Visit 1 for medical screening purposes.
^{g}Adverse events will be initially captured for the patient using an eDiary following visit 2 and assessed by the PI or designee. Any adverse events reported by the partner will be captured in the source document and in the eCRF by study site staff.
^{h}All patients are dispensed Placebo Cream at Visit 3.

### Investigational Product (IP), Dosage, and Mode of Administration:

Collectively, the Single Blind IP and the two Double-Blind IPs are referred to as the Investigational Product (IP) throughout the protocol. Single Blind IP was the Placebo Cream. Double-Blind IP was either Sildenafil Cream, 3.6% or Placebo Cream. The IP was provided in 30-gram tubes. Patients were randomized for entry into the Double-Blind Dosing Period in a 1:1 ratio via Interactive Response Technology (IRT) and provided with the corresponding tube. There were no allocation errors. Dosing cards were supplied to ensure the accurate measurement of the dose administered. The patient was instructed that approximately 50% of the IP is to be applied externally to the vulva (i.e., clitoris, vestibule, labia minora) and approximately 50% is to be applied intravaginally. The patients were instructed to use the dosing cards to measure out the appropriate amount of cream for the intravaginal and vulvar application. The patient was told to gently apply the cream, internally and externally, as instructed.

### Study Design and Methodology:

Described herein is a Phase 2b, multi-center, multiple-dose, double-blind, placebo-controlled study to evaluate the efficacy and safety of Sildenafil Cream, 3.6% in premenopausal patients with FSAD.

The study consisted of four periods—the Medical Screening Period, the No-Drug Run-In Period, the Single-Blind Placebo Run-In Period, and the Double-Blind Dosing Period. All patients were also contacted following completion of their final clinic visit for a Safety Follow-up Period (Phone Call). Table 2 provides an overview of the schedule of events and visit requirements for the study and instruments and assessments utilized-(i) diagnosis, (ii) inclusion and exclusion criteria, and (iii) primary, secondary and exploratory endpoints.

### Medical Screening Period: (Visit 1)

Following completion of a telephone screen (<28 days prior to Visit 1) or in person discussion, eligible participants were scheduled for Visit 1 and completed the following five steps to confirm eligibility to advance to the No Drug Run-Period (Visit 2). Table 1B provides a complete list of required assessments.

### Step 1—FSAD Screening Tools for Inclusion and to Establish Baseline Sexual Function

After patients provided written informed consent, FSAD symptoms-including decreased genital sensitivity and distress related to sexual problems- and baseline level of sexual functioning were assessed by the following two Patient Reported Outcome (PRO) instruments:
1. Female Sexual Distress Scale-Desire, Arousal, Orgasm (FSDS-DAO)
   a. total score>18 to confirm distress related to sexual dysfunction
2. Sexual Function Questionnaire-SFQ28
   Step 2—Additional Inclusion Criteria-Patients May be Entered into the Study if they Meet all of the Following Criteria:
1. Patient must be a premenopausal woman, 21 years or older.
2. Patient is fluent in the English language.
3 Patient is capable of understanding and complying with the protocol and agrees to sign the informed consent document.
4. Patient has been in a stable, monogamous, relationship that is secure and communicative, for at least 6 months prior to Visit 1. The relationship is with one sexual partner, who is sexually functional, both psychologically and physically. The partner will be consistent and available throughout the duration of the study.
5. Based on the clinical interview, in their past, patient had experienced ‘normal’ sexual functioning for at least 2 years or longer. Patient has had sexual activity at least two times each month during the last 6 months and agrees to have sexual activity at least two times each month for the duration of the study. Sexual activity can include any activity which may result in sexual stimulation or sexual pleasure e.g., intercourse, caressing, foreplay, masturbation, and oral sex.
   a. Patients who have experienced a recent major life stress (e.g., loss of income, death of a family member) or relationship discord that could interfere with sexual activity (except distress related to FSAD) will be excluded.
6. Women of childbearing potential must agree to continue using an acceptable form of birth control during the study and be on a stable dose or have had the insert/implant for at least 6 months without complication prior to Visit 1 and agree to continue to stay on their dose of birth control throughout the duration of the study.
   a. Acceptable forms of birth control include the following: intrauterine system [IUS], progestin and/or estrogen-containing hormonal oral contraceptives, contraceptive patch, contraceptive implant, contraceptive injection, or the copper-containing intrauterine device (IUD).
   b. Vaginal forms of contraception such as contraceptive foams/gels, diaphragms, penile or vaginal condoms, contraceptive vaginal rings, and sponges are not considered acceptable methods of birth control in this study.
   c. While latex and polyisoprene condoms are not an acceptable form of contraception, they may be used for the prevention of sexually transmitted infections.
7. Patient has a body mass index (BMI) from 18 to 35 kg/m², inclusive.
8. Patient has had a Pap smear performed within three years prior to Visit 1 and can provide documentation indicating normal test results (based on current guidelines as published by the U.S. Preventive Services Task Force).
   a. If the patient cannot provide documentation, a Pap smear will be performed at Visit 1. Patients with abnormal findings will be excluded from study participation and be referred for follow-up medical care as appropriate.
9. Patient is medically healthy with no clinically significant medical history, physical examination, gynecological history and examination, laboratory profiles (e.g., hematology, urinalysis), vital signs (e.g., uncontrolled hypertension), or ECG.
   a. Patients who have clinically significant ECG abnormalities at Visit 1 will be excluded.
   b. Patients with controlled, treated hypertension (i.e., <140/90 mmHg, using no more than two antihypertensive medications excluding alpha blockers and nitrates for the past 6 months on a stable dose) will be considered eligible.
   c. Patients with controlled, treated thyroid disease on a stable medication and dose for the past 6 months will be considered eligible. TSH must be within normal range (confirmed by laboratory test).
10. The participant must agree to not use vaginal hormone therapy (e.g., vaginal estrogen, intravaginal prasterone), vaginal or vulvar lubricants, spermicides, creams or gels, contraceptive foams or vaginal douche products throughout the study period.
   a. Women who self-report abnormal physiological vaginal discharge will be excluded until abnormal physiological discharge symptoms resolve.
   b. Patients using systemic (transdermal or oral) therapy must be on a stable dose for at least 6 months prior to Visit 1.

### Step 3—Exclusion Criteria-Patients Meeting any of the Following Criteria Will not be Entered in the Study:

1. Patient is nursing or pregnant (based on positive serum pregnancy test), wishes to become pregnant or begin nursing during the study period or was pregnant or nursing within 6 months prior to Visit 1.
2. Patient has more than one sexual partner.
3. Patient is postmenopausal (surgically induced or natural) meeting any of the following criteria:
   a. Bilateral oophorectomy, with or without hysterectomy;
   b. 12 months of spontaneous amenorrhea;
   c. 6 months of spontaneous amenorrhea with serum FSH levels>40 mIU/mL (except for those women on hormone replacement therapy).
4. Patient has any disorder or a history of any disorder that may prevent the successful completion of the study.
5 Patient has a significant cardiovascular, hepatic, metabolic, renal, respiratory, gastrointestinal, endocrine, immunologic, dermatologic, hematologic, neurologic, genitourinary or other unstable medical condition that would contraindicate administration of study medication, interfere with study evaluation, limit study participation, or confound the interpretation of study results.
6. Patient has a history of unresolved sexual trauma or abuse that is contributing to any sexual dysfunction problem (desire, arousal, orgasm, etc.) as determined by the clinical interview.
7. Patient with any history of active peptic ulcers or clinically significant bleeding disorders.
8. Patient with a history of clitoral priapism or conditions which may predispose them to clitoral priapism (such as sickle cell anemia, multiple myeloma, or leukemia).
9. Patients with a history of myocardial infarction, stroke, or life-threatening arrhythmia within 6 months prior to Visit 1; patients with resting hypotension (BP<90/50 mmHg); or any history of coronary disease causing angina; or congestive heart failure requiring medical intervention; patients with underlying conditions which can be particularly sensitive to the actions of vasodilators including patients with left ventricular outflow obstruction (e.g., aortic stenosis, idiopathic hypertrophic subaortic stenosis) and patients with impaired autonomic control of blood pressure or for those for which sexual activity is inadvisable due to their underlying cardiovascular status.
10. Patient with a history of hearing loss.
11. Patient has retinitis pigmentosa, even if the patient feels clinically well at the time of Visit 1. Patients with retinitis pigmentosa will be identified by specifically asking whether they have the condition, if there are visual signs and symptoms of the condition (including questioning patients as to whether they have difficulty seeing at night or in low light, and if they have any visual field deficits that indicate a loss of peripheral or central vision), or if there is a family history.
12. Patient has a history of orthostatic hypotension or orthostatic hypotension which is present at Visit 1, defined as a drop in systolic blood pressure≥20 mm Hg, a drop in diastolic blood pressure≥10 mm Hg, an increase in pulse by 20 beats per minute or experiencing lightheadedness or dizziness at 1 or 3 minutes after the change in position from supine to standing.
13. Patient has a primary complaint of anorgasmia, vaginismus, low desire, or any other primary sexual complaint aside from problems with genital arousal, as determined by the clinical interview.
14. Patient has untreated dyspareunia, vulvovaginal infection or inflammation, inflammatory disorders of the vulva or vagina, vestibulodynia, clitorodynia, symptomatic vulvovaginal atrophy (defined as women having≤5% of superficial cells on vaginal smear at baseline, vaginal pH above 5, patient-identified moderate or severe symptom(s) of vulvovaginal atrophy).
15. Patient has undergone major pelvic surgery or abdominal surgery that may have caused nerve damage, including, vulvectomy, colostomy, cystostomy, hysterectomy and bladder neck suspension.
16. Patients with comorbid conditions that may cause underlying neurological impairment (i.e., Type 1 or Type 2 diabetes, metabolic syndrome, stroke, myasthenia gravis, multiple sclerosis or spinal cord damage).
a. Patients who are symptomatic of peripheral neuropathy will be excluded.
17. Patients with pelvic nerve damage secondary to trauma will be excluded.
18. With the exception of anxiety and depression, patient has any current and/or previously reported diagnoses of DSM-IV-TR axis I disorders (e.g., schizophrenia, bipolar disorder) including delirium, dementia and amnestic disorders.
   a. Patients diagnosed with anxiety or depression must be controlled, as determined by the Investigator, and if on a medication (i.e. SSRIs, SNRIs, buspirone, bupropion and benzodiazepines), on a stable dose for at least the past 6 months.
   b. Patients must have a total score<10 indicating mild depression on the Patient Health Questionnaire-8 (PHQ-8, Appendix G) (a screening and assessment instrument for depression).
   c. Patients must have a total score<6 indicating mild anxiety on the Generalized Anxiety Disorder (GAD-7, Appendix H) (a screening and assessment instrument for anxiety).
   d. Patients who have any history of antipsychotic therapy within the last year will be excluded.
19. Patients that have a history of gynecological cancer or are under active treatment (or recently completed treatment within the last 6 months) for any cancer which would interfere with the patient's ability to successfully complete the study will be excluded.
   a. Patients who have been previously treated for dysplasia (precancerous changes) may be included as long as the patient received localized treatment (e.g., cryosurgery or laser).
   b. Patients with a history of radiation to the pelvis will be excluded.
20 Patient has any surgical or medical condition that may interfere with the absorption, distribution, metabolism, or excretion of the test article.
21. Patient self-reports a history of substance abuse in the two years prior to Visit 1 or exhibits signs of current substance abuse.
22. Patient self-reports a history of alcohol abuse in the two years prior to Visit 1 or exhibits signs of current alcohol abuse.
23. Patient has a history of non-arteritic ischemic optic neuropathy (NAION) or any underlying NAION risk factors.
24 Patient whose sexual function was affected (enhanced or worsened) by any medication within 28 days before Visit 1 and at any time prior to the No Drug Run-in Period of the study.
25 Patient is currently receiving treatment or has received treatment within 1 month (28 days) prior to Visit 1 of any of the following medications: guanylate cyclase stimulators (e.g., Riociguat), clonidine, strong CYP3A4 inhibitors, nitric oxide donors, such as organic nitrates or organic nitrites, and alpha blockers.
   a. Patients who are currently receiving treatment or have received treatment in the past 3 months for FSAD symptoms, pharmacologic (e.g., PDE5 inhibitors in any form except Sildenafil Cream, 3.6% or other experimental therapies used to enhance the arousal response) or non-pharmacologic treatment (e.g., sex therapy), will also be excluded.
26 Patient has positive findings from the urine drug screen (e.g., amphetamines, barbiturates, cocaine, methadone, and opiates) or alcohol breath test.
27. Patient has positive findings for sexually transmitted infection (trichomoniasis, gonorrhea, chlamydia), and human immunodeficiency virus (HIV) antibodies.
28 Patient reports having an outbreak (blisters, warts, or vesicles) due to any of the following sexually transmitted diseases: genital herpes or HPV at any time point in the past three months.
29 Patient has been diagnosed with chlamydia, trichomoniasis or gonorrhea in the past 3 months.
30 Patient has participated in any clinical research study evaluating another investigational drug or therapy within 30 days before Visit 1 (or 6 half-lives of the investigational agent, whichever is longer) and agrees not to participate in another clinical research study throughout the duration of the study.
31. Patient has any clinically significant abnormal findings on vulvar-vaginal examination performed during the physical and gynecological exams at Visit 1 (e.g., genital skin breaks, irritation, dermatoses, or lesions).
32. Patient currently has moderate to severe vaginitis, a vaginal infection including bacterial vaginosis, a yeast infection or the presence of yeast based on the Nucleic Acid Amplification Test (NAAT).
   a. If the patient has a vaginal infection or a positive test for yeast during the screening visit, they may be treated, and the screening visit rescheduled.
   b. If the patient develops a vaginal infection following enrollment into the study, study drug will be withheld, and the patient will be treated for the infection. If the infection cannot be resolved within two weeks the patient will be withdrawn from the study. Treatment of vaginal infections will be allowed twice following enrollment. If the patient develops a third vaginal infection they will be withdrawn from the study.
33. Patient has a pelvic or urinary tract infection.
34 Patient self-reports a known hypersensitivity or adverse reaction to any ingredients in the IP.

### Step 4—Laboratory Assessments

Blood, urine, and vaginal samples were taken for all patients who remain eligible for the study (successfully completing Steps 1-3 above). Patients who continue to meet the inclusion criteria following review of laboratory results were scheduled for the clinical diagnosis of FSAD.

### Step 5—Clinical Diagnosis of FSAD

Patients who met all the above inclusion and exclusion criteria underwent a formal clinical interview to diagnose primary FSAD which is acquired (i.e., not lifelong) and generalized (i.e., present regardless of contextual setting) and with symptoms which have been present for at least the past 6 months prior to Visit 1. Eligible patients must have a primary complaint of lack of genital responsiveness during sexual activity and regard their genital response as meaningful to their overall sexual experience. Eligible patients must also be experiencing distress because of their sexual arousal difficulties.

Patients who have secondary complaints related to low desire due to lack of genital arousal (Hypoactive Sexual Desire Disorder) will be considered eligible, as long as problems related to diminished genital arousal are deemed the most bothersome symptom.

Following completion of the Clinical Interview, FSAD diagnosis will be confirmed by the DSMIV-TR criteria. Eligible patients will be scheduled for Visit 2 within 14 days of Visit 1.

### No Drug Run—In Period-4 Weeks (Visit 2)

Following successful completion of all medical screening, including laboratory assessments, and confirmation of all inclusion and exclusion criteria, eligible patients and their partner were enrolled into a 4 week No Drug Run-In Period. Visit 2 is the first day of the 4 week No Drug Run-In Period. Patients will not be permitted to use any oral or topical products (e.g., Dream Cream, Zestra®, OTC lubricants, PDE-5 inhibitors, etc.) or undergo non-pharmacological intervention (i.e., sex therapy) for their arousal problems during this 4-week period. Both the patient and partner were provided with separate eDiary devices and relevant instructions for their use. Adverse events were recorded by both the patient and her partner using separate eDiary devices during the No Drug Run-In Period. Patients and their partner were instructed (by daily prompt) to complete the daily eDiary within 24 hours following the completion of each sexual activity during the 4-week No Drug Run-In Period. In addition, should a sexual event occur, the patient was prompted on the eDiary to confirm partnered sexual activity, and complete the Arousal Diary. Eligible patients were scheduled for Visit 3 in four weeks.

### Single-Blind Placebo Run-In Period—4 Weeks (Visit 3)

Following completion of the 4-week No Drug Run-In Period, eligible patients entered a 4-week Single-Blind Placebo Run-In Period. Visit 3 is the first day of the 4-week Single-Blind Placebo Run-In Period. At the initiation of the Single-Blind Placebo Run-In Period, inclusion and exclusion criteria for the study was confirmed and in addition, the patient's level of compliance completing the eDiary was evaluated.

Eligible patients completed the following instruments and assessments in the clinic at Visit 3 using the eDiary.
1. Sexual Function Questionnaire (SFQ28)
2. Female Sexual Distress Scale-Desire, Arousal, Orgasm (FSDS-DAO)
   a. total score must remain>18 to confirm distress related to sexual dysfunction
3. Patient Global Impression of Severity (PGI-S)
4. Patient Global Impression of Change (PGI-C)

Patients were provided with a single 30 g tube of Single Blind IP and instructed to apply the cream 10 to 20 minutes prior to each sexual activity. Patients and partners were also instructed to wash off any remaining treatment cream after all sexual activity has ended. Adverse events were recorded by both the patient and her partner using separate eDiary devices during the Single-Blind Placebo Run-In Period. Patients and their partner were instructed (by daily prompt) to complete the daily eDiary within 24 hours following the completion of each sexual activity during the 4-week No Drug Run-In Period.

In addition, should a sexual event occur, the patient was prompted on the eDiary to record the application of the IP, confirm partnered sexual activity, and complete the Arousal Diary. The eDiary responses, including the Arousal Diary, SFQ28, and the FSDS-DAO scores following the 4-week Single-Blind Placebo Run-In Period (Visit 4) were utilized to establish baseline. Eligible patients were scheduled for Visit 4 in four weeks.

### Double-Blind Dosing Period—12 Weeks (Visits 4-7)

Following completion of the 4-week Single Blind Placebo Run-In Period, eligible patients entered the Double-Blind Dosing Period. Visit 4 is the first day of the Double-Blind Dosing Period. At the initiation of the Double-Blind Dosing Period, inclusion and exclusion criteria was confirmed and in addition, the patient's level of compliance completing the eDiary and use of the investigational product was evaluated.

Eligible patients completed the following instruments and assessments in the clinic at Visit 4 using the eDiary.
1. Sexual Function Questionnaire (SFQ28)
2. Female Sexual Distress Scale-Desire, Arousal, Orgasm (FSDS-DAO)
   a. total score must remain>18 to confirm distress related to sexual dysfunction
3. Patient Global Impression of Severity (PGI-S)
4. Patient Global Impression of Change (PGI-C)

The eDiary responses, including the Arousal Diary, SQF28, and the FSDS-DAO scores following the 4-week Single Blind Placebo Run-In (Visit 4) were utilized to establish baseline.

During the Double-Blind Dosing Period, patients returned to the clinic every four weeks for efficacy and safety assessments. Visit 4 was the first day of the Double-Blind Dosing Period, with Visit 7 being the completion of this 12-week Double-Blind Dosing Period.

At Visits 4-6, patients were provided with a single 30 g tube of Double-Blind IP based on the randomization schedule. Patients returned the tube at subsequent visits (i.e., Visits 5-7) and a new tube was dispensed for the next month of double-blind treatment. Patients were instructed to apply the cream 10 to 20 minutes prior to each sexual activity. Patients and partners were also instructed to wash off any remaining treatment cream after all sexual activity has ended. Adverse events were recorded by both the patient and her partner using separate eDiary devices during the Double-Blind Treatment Period. Patients and their partner were instructed (by daily prompt) to complete the daily eDiary within 24 hours following the completion of each sexual activity during the 4-week No Drug Run-In Period. In addition, should a sexual event occur, the patient was prompted on the eDiary to record the application of the IP, confirm partnered sexual activity, and complete the Arousal Diary.

Patients completed the following instruments and assessments in the clinic at Visits 5-7 using the eDiary.
1. Sexual Function Questionnaire (SFQ28)
2. Female Sexual Distress Scale-Desire, Arousal, Orgasm (FSDS-DAO)
3. Patient Global Impression of Severity (PGI-S)
4. Patient Global Impression of Change (PGI-C)

Patients completed the Patient Benefit Evaluation (PBE) in the clinic at Visit 7. Adverse events were recorded by both the patient and her partner using separate eDiary devices during the Double-Blind Treatment Period. Patients and their partner were instructed (by daily prompt) to complete the daily eDiary within 24 hours following the completion of each sexual activity during the 4-week No Drug Run-In Period. In addition, should a sexual event occur, the patient was prompted on the eDiary to record the application of the IP, confirm partnered sexual activity, and complete the Arousal Diary.

A semi-structured Exit Interview was conducted over the telephone to a select number of patients at the end of the study (Visit 7) or following study withdrawal to provide a more in-depth, qualitative description of the patient's symptoms, response to treatment, what constitutes a meaningful change on both the PGI-S and PGI-C and why, and treatment satisfaction to augment the quantitative assessments captured with the PRO instruments.

### Safety Assessment Follow Up (Phone Call):

Each patient and her partner were contacted by telephone 7±3 days following the last visit in the Double-Blind Dosing Period to document any changes in concomitant medications and potential adverse events that may have occurred following the final study visit (Visit 7). The maximum study duration is approximately six (6) months.

### Evaluation of Recall Period

At the end of the single blind placebo run-in and at weeks 4, 8 and 12 of the double-blind treatment period, the correlation between the 24-hour recall period and the 4-week recall period were evaluated for all patients who complete both the Arousal Diary and the SFQ28. In addition, at the same intervals a sub-set of patients (n=30), selected randomly via Interactive Response Technology (IRT), who complete the SFQ28 but do not complete the Arousal Diary, were evaluated to investigate whether completion of the diary questions influences patient SFQ28 Arousal (Sensation) domain scores.

### Patient Withdrawal

For patients who have signed the Informed Consent Form at Visit 1 and those who drop out of the study early who have received at least one dose of IP were instructed to complete the relevant PRO instruments (SFQ28, FSDS-DAO, etc.). These patients were also tabulated by reason for discontinuation in the final study tables, listings, and figures.

### Withdrawal Criteria

Patients may choose to withdraw from the study at any time for any reason. In addition, patients may be withdrawn from the study for any of the following reasons:
The patient or her partner is unwilling or unable to adhere to the protocol,
During the course of the study, the patient develops symptoms or conditions listed in the exclusion criteria.
During the course of the study the patient develops any of the following conditions: visual adverse effect, severe skin reaction, symptomatic orthostatic tachycardia, symptomatic hypotension, asymptomatic hypotension, or asymptomatic orthostatic hypotension,
Any SAE, clinically significant AE, severe laboratory abnormality, intercurrent illness, or other medical condition that indicates to the Investigator that continued participation is not in the best interest of the patient, or
Other medical reason

### Stopping Criteria

The trial will be halted if >1% of patients or partners treated with active cream report significant post-dose symptoms associated with hypotension (dizziness, lightheadedness, nausea) which results in fainting (syncope) and significant injury.

### Inclusion Criteria—For Patient's Partner Eligibility

Patient's partner may be entered into the study if they meet all the following criteria at Visit 1:
1. Partner must be age 21 or older.
2. Partner must be fluent in the English language.
3. Partner is capable of understanding and complying with the protocol and agrees to sign the informed consent document.
4. Partner has been in a stable, monogamous, relationship with the patient that is secure and communicative, for at least 6 months prior to Visit 1. The partner is sexually functional, both psychologically and physically. The partner will be consistent and available throughout the duration of the study.

### Exclusion Criteria—For Patient's Partner Eligibility

Partners meeting any of the following criteria at Visit 1 will not be entered in the study:
1. Partner is nursing or pregnant (based on a positive urine pregnancy test) or wishes to become pregnant during the study period.
2. Partner has any disorder or a history of any disorder that may prevent the successful completion of the study.
3. Partner has had treatment currently or within 1 month (28 days) prior to Visit 1 of any of the following will be excluded: intravaginal hormone products (e.g., estrogen or prasterone), guanylate cyclase stimulators (e.g., Riociguat), phosphodiesterase (PDE) type 5 inhibitors, nitric oxide donors such as organic nitrates or organic nitrites.
4. Partner has a significant cardiovascular, hepatic, metabolic, renal, respiratory, gastrointestinal, endocrine, immunologic, dermatologic, hematologic, neurologic, genitourinary, or psychiatric disease or other unstable medical condition or any concomitant medications that would contraindicate administration of study medication, interfere with study evaluation, limit study participation, or confound the interpretation of study results.
5. Partner with a history of myocardial infarction, stroke, or life-threatening arrhythmia within 6 months prior to Visit 1; patients with resting hypotension (BP<90/50 mmHg); or any history of coronary disease causing angina; or congestive heart failure requiring medical intervention; partners with underlying conditions which can be particularly sensitive to the actions of vasodilators including patients with left ventricular outflow obstruction (e.g., aortic stenosis, idiopathic hypertrophic subaortic stenosis) and partners with impaired autonomic control of blood pressure or for those for which sexual activity is inadvisable due to their underlying cardiovascular status.
6. Partner has a history of orthostatic hypotension or orthostatic hypotension which is present at Visit 1, defined as a drop in systolic blood pressure≥20 mm Hg, a drop in diastolic blood pressure≥10 mm Hg, an increase in pulse by 20 beats per minute or experiencing lightheadedness or dizziness at 1 or 3 minutes after the change in position from supine to standing.
7. Partner has a history of priapism or conditions which may predispose him to priapism (such as sickle cell anemia, multiple myeloma, or leukemia).
8. Partner has a current pelvic or urinary tract infection.
9. Partner self-reports having any sexually transmitted infection (including gonorrhea, trichomoniasis, and chlamydia) in the past 3 months.
10. Partner self-reports a diagnosis of human immunodeficiency virus (HIV).
11. Partner self-reports having an outbreak (blisters, warts, or vesicles) due to any of the following sexually transmitted diseases: genital herpes or HPV at any time point in the past three months.
12. Partner has a history of non-arteritic ischemic optic neuropathy (NAION) or any underlying NAION risk factors.
13. Partner has participated in any clinical research study evaluating another investigational drug or therapy within 30 days before Visit 1 (or 6 half-lives of the investigational agent, whichever is longer). Partner must also agree not to enter into any other clinical trial for the duration of the study.
14. Partner reports a known hypersensitivity or adverse reaction to any ingredients in the Investigational Product (IP).

### Investigational Product (IP)

Collectively, the Single-Blind IP and the Double-Blind IP is referred to as the Investigational Product (IP) throughout this protocol. The Single-Blind IP will be a Placebo Cream and the Double-Blind IP will be either Placebo Cream or Sildenafil Cream, 3.6%, depending on the randomization scheme. The Single-Blind IP and Double-Blind IP will be provided in 30 gram, white, aluminum tubes. A single 30-gram tube will allow for nine (9) 2-gram applications. Dosing Cards to be used by the patient to ensure accuracy of dosing will also be provided. Cream was water-based, white to off-white in appearance, and identical in color, smell, and consistency between Sildenafil Cream and Placebo Cream.

Sildenafil Cream, 3.6% is a white to off-white cream containing 5% (w/w) sildenafil citrate and salts and excipients as listed in Table 3 resulting in a 3.6% Sildenafil concentration. Packaging and labeling of IP adhered to all applicable regulatory requirements. IP was stored at room temperature (20-25° C.) in a secure, temperature and humidity monitored area.

TABLE 3
Formula for Sildenafil Cream, 3.6%

| Ingredient | % w/w |
|---|---|
| Purified water USP | 40.75 |
| Oleic acid NF | 1.00 |
| Polysorbate 20 NF | 2.00 |
| Squalane NF | 4.00 |
| Isopropyl myristate NF | 1.00 |
| Cetyl alcohol NF | 7.00 |
| Glyceryl stearate SE | 7.00 |
| Sildenafil citrate USP | 5.00 |
| Propylene glycol USP | 8.50 |
| Xanthan gum NF | 0.80 |
| Potassium chloride USP | 5.00 |
| L-Arginine•HCl USP | 7.50 |
| Trisodium citrate dihydrate USP | 10.00 |
| Sodium benzoate USP | 0.20 |
| Gluconolactone USP | 0.25 |

USP = United States Pharmacopeia/NF = National Formulary/SE = Self-Emulsifying

The Placebo Cream will include the same ingredients as in the Sildenafil Cream, 3.6% but without the active ingredient, sildenafil citrate. It will be matched in appearance, smell, consistency, and color to Sildenafil Cream, 3.6%. The complete list of ingredients is listed below in Table 4.

TABLE 4
Formula for Placebo Cream

| Ingredient | % w/w |
|---|---|
| Purified water USP | 42.99 |
| Oleic acid NF | 1.05 |
| Polysorbate 20 NF | 2.10 |
| Squalane NF | 4.20 |
| Isopropyl myristate NF | 1.05 |
| Cetyl alcohol NF | 7.36 |
| Glyceryl stearate SE | 7.36 |
| Sildenafil citrate USP | 0.00 |
| Propylene glycol USP | 8.95 |
| Xanthan gum NF | 0.84 |
| Potassium chloride USP | 5.26 |
| L-Arginine•HCl USP | 7.88 |
| Trisodium citrate dihydrate USP | 10.51 |
| Sodium benzoate USP | 0.20 |
| Gluconolactone USP | 0.25 |

USP = United States Pharmacopeia/NF = National Formulary/SE = Self-Emulsifying

### Single Blind Placebo Run-in Period

One 30-gram tube of Placebo Cream for the Single-Blind Run-In Period and will be dispensed to the patient at Visit 3 allowing for nine (9) 2-gram applications of cream. Patients will be instructed to apply 2 grams of cream 10 to 20 minutes prior to each sexual activity. Specific dosing instructions will be provided separately to the patient.

### Double-Blind Dosing Period

Each month, IP was dispensed in a 30-gram tube, along with nine, 2-gram dosing cards. Participants were instructed to apply no more than one 2-gram application of product per 24 hours and no more than 9 applications per month. After dispensing a 2-gram portion on to the dosing card, participants used their index finger to apply 2 grams of the IP approximately 10-20 minutes prior to the sexual event. One gram was applied externally to the anterior labial commissure, prepuce of the clitoris, glans, frenulum, vestibule, and labia minora. The other one gram was applied to the anterior distal vagina to an approximate depth 0-3 cm or about halfway between the distal and proximal interphalangeal joints, targeting the distal, lower ⅓ of the vagina. Participants were instructed to wash off the cream and have their partner wash off the cream, if relevant, after the sexual experience. Use of over 9 applications per month was recorded as a protocol violation.

### IP Dosing Application Procedures

The Dosing Card must be used to measure the correct amount of cream to be applied based on the instructions below for both the Single-Blind Placebo Run-In Period and Double-Blind Dosing Period. Patients were instructed to measure the correct amount of cream as follows: “To measure the right amount of cream, place the dosing card on a flat surface. Squeeze cream onto dosing card by evenly filling in the rectangle outlined on the card. Make sure the cream covers the entire area in the rectangle outlined on the dosing card.”

The rectangle on the dosing card (sized to deliver 2 grams of cream) was divided into two equal sections to be used to apply the cream externally to the pre-specified vulvar area and internally intravaginally, respectively.

Approximately 50% of the cream was to be applied externally to the vulva (i.e., clitoris, vestibule, labia minora) and approximately 50% was to be applied intravaginally. The patients were instructed to indicate time of application on the eDiary and use the Dosing Card to administer the appropriate amount of cream for the intravaginal and vulvar application as follows:

### External Cream Application:

The patient was told to apply approximately half of the cream from the Dosing Card externally with the index finger, as described below:

Step 1. Beginning at the anterior labial commissure, gently apply the cream to the prepuce of the clitoris, glans, and frenulum.

Step 2. Continue gently applying the cream to the vestibule.

Step 3. Continue gently applying the cream to the labia minora.

(NOTE: avoid applying to the labia majora and avoid application of product to pubic hair)

### Internal Cream Application:

Step 4. With index finger, obtain the other half of the cream and gently apply intravaginally to the anterior distal vagina in a sweeping motion (approximate depth 0-3 cm or about halfway between the distal and proximal interphalangeal joints, targeting the distal, lower ⅓ of the vagina).

Extensive site training for patients in advance of the Single-Blind Placebo Run-In Period was performed to ensure patients understand the instructions for use of the product. The patient was instructed to wash their hands and avoid wiping or washing off any of the area where cream has been applied. Patients and partners were also instructed to wash off any remaining treatment cream after all sexual activity has ended.

### IP Dosing Frequency

The IP was dispensed to allow for a total of nine (9) 2-gram applications of cream during each monthly interval (nine, 2-gram doses can be reliably delivered from the 30-gram tube). The patient was instructed to apply the cream 10 to 20 minutes prior to each sexual activity but not to exceed a maximum of nine (9) doses during each 4-week period. The patient was further instructed not to apply the cream within 24 hours of a prior application.

### Dosing Schedule

### Single-Blind Placebo Run-In Period (Visit 3)

Following completion of the 4-week No Drug Run-In Period, eligible patients entered a 4-week Single Blind Placebo Run-In Period. During this 4-week period of the study, patients received Single Blind IP provided in a single 30-gram tube dispensed to the patient in a single-blind manner, providing for the monthly supply. Patients were instructed to apply 2 grams of cream 10 to 20 minutes prior to each sexual activity.

### Double Blind Dosing Period (Visits 4-7)

Following completion of the 4-week Single-Blind Placebo Run-In Period, eligible patients entered a 12-week Double Blind Dosing Period. During this 12-week period of the study, based on the randomization schedule, patients received the same Double-Blind IP at each visit, provided in 30-gram tubes. A single 30-gram tube was dispensed to the patient at Visit 4, Visit 5, and Visit 6 providing for a monthly supply. Patients were instructed to apply 2 grams of cream 10 to 20 minutes prior to each sexual activity.

### Prior and Concomitant Medications

### Allowed Concomitant Medications

Patients and their respective partner were allowed to take concomitant medication as needed with guidance from the Medical Monitor, with the exception of medications listed below.

All concomitant medications were recorded for patients and their respective partner enrolled in the study from 28 days prior to Visit 1 up until the completion of Visit 7.

Changes in concomitant medications were captured in the eDiary for patients and their partner.

### Prohibited Concomitant Medications—Patient and Her Partner

Patients who have positive findings from the urine drug screen (e.g., amphetamines, barbiturates, cocaine, methadone, and opiates) or alcohol breath test will be excluded from further study participation. Patients will be excluded from participating in the study if they are currently taking or have had treatment within the last 28 days prior to Visit 1 with any of the following medications: guanylate cyclase stimulators (e.g., Riociguat), clonidine, strong CYP3A4 inhibitors, nitric oxide donors, such as organic nitrates or organic nitrites, or alpha blockers. Patients who are currently receiving treatment or have received treatment in the past 3 months for FSAD symptoms, pharmacologic (e.g., PDE5 inhibitors in any form except Sildenafil Cream, 3.6% or other experimental therapies used to enhance the arousal response) or non-pharmacologic treatment (i.e., sex therapy), will also be excluded.

Patients are prohibited from using vaginal hormonal products (e.g., vaginal estrogen intravaginal prasterone), vaginal or vulvar lubricants, spermicides, creams or gels, contraceptive foams or vaginal douche products throughout the study period. Partners who are currently taking or have taken within 1 month (28 days) prior to Visit 1 guanylate cyclase stimulators (e.g., Riociguat), clonidine, strong CYP3A4 inhibitors, phosphodiesterase (PDE) type 5 inhibitors, nitric oxide donors, such as organic nitrates or organic nitrites, will be excluded. Patients who are using contraceptive vaginal sponges will be excluded. The patient or her partner will be excluded from participating in the study if she is currently taking or has had treatment with and prohibited medications as outlined in the respective inclusion and exclusion criteria for the study.

### Safety Assessments for Patients and Partners

### Adverse Events (AEs)

Adverse events were collected for patients and their respective partner at each visit starting from signing informed consent. AEs reported prior to the first application of IP were recorded on the AE eCRF but not be included in the summaries of treatment-emergent AEs (TEAEs). AEs reported after the first application of IP were recorded on the AE eCRF. AEs occurring since completing their last clinic visit were recorded 7±3 days after the conclusion of the Double-Blind Dosing Period (Visit 7).

### Reporting of Adverse Events

The patient and her partner were instructed to immediately notify of any serious medical issues that occur during the course of the study. The patient and their partner were instructed to call 911 in the event of a life-threatening emergency. The patient recorded all AEs in the eDiary, per the instructions provided. The patient completed the eDiary within 24 hours of each sexual event and at any time during the course of the study at which time an AE may occur. The partner recorded all AEs in a separate eDiary, per the instructions provided. The partner completed the eDiary within 24 hours of each sexual event and at any time during the course of the study at which time an AE may occur. The eDiaries prompted both the patient and the partner every 24 hours to assess whether there has been a sexual event. If there was a sexual event, the eDiary provided the following prompt to the patient and the partner: ‘Since your sexual event, do you have any complaints?’ If the participant or partner answered “YES,” the eDiary would further prompt them for some of the more common side effects associated with oral sildenafil or genital creams, such as: (1) “Have you experienced any local irritation or discomfort?” (2) “Have you experienced any dizziness or lightheadedness?” and (3) “Are there any other complaints you would like to report?” The responses from participants who reported a complaint following sexual activity were sent to the site electronically for evaluation and triage within 24 hours. Additionally, participants that answered “YES” to complaints were reminded to contact their study site. Subjects were instructed to call the Investigator's emergency number if they did not receive a response within 24 hours of sending their complaint electronically. Participants were also instructed to report any complaints of local irritation or discomfort promptly to the investigator for follow-up.

If the participant confirmed via the eDiary that a sexual event had taken place with her partner, study site staff were prompted to contact the partner within 72 hours of the event to confirm whether or not the partner had any complaints and if so, the partner was further queried on common side effects associated with oral sildenafil (e.g., light headedness) or genital cream exposure (e.g., genital irritation). Partners were instructed to contact the Investigator if they did not receive a call within 72-hours of sexual activity with the participant.

Participants recorded TEAEs in the eDiary. Participants and sexual partners were invited to unscheduled visits if they had TEAE complaints that they wanted to have a physical exam to assess, or at the discretion of the investigator based on the AE reported.

Orthostatic vital signs were obtained on the participant at each visit, with heart rate and blood pressure measured in the supine position, and 1 and 3 minutes after standing. The same orthostatic vital signs were measured among participating sexual partners, either at the clinic or via telemedicine at each visit.

### Adverse Event

An adverse event is defined as any untoward medical occurrence associated with the use of a drug in humans, whether or not considered drug related. An AE (also referred to as an adverse experience) can be any unfavorable and unintended sign (e.g., an abnormal laboratory finding), symptom, or disease temporally associated with the use of a drug and does not imply any judgment about causality. An AE can arise with any use of the drug (e.g., off-label use, use in combination with another drug) and with any route of administration, formulation, or dose, including an overdose.

### Suspected Adverse Reaction

A suspected adverse reaction is any AE for which there is a reasonable possibility that the drug caused the AE. For the purposes of Investigational New Drug (IND) safety reporting, “reasonable possibility” means there is evidence to suggest a causal relationship between the drug and the AE. Suspected adverse reaction implies a lesser degree of certainty about causality than adverse reaction, which means any AE caused by a drug.

### Life-Threatening AE or Life-Threatening Suspected Adverse Reaction

An AE or suspected adverse reaction is considered “life-threatening” if its occurrence places the patient or her partner at immediate risk of death. It does not include an AE or suspected adverse reaction that, had it occurred in a more severe form, might have caused death.

### Serious AE or Serious Suspected Adverse Reaction

An AE or suspected adverse reaction is considered “serious” if it results in any of the following outcomes:
Death,
A life-threatening AE-see definition above,
inpatient hospitalization or prolongation of existing hospitalization,
A persistent or significant incapacity or substantial disruption of the ability to conduct normal life functions, and
A congenital anomaly/birth defect.

Important medical events that may not result in death, be life-threatening, or require hospitalization may be considered serious when, based upon appropriate medical judgment, they may jeopardize the patient or her partner and may require medical or surgical intervention to prevent one of the outcomes listed in this definition. Examples of such medical events include allergic bronchospasm requiring intensive treatment in an emergency room or at home, blood dyscrasias or convulsions that do not result in inpatient hospitalization, or the development of drug dependency or drug abuse.

### Relationship to Investigational Drug

The assessment of causality must be provided for all AEs (serious and non-serious). The causality assessment is the determination of whether there exists a reasonable possibility that the investigational product (IP) caused or contributed to an AE.

None: No relationship between the experience and the administration of study drug; related to other etiologies such as concomitant medications or patient's clinical state.

Unlikely: The current state of knowledge indicates that a relationship is unlikely.

Possible: A reaction that follows a plausible temporal sequence from administration of the study drug and follows a known response pattern to the suspected study drug. The reaction might have been produced by the patient's clinical state or other modes of therapy administered to the patient, but this is not known for sure.

Probable: A reaction that follows a plausible temporal sequence from administration of the study drug and follows a known response pattern to the suspected study drug. The reaction cannot be reasonably explained by the known characteristics of the patient's clinical state or other modes of therapy administered to the patient.

Definite: A reaction that follows a plausible temporal sequence from administration of the study drug and follows a known response pattern to the suspected study drug and can be confirmed with a positive re-challenge test or supporting laboratory data.

### Severity

On the Adverse Event eCRFs, the terms: Mild, Moderate, Severe, or Life threatening will be used to describe the maximum intensity of the AE. For purposes of consistency, these intensity grades are defined as indicated below.

Mild: Awareness of sign or symptom, but easily tolerated

Moderate: Discomfort enough to cause interference with normal daily activities

Severe: Inability to perform normal daily activities

Life threatening: Immediate risk of death from the reaction as it occurred

### Screening Laboratory Assessments

The following laboratory assessments were performed following successful completion of Visit 1.
Serum chemistry: alanine aminotransferase, alkaline phosphatase, aspartate aminotransferase, bilirubin (total), blood urea nitrogen, creatinine, potassium, protein (total), sodium, chloride, and bicarbonate
Hematology: hematocrit, hemoglobin, platelets, red blood cell count, white blood cell count, and white blood cell (WBC) count differential only if the total WBC is abnormal.
Prothrombin time
TSH
FSH
Estradiol
Total Testosterone, SHBG, and Albumin
Urinalysis: bilirubin, blood, glucose, ketones, nitrites, pH, protein, specific gravity, urobilinogen, and leukocyte esterase.
HBsAg, HCV HIV and antibodies
NAAT (Nucleic Acid Amplification Test): bacterial vaginosis, yeast (Candida sp) and sexually transmitted infection (gonorrhea, chlamydia, trichomoniasis)
Urine drug screen for the following drugs of abuse: amphetamines, barbiturates, cocaine, methadone, and opiates.

Women were required to undergo a urine pregnancy test at every visit during the course of the study, with the exception of Visit 1 where a serum pregnancy test was performed. Women who test positive will be immediately discontinued from the study.

### Electrocardiogram

A single, standard 12-lead ECG was collected at Visit 1 prior to entering the No Drug Run-in Period of the study. The screening ECG was taken after the patient has been resting in the supine position for a minimum of 5 minutes. The ECGs were classified as normal, having a non-clinically significant abnormality, or having a clinically significantly abnormality. Any patients with clinically significant ECG abnormalities at Visit 1 were excluded.

### Vulvar-Vaginal Clinical Examination

A vulvar-vaginal clinical examination was performed using vulvoscopy to determine the level of irritation (using the scale below) at every visit. In addition, any patients who complain of local irritation or discomfort during the study were instructed to promptly contact the investigator for follow-up. This examination included a visual inspection of the vagina, including observations for petechiae, pallor, friability, dryness, and redness in the mucosa. Observations were rated on a 4-point scale (0, none; 1, mild; 2, moderate; 3, severe) as described in Table 5A.

TABLE 5A
Vulvoscopy - levels of irritation

| Reaction Type | 0 = None | 1 = Mild | 2 = Moderate | 3 = Severe |
|---|---|---|---|---|
| Erythema | None | Faint or mild redness | Moderate redness | Intense redness |
| | | of any size OR | involving >25% of | involving >25% of |
| | | moderate redness | the area OR intense | the area |
| | | involving ≤25% of | redness involving ≤25% | |
| | | the area | of the area | |
| Edema | None | Mild visible swelling | Moderate visible | Gross swelling or |
| | | or barely palpable | swelling OR easily | firm induration |
| | | edema of any size or | palpable edema | involving >25% of |
| | | easily palpable | involving >25% of | the area |
| | | edema involving ≤25% | the site or gross | |
| | | of the area | swelling of firm | |
| | | | induration involving ≤25% | |
| | | | of the area | |

### Statistical Analysis

A formal statistical analysis plan (SAP) was developed and finalized prior to unblinding the data. The SAP defined populations for analysis, outlined all data handling conventions, and specified all statistical methods to be used for analysis of the data. In general, safety and efficacy variables were presented by means of descriptive statistics and figures, as appropriate, by treatment arm and by time point, if applicable. Continuous variables were summarized using the number of non-missing observations, mean, standard deviation (SD), median, first and third quartiles, and minimum and maximum. Categorical variables were presented using the number of patients and percentages. For ordered categorical variables, cumulative percentages may also be presented. An unblinded ITT interim analysis was performed after at least 50 patients each have had their primary endpoints assessed on the Placebo and Sildenafil Cream arms. The interim analysis included all the patients who had successfully completed Visit 7 and those who drop out by the time of the interim analysis. Since it was not intended to stop the trial early for efficacy at this interim analysis, a conservative spending function based on Gamma family with parameter−40 was applied to each endpoint to compare the sildenafil 2 μm dose vs matching placebo at one-side 0.025 alpha level. The final study sample size in the Placebo and Sildenafil Cream arms was determined based on the interim conditional powers observed on the co-primary endpoints. For the sample size re-estimation in the Placebo and Sildenafil Cream arms, sample size will not be increased unless the interim conditional power is at least 50% on both co-primary endpoints, in which case it will be increased to the max of the two sample sizes calculated for each endpoint.

The sample size of this study was based on the co-primary product efficacy endpoints. The safety analysis set consisted of all women who applied IP at least once during the study. The frequency of TEAEs were described according to product group, relatedness to IP use or study procedures and severity. TEAE frequency was compared by treatment group using Chi square statistic or Fisher exact test, based on expected cell size. Vital sign data were normally distributed and therefore compared by visit and timing/position of the measurement (e.g., supine) between Sildenafil Cream, 3.6% and Placebo Cream groups using an independent samples t-test. P values<0.05 were considered significant.

### Efficacy Measures

The primary objective of the study was to demonstrate the efficacy of 2 grams of Sildenafil Cream, 3.6% compared to matching Placebo Cream based on the co-primary endpoints, SFQ-AS and Item 14 of the FSDS-DAO, as described below. Patient scores following the Single-Blind Placebo Run-In Period served as baseline for each patient.

Efficacy was evaluated using the following:

### Co-Primary Endpoints:

SFQ28 (AS): Change from baseline to end-of-study in the Arousal-Sensation (AS) domain score of the SFQ28
FSDS-DAO (Q14): Change from baseline to end-of-study in score for feeling concerned by difficulties with sexual arousal (Item 14 of the FSDS-DAO)

### Secondary Endpoint:

Arousal Diary (SSE): Change from baseline to end-of-study in number of Satisfactory Sexual Events (SSEs)

### Exploratory Endpoints:

Arousal Diary (SSE): In addition to the secondary endpoint analyses described above, change from baseline at Week 4 (Visit 5) and Week 8 (Visit 6) of the Double-Blind Dosing Period
Arousal Diary (AS): Change from baseline at Week 4 (Visit 5), Week 8 (Visit 6) and Week 12 (Visit 7) in patient reported genital arousal sensations (AS) captured by eDiary following each sexual event
Arousal Diary (AL): Change from baseline at Week 4 (Visit 5), Week 8 (Visit 6) and Week 12 (Visit 7) in patient reported lubrication (AL) captured by eDiary following each sexual event
Arousal Diary (GA): Change from baseline at Week 4 (Visit 5), Week 8 (Visit 6) and Week 12 (Visit 7) in patient reported sexual excitement (AE) captured by eDiary following each sexual event
SFQ28 (AS): In addition to the primary endpoint analyses described above, change from baseline at Week 4 (Visit 5) and Week 8 (Visit 6) of the Double-Blind Dosing Period.
SFQ28 (AL): Change from baseline at Week 4 (Visit 5), Week 8 (Visit 6) and Week 12 (Visit 7) in Arousal-Lubrication (AL) domain of the SFQ28
SFQ28 (AC): Change from baseline at Week 4 (Visit 5), Week 8 (Visit 6) and Week 12 (Visit 7) in Arousal-Cognitive (AC) domain of the SFQ28
SFQ28 (Desire): Change from baseline at Week 4 (Visit 5), Week 8 (Visit 6) and Week 12 (Visit 7) in desire domain of the SFQ28
SFQ28 (Orgasm): Change from baseline at Week 4 (Visit 5), Week 8 (Visit 6) and Week 12 (Visit 7) in orgasm domain of the SFQ28
FSDS-DAO: Change from baseline at Week 4 (Visit 5), Week 8 (Visit 6) and Week 12 (Visit 7) in FSDS-DAO total score
FSDS-DAO (Q14): In addition to the primary endpoint analyses described above, change from baseline in the FSDS-DAO Item 14 from Week 4 (Visit 5) and Week 8 (Visit 6) of the Double-Blind Dosing Period.
PGI-S: Proportion of patients who improved by at least one severity category from baseline to end-of-study
PGI-C: Proportion of patients who reported at least noticeable improvement related to problems with sexual arousal from baseline to the end-of-study.
PBE: Proportion of patients who reported experiencing meaningful benefit from study medication, assessed at the end-of-study with a Yes/No question

Unless otherwise stated, efficacy analyses was performed as randomized, i.e., a patient will be assigned to the treatment she was initially allocated, irrespective of what treatment was actually applied. Summary statistics were presented by treatment and time point (where applicable) for each efficacy measure.

The primary objective was assessed by testing the following hypotheses for the Sildenafil Cream, 3.6% treatment group (2 grams) versus the matching Placebo Cream for the SFQ28 (AS) endpoint and corresponding ones for the FSDS-DAO co-primary endpoint:
Null hypothesis (H0): There is no difference in the mean change from baseline to Week 12 in the SFQ28 (AS) for the Sildenafil Cream, 3.6% treatment group compared with the matching Placebo Cream arm.
H0: μ (Sildenafil)=μ (placebo)
Alternative hypothesis (H1): There is a difference in the mean change from baseline to Week 12 in the SFQ28 (AS) for the Sildenafil Cream, 3.6% treatment group compared with the matching Placebo Cream arm.
H1: μ (Sildenafil)+μ (placebo)

This comparison was carried out at the one sided 0.025% (or 2-sided 0.05) level of statistical significance using the fixed sequence fallback testing procedure. The analysis for Sildenafil Cream, 3.6% versus the matching Placebo Cream was conducted using a mixed model repeated measures (MMRM) model including terms for baseline (as a continuous covariate), treatment group, visit (as a categorical variable) and the interaction visit*treatment as fixed effects and subject as a random effect. An “unstructured” co-variance structure was used to model the within-subject errors. The Kenward-Roger approximation was used to estimate denominator degrees of freedom and adjust standard errors. The estimated treatment means and difference (Sildenafil Cream, 3.6% versus the matching Placebo Cream) at the last visit (Visit 7 of the Double-Blind Dosing Period) compared to baseline was reported as part of this analysis. The analysis will be performed on the ITT dataset. The analysis was also performed on the PP set.

### Results

Among the 252 participants who entered the 28-day single-blind placebo run-in period, 227 participants received at least one dose of IP and 219 of them completed the single-blind placebo run-in period. During this time, participants received placebo cream for a mean of 2.5 (1.3) weeks and an average of 4 doses. Median compliance with IP during the single-blind placebo run-in period was reported at 75%. In the single-blind period, 51 of 227 placebo exposed participants (22.5%) reported 94 TEAEs. All subjects who reported TEAEs during the single-blind placebo run-in period rated them as mild or moderate in severity. Of these 94 TEAEs, 73 TEAES, reported by 37 subjects, were graded as treatment-related TEAEs. Six treatment-related TEAEs, reported by four participants, led to discontinuation of IP during the single-blind placebo run-in period. All 6 of these TEAEs were symptoms which occurred shortly after application of IP including genital discomfort, burning, stinging, folliculitis, and erythema. During the single-blind placebo run-in period, the most common TEAE reported by 32 participants was genital application site discomfort, with COVID-19 infection being the second most common TEAE, reported by 12 participants.

During the single-blind placebo run-in period, 7 of 197 placebo exposed sexual partners reported 8 TEAEs, which were all mild to moderate in intensity. Four sexual partners reported 5 TEAEs (headache, penile burning, penile irritation, perineal body irritation) which were deemed treatment-related. There were no partner reported TEAEs which lead to IP discontinuation or study discontinuation during the single-blind placebo run-in period.

During the double-blind dosing period, the mean (standard deviation, SD) duration of IP use was 10.3 (2.5) and 9.7 (3.1) weeks for Sildenafil and Placebo users, respectively. The cumulative dose of Sildenafil and Placebo Creams received during this time was 23.5 (14.0) versus 22.9 (14.5) grams, respectively. Median reported compliance, defined as using IP 10-20 minutes prior to a sexual experience, ranged between 78.8-98% for Sildenafil Cream, 3.6% users versus 85-95% for Placebo Cream users. The highest median compliance rate for Sildenafil Cream 3.6% users (98%) was reported during the 3rd month of use, while the highest median compliance rate for Placebo Cream users was 95%, reported in the first month of use. During the double-blind dosing period, there were 1357 and 1160 sexual experiences recorded in which Sildenafil Cream, 3.6% or Placebo Cream was used, respectively. The majority of the sexual experiences were partnered (76% of Sildenafil Cream exposed events and 80% of Placebo Cream exposed sexual events). The eDiary showed no episodes of partnered sexual intercourse with an un-consented partner and therefore no participants were discontinued from the study for this protocol violation.

Table 5B shows the number of participants and sexual partners reporting at least one TEAE and the total number of TEAEs by severity and relatedness categories. There were no significant differences in the frequency of various TEAE reports by Sildenafil Cream, 3.6% versus Placebo Cream users in the double-blind dosing period (all p values>0.50, Table 5B). The mean (SD) time of onset of the first TEAE reported during double-blind dosing period was 21 (11) and 20 (8) days for Sildenafil and Placebo Cream assigned participants, respectively. All TEAEs reported by participants or sexual partners during the double-blind dosing period were graded as mild or moderate. During the dosing period, 2 Sildenafil assigned participants reported 3 TEAEs (burning at IP application site, erythema at IP application site and dyspareunia with IP use) which were all deemed to be treatment related and led to discontinuation of IP. There were 2 TEAEs reported by Placebo Cream users in the double-blind treatment period which were not related to IP use (dyspareunia and vulvar fissure) but led to IP discontinuation nonetheless.

Table 5B also demonstrates that during the double-blind dosing period, 7 partners exposed to Sildenafil Cream, 3.6% reported 9 TEAEs and 4 partners exposed to Placebo Cream reported 4 TEAEs. There were no statistically significant differences in the frequency of various TEAE reports by sexual partners exposed either to active or placebo IP (all p values>0.25). There was one TEAE, genital burning upon contact with IP during intercourse, which was considered treatment related and led to IP discontinuation in a Sildenafil Cream, 3.6% exposed sexual partner.

TABLE 5B
TEAEs Reported by Participants and Sexual Partners During Double Blind Treatment Period

| | Sildenafil (N = 99 Participants; | | | Placebo (N = 94 Participants; | | | |
|---|---|---|---|---|---|---|---|
| | N = 91 Partners) | | | N = 84 Partners) | | | |
| | | % of | | | % of | | |
| | N Participants | Cohort | | N Participants | Cohort | | |
| | Reporting | Reporting | N | Reporting | Reporting | N | Fisher |
| TEAE Event | at Least 1 | at Least 1 | TEAEs | at Least 1 | at Least 1 | TEAEs | Exact |
| Category | TEAE | TEAE | Reported | TEAE | TEAE | Reported | P value |

PARTICIPANT Treatment Emergent Adverse Event Data

| | | | | | | | |
|---|---|---|---|---|---|---|---|
| Any TEAE | 29 | 29.3 | 78 | 28 | 29.8 | 65 | 0.76 |
| Treatment | 14 | 14.1 | 60 | 14 | 14.9 | 37 | >0.99 |
| Related TEAE | | | | | | | |
| TEAE leading to | 2 | 2.0 | 3 | 2 | 2.1 | 2 | >0.99 |
| discontinuation | | | | | | | |
| of Investigational | | | | | | | |
| Product (IP) | | | | | | | |
| Treatment | 2 | 2.0 | 3 | 1 | 1.1 | 1 | 0.50 |
| Related TEAE | | | | | | | |
| leading to | | | | | | | |
| discontinuation | | | | | | | |
| of IP | | | | | | | |
| Any SAE | 0 | 0 | 0 | 0 | 0 | 0 | NA |

PARTNER Treatment Emergent Adverse Event Data

| | | | | | | | |
|---|---|---|---|---|---|---|---|
| Any TEAE | 7 | 7.7 | 9 | 4 | 4.8 | 4 | 0.54 |
| Treatment | 3 | 3.3 | 5 | 0 | 0 | 0 | 0.25 |
| Related TEAE | | | | | | | |
| TEAE leading to | 1 | 1.1 | 1 | 0 | 0 | 0 | >0.99 |
| discontinuation | | | | | | | |
| of Investigational | | | | | | | |
| Product (IP) | | | | | | | |
| Treatment | 1 | 1.1 | 1 | 0 | 0 | 0 | >0.99 |
| related TEAE | | | | | | | |
| leading to | | | | | | | |
| discontinuation | | | | | | | |
| of IP | | | | | | | |

A post-hoc analysis (Table 5C) demonstrated that the number of Sildenafil Cream, 3.6% assigned participants reporting at least one type of TEAE was similar, when sub-categorized based on the quartile of total number of reported sexual events during the double-blind treatment period (all p values>0.19).

TABLE 5C
Number of Sildenafil Cream 3.6% assigned participants reporting at least one
TEAE category based on quartile of sexual events reported during the double-blind
treatment period

| TEAE | | | | | | | | | |
|---|---|---|---|---|---|---|---|---|---|
| Variable | Quartile of Sexual Events Recorded During the 12-week Double- | | | | | | | | |
| (Number of | Blind Treatment Period | | | | | | | | |
| Participants | 1^{st} Quartile (1-7 | | 2nd Quartile (8-11 | | 3rd Quartile (12-18 | | 4th Quartile (9-45 | | |
| Reporting at | sexual events) | | sexual events) | | sexual events) | | sexua events) | | Fisher |
| Least One of | (N = 29) | | (N = 23) | | (N = 24) | | (N = 25) | | Exact |
| the TEAE | | (% | | (% | | (% | | (% | P |
| Variable Type* | N | GROUP) | N | GROUP) | N | GROUP) | N | GROUP) | Value |

TEAE SEVERITY

| | | | | | | | | | |
|---|---|---|---|---|---|---|---|---|---|
| MILD | 4 | 13.8% | 7 | 30.4% | 3 | 12.5% | 2 | 8.0% | 0.19 |
| MODERATE | 6 | 20.7% | 1 | 4.3% | 4 | 16.7% | 2 | 8.0% | |

TEAE RELATEDNESS

| | | | | | | | | | |
|---|---|---|---|---|---|---|---|---|---|
| RELATED | 7 | 24.1% | 3 | 13.0% | 2 | 8.3% | 2 | 8.0% | 0.35 |
| NOT | 3 | 10.3% | 5 | 21.7% | 5 | 20.8% | 2 | 8.0% | |
| RELATED | | | | | | | | | |

PRODUCT DISCONTINUED

| | | | | | | | | | |
|---|---|---|---|---|---|---|---|---|---|
| YES | 2 | 6.9% | 0 | 0% | 0 | 0% | 0 | 0% | 0.49 |
| NO | 8 | 27.6% | 8 | 34.8% | 7 | 29.2% | 4 | 16.0% | |

STUDY DISCONTINUED

| | | | | | | | | | |
|---|---|---|---|---|---|---|---|---|---|
| YES | 2 | 6.9% | 0 | 0% | 0 | 0% | 0 | 0% | 0.49 |
| NO | 8 | 27.6% | 8 | 34.8% | 7 | 29.2% | 4 | 16.0% | |

*For participants with more than one TEAE, the highest severity, relatedness and action was recorded.

Table 5D displays the most common, treatment-related (possibly, probably, or definitely related) TEAEs, by system organ class, reported by subjects or their sexual partners during the double-blind dosing period. As noted in Table 5D, there were no significant differences in the system organ class of treatment related TEAEs reported by Sildenafil versus Placebo Cream users (all p values>0.11) nor their sexual partners (all p values>0.25).

TABLE 5D
Most Common Treatment-Related TEAEs by System Organ Class, Reported
by Participants and Sexual Partners During Double-Blind Dosing Period

| | Sildenafil | | Placebo | | |
|---|---|---|---|---|---|
| | (N = 99 Participants; | | (N = 94 Participants; | | |
| | N = 91 Partners) | | N = 84 Partners) | | |
| | N Number | % of Total | N Number | % of Total | |
| | of Patients | Group | of Patients | Group | Fisher |
| | Reporting | Reporting | Reporting | Reporting | Exact |
| System Organ Class | at Least | at Least | at Least | at Least | P |
| Preferred Term | One TEAEs | One TEAE | One TEAEs | One TEAE | value |

PARTICIPANT Treatment Related TEAE Data

| | | | | | |
|---|---|---|---|---|---|
| Any Treatment Related | 14 | 14.1 | 14 | 14.9 | >0.99 |
| Adverse Events | | | | | |
| Application Site | 12 | 12.1 | 14 | 14.9 | 0.67 |
| Discomfort | | | | | |
| Application Site | 3 | 3.0 | 3 | 3.2 | >0.99 |
| Erythema | | | | | |
| Application Site | 2 | 2.0 | 1 | 1.1 | >0.99 |
| Edema | | | | | |
| Vulvovaginal | 0 | 0 | 2 | 2.1 | 0.24 |
| Discomfort | | | | | |
| Cervical Friability, | 0 | 0 | 3 | 3.9 | 0.11 |
| Dysmenorrhea or | | | | | |
| Dyspareunia | | | | | |
| Vulvovaginal | 1 | 1.0 | 0 | 0 | >0.99 |
| Erythema | | | | | |

PARTNER Treatment Related TEAE Data

| | | | | | |
|---|---|---|---|---|---|
| Any Treatment Related | 3 | 3.1 | 0 | 0 | 0.25 |
| TEAE | | | | | |
| Application Site | 1 | 1.1 | 0 | 0 | >0.99 |
| Discomfort | | | | | |
| Headache | 1 | 1.1 | 0 | 0 | >0.99 |
| Genital Hypothesia | 1 | 1.1 | 0 | 0 | >0.99 |

Because oral sildenafil use can be associated with orthostatic hypotension, particularly in users taking nitrates for angina, orthostatic vital signs were obtained at baseline and then monthly during the double-blind dosing period among participants (Table 6) and their sexual partners. As noted in Table 6, there were no differences in vital signs taken at various positions between Sildenafil Cream, 3.6% or Placebo assigned participants at any point in the study (all p values>0.10). Consistent with normal physiologic response, mean pulse rate increased from that recorded in the supine position, at 1 minute and 3 minutes standing. The median change in pulse rate from baseline measurements increased by 1-4 beats/minute in the Sildenafil Cream, 3.6% and Placebo groups with no outliers noted. For systolic blood pressure, median changes from baseline measurements in both treatment groups ranged from −3.5 mmHg to +3.0 mmHg, showing no clinically significant changes. Finally, diastolic blood pressure showed a median change of −2.0 to +1.0 from baseline during the double-blind dosing period.

During the double-blind dosing period, one Placebo randomized subject reported heart palpitations which were mild in severity and deemed to be unrelated to study IP. There were no other cardiovascular or nervous system disorders reported by subjects during the double-blind dosing period. For sexual partners, during the double-blind dosing period, one Sildenafil Cream, 3.6% exposed partner reported a headache that was graded as mild but deemed to be related to study IP exposure. There were no other cardiovascular or nervous system TEAEs reported by sexual partners during the double-blind dosing period.

Finally, although baseline laboratories and electrocardiograms were not repeated at the end of the study, there were no TEAEs which necessitated repeating these baseline screening assessments.

TABLE 6
Participant Orthostatic Vital Sign Data by Visit and Product Assignment

| | Vital Sign | | | | | | | |
|---|---|---|---|---|---|---|---|---|
| | Measurement | Sildenafil (N = 99) | | | Placebo (N = 134) | | | P |
| Visit | Condition | N | Mean | SD | N | Mean | SD | value |

Pulse Rate (beats/min)

| | | | | | | | | |
|---|---|---|---|---|---|---|---|---|
| Start of Double- | Supine | 99 | 71.4 | 11.12 | 115 | 72.2 | 10.84 | 0.83 |
| Blind Treatment | 1 Minute Post | 99 | 78.4 | 11.48 | 115 | 78.8 | 11.26 | 0.92 |
| Period Visit 4 | Standing | | | | | | | |
| | 3 Minutes Post | 99 | 79.5 | 11.11 | 115 | 78.7 | 10.00 | 0.47 |
| | Standing | | | | | | | |
| 4 Weeks Post | Supine | 99 | 71.6 | 11.01 | 94 | 72.4 | 10.52 | 0.53 |
| Double-Blind | 1 Minute Post | 99 | 78.0 | 10.43 | 94 | 78.4 | 10.27 | 0.66 |
| Treatment Visit 5 | Standing | | | | | | | |
| | 3 Minutes Post | 99 | 79.6 | 10.68 | 94 | 79.5 | 10.05 | 0.88 |
| | Standing | | | | | | | |
| 8 Weeks Post | Supine | 94 | 71.8 | 11.20 | 85 | 72.8 | 10.02 | 0.55 |
| Double-Blind | 1 Minute Post | 94 | 78.8 | 10.23 | 85 | 80.1 | 11.55 | 0.41 |
| Treatment Visit 6 | Standing | | | | | | | |
| | 3 Minutes Post | 94 | 79.4 | 10.32 | 85 | 80.1 | 10.54 | 0.64 |
| | Standing | | | | | | | |
| 12 Weeks Post | Supine | 91 | 71.0 | 10.14 | 85 | 73.3 | 10.50 | 0.15 |
| Double-Blind | 1 Minute Post | 91 | 79.1 | 11.49 | 85 | 79.9 | 11.26 | 0.63 |
| Treatment Visit 7 | Standing | | | | | | | |
| | 3 Minutes Post | 91 | 78.3 | 10.08 | 85 | 80.4 | 10.70 | 0.18 |
| | Standing | | | | | | | |

Systolic Blood Pressure (mmHg)

| | | | | | | | | |
|---|---|---|---|---|---|---|---|---|
| Start of Double- | Supine | 99 | 114.9 | 10.33 | 115 | 116.2 | 11.60 | 0.31 |
| Blind Treatment | 1 Minute Post | 99 | 115.4 | 11.29 | 115 | 116.8 | 11.40 | 0.33 |
| Period Visit 4 | Standing | | | | | | | |
| | 3 Minutes Post | 99 | 115.3 | 10.63 | 114 | 116.7 | 11.95 | 0.37 |
| | Standing | | | | | | | |
| 4 Weeks Post | Supine | 99 | 115.4 | 10.96 | 94 | 114.5 | 11.97 | 0.38 |
| Double-Blind | 1 Minute Post | 99 | 116.3 | 11.46 | 94 | 115.7 | 11.74 | 0.60 |
| Treatment Visit 5 | Standing | | | | | | | |
| | 3 Minutes Post | 99 | 115.5 | 10.78 | 94 | 114.6 | 11.57 | 0.49 |
| | Standing | | | | | | | |
| 8 Weeks Post | Supine | 94 | 116.4 | 11.49 | 85 | 115.4 | 11.57 | 0.58 |
| Double-Blind | 1 Minute Post | 94 | 116.8 | 11.30 | 85 | 117.1 | 11.32 | 0.85 |
| Treatment Visit 6 | Standing | | | | | | | |
| | 3 Minutes Post | 94 | 116.6 | 11.21 | 85 | 116.7 | 10.60 | 0.94 |
| | Standing | | | | | | | |
| 12 Weeks Post | Supine | 91 | 115.8 | 11.43 | 85 | 117.4 | 12.74 | 0.39 |
| Double-Blind | 1 Minute Post | 91 | 115.7 | 12.34 | 85 | 117.6 | 12.80 | 0.32 |
| Treatment Visit 7 | Standing | | | | | | | |
| | 3 Minutes Post | 91 | 115.9 | 11.45 | 85 | 117.2 | 12.96 | 0.47 |
| | Standing | | | | | | | |

Diastolic Blood Pressure (mmHg)

| | | | | | | | | |
|---|---|---|---|---|---|---|---|---|
| Start of Double- | Supine | 99 | 75.1 | 7.85 | 115 | 75.6 | 7.39 | 0.41 |
| Blind Treatment | 1 Minute Post | 99 | 79.4 | 8.06 | 115 | 78.7 | 7.03 | 0.89 |
| Period Visit 4 | Standing | | | | | | | |
| | 3 Minutes Post | 99 | 79.7 | 7.60 | 114 | 79.5 | 8.21 | 0.99 |
| | Standing | | | | | | | |
| 4 Weeks Post | Supine | 99 | 75.4 | 7.78 | 94 | 75.7 | 7.70 | 0.86 |
| Double-Blind | 1 Minute Post | 99 | 79.3 | 6.98 | 94 | 78.7 | 8.19 | 0.49 |
| Treatment Visit 5 | Standing | | | | | | | |
| | 3 Minutes Post | 99 | 79.6 | 7.25 | 94 | 78.7 | 8.13 | 0.37 |
| | Standing | | | | | | | |
| 8 Weeks Post | Supine | 94 | 75.8 | 8.15 | 85 | 75.8 | 7.85 | 0.96 |
| Double-Blind | 1 Minute Post | 94 | 79.4 | 7.31 | 85 | 79.5 | 7.65 | 0.90 |
| Treatment Visit 6 | Standing | | | | | | | |
| | 3 Minutes Post | 94 | 79.5 | 7.63 | 85 | 78.8 | 7.13 | 0.54 |
| | Standing | | | | | | | |
| 12 Weeks Post | Supine | 91 | 75.9 | 7.26 | 85 | 76.1 | 8.37 | 0.88 |
| Double-Blind | 1 Minute Post | 91 | 79.6 | 7.15 | 85 | 79.0 | 8.11 | 0.59 |
| Treatment Visit 7 | Standing | | | | | | | |
| | 3 Minutes Post | 91 | 80.4 | 7.92 | 85 | 78.4 | 8.14 | 0.10 |
| | Standing | | | | | | | |

### Conclusions

This study has shown that topical Sildenafil Cream, 3.6% was safe and well tolerated by healthy premenopausal women and their sexual partners in 1357 documented Sildenafil Cream 3.6% sexual exposures in the double-blind treatment period. Previous clinical trials of oral sildenafil citrate in women had high rates of side effects using doses normally administered to men for ED (25 mg-100 mg).

The most common side effects of oral sildenafil use are headache, flushing, dyspepsia, abnormal vision, nasal congestion, back pain, myalgia, nausea, dizziness and rash.

The safety data presented herein support that the side effects associated with topical Sildenafil Cream, 3.6% use were primarily related to local genital irritation. Post-hoc analysis found that the frequency of TEAEs was similar based on the quartile of total reported sexual exposures during the double-blind treatment period, supporting no cumulative toxicity from multiple exposures. Many Sildenafil Cream, 3.6% users used the product more than 9 times per month, as was specified in the protocol, without an increase in TEAEs for these protocol violations. Although the sample size was not powered to detect differences in TEAEs, significant differences in the types of TEAEs reported by participants using Sildenafil Cream, 3.6% versus Placebo were not found, using exact statistical methods. This study also described TEAEs among sexual partners, and TEAEs were reported directly by the sexual partner, rather than being reported on their behalf by study participants, which is often the case in clinical trials of products used with sexual intercourse.

Strengths of this study included the detailed reporting of TEAEs by participants, via an eDiary with daily prompts and direct TEAE reporting among sexual partners as noted above. Based on reluctance on the part of potential volunteers to involve their sexual partners in the clinical study a protocol amendment allowed un-partnered women or women whose partners did not want to participate in the informed consent or safety monitoring process to enroll. Consistent with good clinical practice (GCP), participants with un-consented sexual partners were instructed to not have a partnered sexual experience within 72 hours of IP application. Given that the sexual partner TEAEs were infrequent and all mild or moderate severity, the potential benefit of topical sildenafil outweighs the risks to users and their sexual partners.

Postural vital signs were monitored in the clinic and or via telemedicine for sexual partners but could not measure postural vital signs immediately after IP use. Data from the initial safety studies in men, discussed above, support that changes in heart rate with standing were likely benign, normal, physiologic response to standing, and the ample vital sign and TEAE data collected in this study show that the systemic impacts of oral sildenafil citrate were not seen with topical application.

In conclusion, these data support that topical sildenafil is safe among users and exposed sexual partners. These data support clinical development of Sildenafil Cream, 3.6% for treatment for FSAD.

### Example 3: Exit Interviews in Female Sexual Arousal Disorder-Addendum to Phase 2B Study

To evaluate patient-reported outcome measures (PROMs) for use in the assessment of efficacy in FSAD patients, interviews with pre- and post-menopausal women with FSAD age 21-70 were conducted to support content the PROMs.

The results of the study confirmed the PROMs are fit-for-purpose, assess concepts in FSAD and provide support for the instruments to be used as endpoints in clinical trials of FSAD patients. Exit Interviews are performed to better understand what would be considered a meaningful change on the measures to be used as primary or secondary endpoints. Also, to ascertain what patients are considering if they rate change as improved on the Patient Global Impression of Change (PGI-C), have an improvement in severity by 1 or 2 categories on the Patient Global Impression of Severity (PGI-S) or report meaningful benefit from study medication on the Patient Benefit Evaluation (PBE).

Exit Interviews will be conducted with a subset of patients at the end of the study (at Visit 7).

The overall objective of conducting this research is to further evaluate, according to the FSAD patient perspective:
what constitutes meaningful change for the following patient-reported outcome measures (PROMs):
   SFQ-AS domain,
   FSDS-DAO item 14, and
   Arousal Diary AS domain
what is being considered when responding ‘yes’ or ‘no’ to the patient benefit evaluation question

### Study Design

This interview study will focus on data collection from a subset of women participating in the Phase 2b FSAD clinical trial described in Example 2. This study is designed as a sub-study that will be an addendum to the main clinical trial protocol. The Exit Interview will be conducted via videoconference or telephone at clinical trial completion for each patient willing and eligible to participate. Interviews will focus on discussing with patients what constitutes a meaningful change on the PROMs of interest (SFQAS domain, AS Diary domain and FSDS-DAO item 14, PBE) and what constitutes improvement on the PGI-C and PGI-S.

### Study Population

In total, 60 adult (age>21) women with FSAD will be recruited from the Phase 2b clinical trial of Example 2, which will enroll approximately 400 to 590 (300 to 400 to complete) patients. The women selected to participate in the Exit Interview will be those who complete the study first and have agreed to be a part of this subset. The sample size is driven by the requirement to have a mix of differing levels of improvement observed over the course of the clinical trial.

### Exit Interview Procedure

Given that the Exit Interviews will take place up to two weeks after the last clinical trial visit, the interviewer will establish if: (a) any changes occurred during the trial period; and (b) if any changes occurred between the last visit and now (i.e., interview day). This will help ensure that the interview discussion is focused on the patient's experience during the trial and not how the patient has felt since their last visit (should any symptoms have improved or worsened since stopping study treatment).

### Part 1: Overall FSAD Experiences

Introduction and discussion about experience of living with FSAD. The purpose of this section is to “warm-up” the patient and make them feel relaxed rather than to explore patient experience in depth. Therefore, questions in this section are brief and discussion should last approximately 10 minutes.

Part 2: Experience of FSAD-Related Issues with Sexual Arousal or Sensation

As the interview continues, questions will focus on issues around sexual arousal and sensation, and experiences of change during the trial (for example, if these have improved, stayed the same or worsened, and whether any of these changes were meaningful).

Patients will be asked about whether greater/lesser score change on each PROM instruments response scale would be meaningful, and what changes symptoms or impacts are reflected by each level of score change.
Any changes over time with treatment in each response scale, and MCT of each response scale, will be discussed for the following items:
   Four (4) items from SFQ28 AS domain
   FSDS-DAO item 14
   Five (5) items from Arousal diary AS domain
   Single (1) item PGI-S
   Single (1) item PGI-C
   Single (1) item PBE (i.e., Patient response of Yes/No to the question “Did you experience meaningful benefit from the study medication?”)
This line of questioning will last approximately 45 minutes.
   Part 3: Patient Opinion about Clinical Trial Topical Medication
If time permits, there will be discussion about the use of the topical agent e.g., application process of the cream, ease of use, texture, and if the patients would recommend it to a friend. This discussion will take approximately 5 minutes.

### Example 4: Evaluate the Experience of Condition-Associated Symptoms and the Relative Importance of FSAD Symptoms

### Objectives

The aim of this study was to conduct in-depth, qualitative interviews with premenopausal (n=20) and postmenopausal women (n=15) who were clinically diagnosed with FSAD (N=35) by a trained sexual medicine clinician. The objective of the interview was to evaluate the experience of condition-associated symptoms and the relative importance of FSAD symptoms including their severity, bother, and impact on participants' health-related quality of life.

### Methods

### Study Population

Adult, female participants between the ages of 21-70 who had a clinical diagnosis of FSAD were recruited to participate in this study. Participants were required to be sexually active (in the past 4 weeks) and have previously experienced “normal” sexual function for at least 2 years or longer. Participants who met all of the inclusion and exclusion criteria underwent a formal, semi-structured clinical interview to confirm the diagnosis of FSAD as defined by the DSM-IV-TR as the primary FSD complaint. FSAD is categorized by the DSM-IV-TR as acquired versus lifelong and generalized versus situational, with symptoms which have been present for at least the past 6 months prior to the clinical interview. Eligible participants had a primary complaint of lack of genital arousal during sexual activity, and this genital response had a detrimental and distressing impact on their overall sexual experience.

### Recruitment

Women were recruited via an internet-based screening questionnaire and clinically diagnosed with FSAD. Prior to Visit 1, initial eligibility was assessed on the phone. If the participant was determined to be potentially eligible, informed consent was administered and completed by site staff at an initial visit to confirm diagnosis (Visit 1). Following consent, the inclusion/exclusion criteria were confirmed, and a clinical interview was performed to confirm a primary diagnosis of FSAD as defined by the DSM-IV-TR. Women who had secondary complaints related to low desire or problems with orgasm due to lack of genital arousal (HSDD) or cognitive arousal symptoms (i.e., not feeling aroused mentally during sexual activity) were considered eligible as long as symptoms related to diminished genital arousal were deemed the most bothersome symptom and developed before low desire. If deemed eligible after Visit 1, women were asked to return for a second visit (Visit 2) to take part in an in-depth, qualitative, one-to-one, 90-minute interview.

### Study Interview Visit

A combined concept elicitation (CE) and cognitive debriefing (CD) semi-structured interview study was identified as the most suitable approach to address the research objectives (only the CE portion of the interview is reported here). Semi-structured interviews allow for the exploration of participants' subjective thoughts, experience, and details that would not be possible with structured interviews. In addition, semi-structured interviews allow for the evaluation of participants' responses about specific symptoms by use of pertinent probes where necessary to facilitate discussion that would not be possible in an unstructured interview.

The concept elicitation interview studied the symptoms and impacts experienced by women who have FSAD. Open-ended questions were used in order to encourage the participant to spontaneously report on their experiences. When needed, more direct questioning was employed by the interviewer to elicit greater detail from the participant. The following are examples of the types of questions asked:
‘Tell me about the typical arousal problems that you experience’,
‘How long has this been happening’, ‘how bad can this problem be’, ‘How does this problem make you feel when it happens’,
‘How does the problem make you feel about being intimate with your partner’,
‘Thinking about the different types of things you've mentioned, can you rank them from least to most bothersome’.

This data collection approach was used to generate participant-driven insights and to allow for a bottom-up, thematic analysis of the data.

### Analysis

All interviews were audio-recorded and transcribed verbatim by a professional transcription company (Rev) into a Microsoft Word document that was used for analysis. Nvivo v12.0, a qualitative software program, was utilized to assist researchers with the coding and analysis of the qualitative data. This software allows researchers to apply codes to sections of text using thematic analysis. Thematic analysis allowed the four expert researchers who coded the data to identify themes or patterns within the data. Symptoms and impacts were coded as “spontaneous(S)” or “probed (P)” at the first point of mention during the interview to provide an assessment of which symptoms and impacts were most frequently described by participants without prompting by the interviewer. A codebook was developed to assist with coding that the four expert coders then used to independently code the transcripts.

All key concepts had emerged in the interview sample (referred to as a “saturation analysis”). Researchers assessed whether concept saturation had been obtained in that no new themes or descriptions of concepts were being introduced in the final set of interviews. To determine this, the transcripts were grouped into three sets for the premenopausal and postmenopausal cohorts, in the sequential order in which they were performed. Concepts elicited were then compared between sets. The point at which no new concepts emerged was the point at which saturation was deemed to have been achieved.

### Results

### Study Population

A total of n=23 premenopausal and n=13 postmenopausal women participated in the study. The n=23 premenopausal and n=13 postmenopausal women were predominantly white (60.9%/69.2%; Pre/Post). Black/African American was the next highest race reported by six women, with an even split between the pre- and postmenopausal samples. Four women in the premenopausal group identified as Hispanic/Latino. The overall average age was 49 years old with an age range of 23 to 69. The majority of participants (n=17) had a college education, 10 of which also had a graduate degree. Almost half of the population (n=18) reported full-time employment as their work status. The most reported relationship status for both pre- and postmenopausal samples was married (n=16), followed by “in a committed relationship with one person” (n=11). 94.4% of women considered their sexual orientation to be heterosexual while 5.6% were bisexual.

### Interview Results

### Sexual Activity

At the beginning of the interviews, women were asked about the types of sexual activity in which they regularly engage. Women were asked to spontaneously describe and define these activities. Interviewers also probed using a pre-determined list of activities which included intercourse, caressing, foreplay, masturbation, and oral sex. All premenopausal women reported participating in sexual intercourse, which was defined by n=9 participants using the words “penetration,” “penetrate,” “penetrative sex,” or “penis in vagina.” Most premenopausal women also reported participating in oral sex (n=18) and masturbation (n=15), in which the mentions were nearly split between spontaneous and probed responses. Anal sex was reported by n=2 premenopausal participants spontaneously.

Nearly all postmenopausal women reported participating in sexual intercourse (n=11), which was defined by n=9 participants using the words “penetration,” “penetrate,” “penetrative sex,” or “penis in vagina.” Most postmenopausal women also reported participating in oral sex (n=8) and masturbation (n=10). All postmenopausal participants who mentioned oral sex did so spontaneously, but for masturbation the split was nearly equal between spontaneous and probed responses. Anal sex was reported by n=3 postmenopausal participants, all spontaneously. In both the premenopausal and postmenopausal groups, caressing and foreplay were not spontaneously reported by as many women as intercourse, oral sex, and masturbation.

### Frequency of Symptoms

Following the question about sexual activity, women were asked, “What physical/genital sensations or feelings do you experience now” in order to elicit spontaneous responses about the types of sexual symptoms they experience as a result of FSAD. Premenopausal women spontaneously mentioned ten symptoms, eight of which were physical/genital arousal focused and two of which were related to lack of cognitive arousal (“not feeling turned on” or “excited”) and low sexual desire. Postmenopausal women spontaneously mentioned eight symptoms, seven of which were physical/genital arousal focused and one of which was related to lack of sexual desire (Table 7).

The most frequently reported symptom in the premenopausal (n=21; n=11 spontaneously, n=10 probed) and postmenopausal (n=13; n=7 spontaneously, n=6 probed) group was “inability or difficulty with orgasm.”

Lack of lubrication or wetness was the next most frequently reported symptom in the premenopausal group (n=19; n=13 spontaneously, n=6 probed), followed by loss of feeling/sensation (n=17; n=13 spontaneously, n=6 probed). In the postmenopausal group, lack of lubrication or wetness and loss of feeling/sensation were the next most frequently reported symptoms (loss of lubrication/wetness n=12; n=7 spontaneously, n=5 probed; loss of feeling/sensation n=12; n=11 spontaneously, n=1 probed).

Although inability or difficulty with orgasm was the highest reported symptom overall, lack of lubrication or wetness and loss of feeling/sensation in the premenopausal group (spontaneously reported by n=13) and loss of feeling/sensation in the postmenopausal group (spontaneously reported by n=11) were mentioned by more participants spontaneously than those who endorsed inability or difficulty with orgasm.

Women also discussed some of the consequences of diminished physical/genital arousal during sexual activity, i.e., low desire, cognitive arousal (“not feeling turned on”). Specifically, cognitive arousal and sexual desire impacts were discussed as a result of genital arousal symptoms: desire: premenopausal women: n=12 (n=11 spontaneously, n=1 probed); postmenopausal women: n=7, all spontaneously reported; cognitive-arousal issues: premenopausal women: n=12 (n=5 spontaneously, n=7, probed); postmenopausal women: n=4, all probed. Due to the fact that no postmenopausal participants reported “not feeling excited” as a symptom, the concept “not feeling turned on/excited” which was included in the premenopausal cohort is listed only as “not feeling turned on” in the postmenopausal cohort.

TABLE 7
Frequency of Participant-Reported FSAD Symptoms in Pre- and Postmenopausal
Women

| | | Premenopausal | | | Postmenopausal | | |
|---|---|---|---|---|---|---|---|
| | Sub- | Spontaneous | Probed | Total | Spontaneous | Probed | Total |
| Concept | Concepts | (n = 23) | (n = 23) | (N = 23) | (n = 13) | (n = 13) | (N = 13) |
| Physical/ | Difficulty or | 11 | 10 | 21 | 7 | 6 | 13 |
| Genital | inability to | (48%) | (43%) | (91%) | (54%) | (46%) | (100%) |
| Arousal | orgasm | | | | | | |
| | Lack of | 13 | 6 | 19 | 7 | 5 | 12 |
| | lubrication or | (57%) | (26%) | (83%) | (54%) | (38%) | (92%) |
| | wetness | | | | | | |
| | Loss of | 13 | 4 | 17 | 11 | 1(8%) | 12 |
| | feeling/ | (57%) | (17%) | (74%) | (85%) | | (92%) |
| | sensation | | | | | | |
| | Lack of | 8 | 4 | 12 | 3 | 1(8%) | 4 |
| | tingling | (35%) | (17%) | (52%) | (23%) | | (31%) |
| | Loss of | 6 | 3 | 9 | 1 | 1(8%) | 2 |
| | feelings of | (26%) | (13%) | (39%) | (8%) | | (15%) |
| | fullness/ | | | | | | |
| | engorgement | | | | | | |
| | Lack of | 6 | 2 | 8 | 3 | 2 | 5 |
| | warmth | (26%) | (9%) | (35%) | (23%) | (15%) | (38%) |
| | Lack of | 4 | 1 | 5 | 0 | 2 | 2 |
| | throbbing | (17%) | (4%) | (22%) | (0%) | (15%) | (15%) |
| | Lack of | 1 | 0 | 1 | 0 | 4 | 4 |
| | pulsating | (4%) | (0%) | (4%) | (0%) | (31%) | (31%) |
| | Discomfort | — | — | — | 2 | 0(0%) | 2 |
| | | | | | (15%) | | (15%) |
| Cognitive | Low desire | 11 | 1 | 12 | 7 | 0(0%) | 7 |
| Arousal/ | | (48%) | (4%) | (52%) | (54%) | | (54%) |
| Desire | Not feeling | 5 | 7 | 12 | 0 | 4 | 4 |
| | turned on or | (22%) | (30%) | (52%) | (0%) | (31%) | (31%) |
| | excited* | | | | | | |

*For the postmenopausal group, this concept is “not being turned on.”

### Most Problematic Symptoms

Participants in the premenopausal and postmenopausal cohorts were asked to rank their FSAD symptoms from most to least bothersome in order to reveal those which were the most problematic (Table 8). In the premenopausal group, the symptom reported most often in the top three was lack of lubrication or wetness (n=17), followed by loss of feeling/sensation (n=12), and then by inability to orgasm (n=8). Lack of lubrication was ranked as the most bothersome symptom by n=6 participants, and loss of feeling/sensation was ranked as the top symptom by n=5 participants. Difficulty or inability to orgasm followed lack of lubrication and loss of feeling/sensation, with eight women putting this in their top three symptoms. Lack of tingling (n=6) and not feeling turned on/excited were reported in the top three by n=7 and n=8 participants respectively. In the postmenopausal group, the symptom reported most often in the top three was loss of feeling/sensation (n=10), followed by lack of lubrication or wetness (n=9), and then by both the inability to orgasm and lack of desire (both n=6). Not feeling turned on was only noted by n=2 women as a top-most difficult symptom. Specific descriptors of loss of sensation such as tingling and throbbing were mentioned by one woman each, both reporting this as the third most bothersome symptom.

TABLE 8
Most Problematic FSAD Symptoms Reported by Pre- and Postmenopausal Women

| Symptoms | | | | | | | | |
|---|---|---|---|---|---|---|---|---|
| Identified as | Premenopausal | | | | Postmenopausal | | | |
| Most | #1 | #2 | #3 | Total | #1 | #2 | #3 | Total |
| Problematic* | (n = 23) | (n = 23) | (n = 23)** | (N = 23) | (n = 13)*** | (n = 13) | (n = 13)** | (N = 13) |
| Lack of | 6 | 6 | 5 | 17 | 3 | 2 | 4 | 9 |
| lubrication or | (26%) | (26%) | (22%) | (74%) | (23%) | (15%) | (31%) | (69%) |
| wetness | | | | | | | | |
| Loss of feeling/ | 5 | 5 | 2 | 12 | 2 | 6 | 2 | 10 |
| sensation | (22%) | (22%) | (9%) | (52%) | (15%) | (46%) | (15%) | (77%) |
| Difficulty or | 4 | 3 | 1 | 8 | 4 | 1 | 1 | 6 |
| inability to | (17%) | (13%) | (4%) | (35%) | (31%) | (8%) | (8%) | (46%) |
| orgasm | | | | | | | | |
| Lack of tingling | 2 | 5 | 0 | 7 | 0 | 0 | 1 | 1 |
| | (9%) | (22%) | (0%) | (30%) | (0%) | (0%) | (8%) | (8%) |
| Not feeling | 3 | 1 | 2 | 6 | 2 | 0 | 0 | 2 |
| turned on or | (13%) | (4%) | (9%) | (26%) | (15%) | (0%) | (0%) | (15%) |
| excited [symbol] | | | | | | | | |
| Low desire | 3 | 1 | 0 | 4 | 3 | 2 | 1 | 6 |
| | (13%) | (4%) | (0%) | (17%) | (23%) | (15%) | (8%) | (46%) |
| Lack of | 0 | 0 | 3 | 3 | 0 | 0 | 1 | 1 |
| throbbing | (0%) | (0%) | (13%) | (13%) | (0%) | (0%) | (8%) | (8%) |
| Loss of feelings | 0 | 2 | 1 | 3 | — | — | — | — |
| of fullness/ | (0%) | (9%) | (4%) | (13%) | | | | |
| engorgement | | | | | | | | |
| Lack of warmth | 0 | 0 | 2 | 2 | — | — | — | — |
| | (0%) | (0%) | (9%) | (9%) | | | | |
| Discomfort | — | — | — | — | 0 | 1 | 0 | 1 |
| | | | | | (0%) | (8%) | (0%) | (8%) |

*Pulsating was not reported by any participants as the most problematic symptom of FSAD.
**Not all participants listed three symptoms as the most problematic. Some listed/ranked only two. One woman in the postmenopausal group listed the same problem for both number one and number three.
***One postmenopausal woman stated that difficulty achieving an orgasm and low desire were both number one most problematic for her. [symbol]
Due to the way in which premenopausal participants described “turned on” and “excited,” these two concepts were combined as one concept in that group. In the postmenopausal group, which did not report “not feeling excited” as a symptom, this concept is “not feeling turned on.”

### Symptoms Most Important to Treat

Following the question about the most bothersome symptom, participants were asked which symptom would be the most important to have treated, if there was a product on the market to do so. Consistent with the ordering of the most bothersome FSAD symptoms in Table 8, lack of lubrication and wetness was the most desired symptom listed to be treated in the premenopausal group, with seventeen women placing this in their top three (Table 9). Twelve women said loss of feeling/sensation, followed by difficulty or inability to orgasm and lack of tingling (both n=7). These numbers, particularly for the two most important symptoms to treat, match the rankings given by women for those problems that are the most bothersome. Overall, n=22 premenopausal women in total reported wanting to have a treatment for at least one genital arousal sensation issue, including loss of feeling/sensation (n=12), lack of tingling (n=7), and loss of feelings of fullness/engorgement (n=1). This is greater than the number of participants (n=17) reporting lack of lubrication or wetness as the most important FSAD symptom to treat.

In the postmenopausal group, both loss of feeling/sensation and lack of lubrication/wetness were noted as the top two symptoms most desired for treatment (n=9) (Table 9). This was followed by difficulty or inability to orgasm (n=7), and then by low desire (i.e., no interest in sexual activity) (n=5). Not feeling turned on when taking part in sexual activity (cognitive arousal) was listed by n=2 women as a symptom most important to treat, while the specific sensation issues of lack of throbbing and lack of warmth were reported by n=1 woman each. Although the rankings of the most problematic symptoms (Table 8) and the most desired for treatment are slightly different (i.e., difficulty/inability to orgasm is listed as the most problematic and as the third most desired for treatment), results from these two questions suggest that there is congruence between what FSAD symptoms postmenopausal women find bothersome and what they most desire to treat. Overall, n=11 women in total reported wanting to have a treatment for at least one genital arousal sensation issue, including loss of feeling/sensation (n=9), lack of tingling (n=1), and lack of throbbing (n=1). This is greater than the number of participants (n=9) reporting lack of lubrication or wetness as the most important FSAD symptom to treat.

TABLE 9
FSAD Symptoms Pre- and Postmenopausal Participants Most Want to Have
Treated

| Symptoms | | | | | | | | |
|---|---|---|---|---|---|---|---|---|
| Identified as | Premenopausal | | | | Postmenopausal | | | |
| Most Important | #1 | #2 | #3 | Total | #1 | #2 | #3 | Total |
| to Treat* | (n = 23) | (n = 23)** | (n = 23) [symbol] | (N = 23) | (n = 13)*** | (n = 13) | (n = 13) [symbol] | (N = 13) |
| Lack of | 6 | 5 | 6 | 17 | 4 | 1 | 4 | 9 |
| lubrication or | (26%) | (22%) | (26%) | (74%) | (31%) | (8%) | (31%) | (69%) |
| wetness | | | | | | | | |
| Loss of | 5 | 6 | 1 | 12 | 3 | 5 | 1 | 9 |
| feeling/sensation | (22%) | (26%) | (4%) | (52%) | (23%) | (38%) | (8%) | (69%) |
| Difficult or | 4 | 2 | 1 | 7 | 4 | 2 | 1 | 7 |
| inability to | (17%) | (9%) | (4%) | (30%) | (31%) | (15%) | (8%) | (54%) |
| orgasm | | | | | | | | |
| Lack of tingling | 2 | 5 | 0 | 7 | 0 | 0 | 1 | 1 |
| | (9%) | (22%) | (0%) | (30%) | (0%) | (0%) | (8%) | (8%) |
| Not feeling | 2 | 1 | 2 | 5 | 0 | 1 | 1 | 2 |
| turned on or | (9%) | (4%) | (9%) | (22%) | (0%) | (8%) | (8%) | (15%) |
| excited [symbol] | | | | | | | | |
| Low desire | 3 | 2 | 0 | 5 | 3 | 2 | 0 | 5 |
| | (13%) | (9%) | (0%) | (22%) | (23%) | (15%) | (0%) | (38%) |
| Lack of throbbing | 0 | 1 | 2 | 3 | 0 | 0 | 1 | 1 |
| | (0%) | (4%) | (9%) | (13%) | (0%) | (0%) | (8%) | (8%) |
| Loss of feelings | 1 | 1 | 0 | 2 | — | — | — | — |
| of | (4%) | (4%) | (0%) | (9%) | | | | |
| fullness/engorge- | | | | | | | | |
| ment | | | | | | | | |
| Lack of warmth | 0 | 1 | 1 | 2 | 0 | 1 | 0 | 1 |
| | (0%) | (4%) | (4%) | (9%) | (0%) | (8%) | (0%) | (8%) |
| Discomfort | — | — | — | — | 0 | 1 | 1 | 2 |
| | | | | | (0%) | (8%) | (8%) | (15%) |

*Pulsating was not reported by any participants as the most important symptom of FSAD to treat.
**One premenopausal participant listed two symptoms, warmth and tingling, both as the second most important symptom to treat.
***One postmenopausal participant listed two symptoms, orgasm and low desire as the most important to treat. [symbol]
Not all participants listed three symptoms as the most important to treat. Some listed/ranked only two. [symbol]
Due to the way in which premenopausal participants described “turned on” and “excited,” these two concepts were combined as one concept in that group. In the postmenopausal group, which did not report “not feeling excited” as a symptom, this concept is “not feeling turned on.”

### Frequency of FSAD Impacts

When describing experiences with FSAD, participants also discussed impacts that the disorder had on their lives. All of the impacts reported in the premenopausal and postmenopausal cohorts were spontaneous reports by participants. Participants described the impacts of FSAD on their HRQoL in general terms and in some cases described how it relates to specific symptoms they experience. Impacts reported by women fell into five main themes: 1) psychological problems, 2) emotional problems, 3) relationship problems, 4) cognitive problems, and 5) physical problems. The impacts reported with the most frequency in both cohorts were those related to psychological, emotional, and relationship problems.

Decreased confidence was the most reported impact, with nearly all premenopausal (n=21) and postmenopausal (n=12) women reporting this. Participants' descriptions of decreased confidence included discussions of feeling self-conscious, inferior, inadequate, insecure, “not normal,” feeling bad about and for themselves, unattractive, like a failure, invisible, and like “less of a woman.”

In the premenopausal group, the next two most reported impacts were related to relationship problems, namely the effect of FSAD on their partner (n=18) and the difficulties it creates in their relationship (n=16) (Table 10). Participants described the effects of FSAD on the mood of their partner, including feelings of sadness, disappointment, and annoyance. Additionally, participants noted that FSAD can lead to partners questioning themselves, their sexual abilities, and the relationship in general. Following these impacts, the next two most frequently discussed were the emotional problems of frustration and depression, both reported by n=15 women. Over half of the women in this cohort reported experiencing anxiety as an impact of FSAD (n=12). Anger was also described by nearly half of the women in this cohort (n=10).

In the postmenopausal group, the next two most reported impacts were related to emotional problems, namely feeling frustrated and disappointed (n=10 each) (Table 10). Women also noted feeling “disheartened” and “discouraged.” The next most commonly reported impacts, n=9 each, were emotional problems including depression and relationship problems such as difficulties and changes in their relationships, as well as not being interested in sex. Eight postmenopausal women reported that as a result of their FSAD problems they “felt old.” Women (n=7) described feeling bad for their partner, the effects that FSAD has on their partner, an inability to connect with their partner, and feeling angry. Participants' descriptions about the effects of FSAD on their partners included feelings of sadness, disappointment, annoyance, and a general sense that something was lost. Additionally, n=7 women noted that because of the lack of sexual intimacy, it can be difficult to get close to or connect with a partner.

TABLE 10
Frequency of Participant-Reported FSAD Impacts
in Pre- and Postmenopausal Women

| Impact Concept | | Premenopausal | | Postmenopausal | |
|---|---|---|---|---|---|
| Domain | Impact Sub-Concepts | Total (N = 23) | | Total (N = 13) | |
| Psychological | Decreased confidence | 21 | (91%) | 12 | (92%) |
| | Feel bad for partner | 14 | (61%) | 7 | (54%) |
| | Anxiety | 12 | (52%) | 4 | (31%) |
| | Feel old | 7 | (30%) | 8 | (62%) |
| | Guilty | 5 | (22%) | 1 | (8%) |
| | Avoidance of social situations | 3 | (13%) | — | |
| | Feels like a loss of something in life | — | | 6 | (46%) |
| | Feels like body is not working | — | | 5 | (38%) |
| Relationship | Effect on partner | 18 | (78%) | 7 | (54%) |
| | Creates difficulties in the | 16 | (70%) | 9 | (69%) |
| | relationship | | | | |
| | Inability to connect with partner | 8 | (35%) | 7 | (54%) |
| | Not interested in sex | 4 | (17%) | 9 | (69%) |
| | Effect on others (not including | 3 | (13%) | — | |
| | partner) | | | | |
| | Loss of relationship | 2 | (9%) | 1 | (8%) |
| | Sex feels like a chore/work | — | | 4 | (31%) |
| | Cannot find relationship | — | | 4 | (31%) |
| Emotional | Frustrated | 15 | (65%) | 10 | (77%) |
| | Depressed | 15 | (65%) | 9 | (69%) |
| | Angry | 10 | (43%) | 7 | (54%) |
| | Disappointed | 8 | (35%) | 10 | (77%) |
| | Stressed | 5 | (22%) | 2 | (15%) |
| | Distressed | 4 | (17%) | 5 | (38%) |
| | Confused | 4 | (17%) | — | |
| | Less excited about sex | 3 | (13%) | — | |
| | Embarrassed | 2 | (9%) | 3 | (23%) |
| | Jealous of people without FSAD | 1 | (4%) | — | |
| | Helpless | — | | 3 | (23%) |
| | Fearful | — | | 2 | (15%) |
| | Trying to accept the situation | — | | 2 | (15%) |
| | Resentful | — | | 1 | (8%) |
| | Lonely | — | | 1 | (8%) |
| Cognitive | Frequent thinking about FSAD | 7 | (30%) | 2 | (15%) |
| | Mental exhaustion | 2 | (9%) | 1 | (8%) |
| | Blocking FSAD out of mind | 2 | (9%) | — | |
| | Compensating for arousal issues | — | | 1 | (8%) |
| Physical | Soreness in vaginal area | 2 | (9%) | — | |
| | Muscle strengthening | 2 | (9%) | — | |
| | Overall health, physical and mental | 1 | (4%) | — | |
| | Physically tired from trying to have | — | | 1 | (8%) |
| | sex | | | | |

| | | | |
|---|---|---|---|
| Muscle tension | — | 1 | (8%) |
| Weight gain | — | 1 | (8%) |

Women were asked for their preferred term to describe their feelings about their FSAD when given a choice between the words “bothered,” “concerned,” “frustrated,” and “distressed.” The majority of premenopausal (n=19) and postmenopausal (n=7) women chose “frustrated.” Two premenopausal women selected “concerned” and two selected “distressed,” while none chose “bothered.” Four postmenopausal women selected “distressed” and two women selected “bothered,” while none selected “concerned.” Descriptions of the similarities and differences between these words revealed that women see these words as related to one another and on a continuum of intensity with bother being the least intense, concern next, followed by frustration, and then distress.

### Saturation Analysis

For the premenopausal cohort, the results of the saturation analysis based on the reported FSAD symptom concepts demonstrated that only one concept, “pulsating” was introduced in the last set of interviews. All other concepts were introduced in the first set of interviews, and with the exception of throbbing, all were mentioned across all three sets of interviews. Although pulsating was not mentioned until the last set of interviews, it was only mentioned by one participant in this cohort as a symptom. As such, no further interviews were recommended. The saturation analysis also revealed that no participants spontaneously reported “not feeling excited,” but from the descriptions given by participants, this concept appears to be similar to “not feeling turned on.” Saturation was deemed to have been met across the sample for all concepts.

For the postmenopausal cohort, the results of the saturation analysis based on the reported FSAD symptom concepts demonstrated that no concepts were introduced in the last set of interviews. Four concepts, lack of lubrication/wetness, loss of feeling/sensation, difficulty or inability to orgasm, and low desire were spontaneously mentioned by participants in all three sets of interviews. Lack of warmth was not introduced in the first set of interviews but appeared in both the second and third set of interviews. With the exception of discomfort, which was only discussed by participants in the second set of interviews, all other concepts were introduced in the first set of interviews. Given this data, saturation was deemed to have been met. Therefore, because no new concepts emerged in the third set of interviews, additional interviews were not recommended for this sample.

During the interviews, women described a variety of symptoms they experienced related to their FSAD. Premenopausal women described a total of ten symptoms, all of them spontaneously. Eight of these were physical/genital arousal symptoms, and two were related to cognitive arousal/desire. Postmenopausal women described a total of eleven symptoms, eight of them spontaneously. Nine of these were physical/genital arousal symptoms, and two were cognitive arousal/desire related. In contrast to premenopausal women, no postmenopausal women endorsed pulsating, throbbing, or feeling turned on or excited spontaneously. The most frequently reported symptom in both the premenopausal (n=21) and postmenopausal (n=13) cohorts was “inability or difficulty with orgasm.” The high number of reports of difficulty or inability to achieve orgasm are perhaps not surprising given that a healthy genital arousal response (e.g., ability to feel sensations, become wet/lubricated), lacking in this population of women, is a necessary component of the sexual response cycle that precedes orgasm. Many women in both groups referenced that their orgasm problems were specifically due to their lack of genital arousal, demonstrating that orgasmic dysfunction is a secondary impact of FSAD.

The most and second most bothersome symptoms for pre- and postmenopausal women were reversed between groups: premenopausal women said lubrication and wetness was the most problematic symptom (n=17), while postmenopausal said it was loss of feeling/sensation (n=10). The second most problematic symptom for premenopausal women was loss of feeling/sensation (n=12), while it was lubrication and wetness (n=9) for postmenopausal women. Orgasm was the third most problematic symptom for both groups (premenopausal n=8; postmenopausal n=6). The symptom women most desired to have treated was lubrication for premenopausal women, and for postmenopausal women was a tie between lack of lubrication or wetness and loss of feeling and sensation (n=9 for both). Despite the availability of over-the-counter (OTC) lubricants (and reports of women in the postmenopausal group needing to use lubricant to make sex comfortable), women in both groups expressed interest in an FSAD treatment that addresses lack of lubrication. This suggests that OTC lubricants are not addressing the needs of women with FSAD, who reported wanting treatment options to help them produce their own lubrication.

Although “loss of feeling/sensation” was the second most bothersome symptom and second most desired for treatment in premenopausal women and equally desired for treatment along with lubrication and wetness in postmenopausal women, both groups of women in this sample described loss of feeling/sensation in the genitals using a multiplicity of terms [e.g., lack of warmth, lack of tingling, lack of pulsating, lack of throbbing, a lack of throbbing heat, loss of feelings of fullness/engorgement (in both the clitoris and nipples), a lack of feeling or sensation in the genitals, “numbness,” a feeling of “dead” genitals, “lack of blood flow or excitement to the genitals,” and decreased skin sensitivity (includes in nipples and elsewhere on the body)]. These descriptions among both groups of women demonstrate a strong overlap between specific genital arousal-sensation descriptors and the broad “loss of feeling/sensation” term. Additionally, a total of 21 of the 23 premenopausal women and all (n=13) of the postmenopausal women reported a lack of feeling or sensation in the genitals, illustrating “sensation” as the most relevant and important concept for women with FSAD.

Postmenopausal women endorsed an additional physical/genital arousal symptom that did not appear in the premenopausal sample: discomfort. Care was taken by the researchers in this study to separate out those discussions of pain and discomfort as associated with a lack of lubrication and wetness as a side-effect of this symptom from those that were discussed in unrelated terms. Therefore, “discomfort” as described in the postmenopausal sample encompasses those cases described by women as disassociated from lack of lubrication and wetness, encapsulating descriptions of “pain and muscle spasms” and “vaginal tightness that was uncomfortable.” In addition, no postmenopausal women endorsed “not feeling excited” and the only postmenopausal woman who endorsed “not feeling turned on” did so upon probing by the interviewer.

Women in the pre- and postmenopausal cohorts also described a wide variety of ways in which FSAD impacts their HRQoL. The most frequently reported impact in both groups was decreased confidence. In the premenopausal group, the second most reported impact was the effect on the woman's partner and difficulties created in relationships due to FSAD. In the postmenopausal group, this was followed by feelings of frustration, disappointment, and depression, as well as difficulties created in their relationship. Premenopausal women also described a number of emotional impacts, of which the most frequently reported were frustration and depression. Anxiety and anger were also reported frequently among this population. Postmenopausal women also described feeling angry and bad for their partners.

In both the pre- and postmenopausal samples, “frustrated” was offered spontaneously as the term that most describes their feelings about FSAD (n=5 and n=7, respectively). When given a choice between four words (bother, concern, frustrated, distressed), women in both groups again chose frustrated (n=19 and n=7, respectively). Data from both cohorts suggest that participants understood these words as on a continuum, with bother and concern being on one end and frustrated and distressed on the other. Distressed was perceived to be the more “extreme” or “intense” of these words. Women in the postmenopausal group were more willing to endorse or use the word “distressed” than premenopausal women were. This was often linked to the feeling that frustration had gone on for some time and at this point, they felt hopeless or like there would never be a change, and hence were distressed about the situation.

One finding from this study is that women in both cohorts consistently distinguish symptoms of decreased arousal that were unrelated to desire (i.e., they had desire but were unable to transform desire into wanted genital arousal responses). The emphasis on lack of genital sensation and orgasmic dysfunction, and the reports of distress about these issues, illustrate the importance of maintaining arousal as a distinct domain. This calls into question the DSM-5's combination of FSAD and HSDD to form FSIAD, especially given the justification that women are unable to distinguish between desire and arousal. In this study, women mentioned issues of desire in addition to those of arousal but were able to distinguish between the two. Combining desire and arousal into a single diagnostic entity has the potential to exclude women from being treated for these conditions who previously would have met the criteria for either FSAD or HSDD. The symptoms reported in this study support the idea that desire and arousal do not represent the same underlying construct, and therefore the distinct criteria outlined in DSM-IV are more appropriate for diagnosing these conditions. This is aligned with ISSWSH nomenclature which delineates and defines the separate conditions HSDD and FSAD, as well as distinguishes and defines Cognitive and Genital Arousal Disorders. The ISSWSH nomenclature is intended for use by clinicians across disciplines who care for women with FSD.

Results from the study suggest that there is not an appreciable difference in the way that pre- and postmenopausal women experience FSAD. Not only did both groups report similar symptoms, despite a wide age range, they used similar language to do so.

### Conclusions

The analysis of the qualitative data from the interviews revealed a physical and emotional burden of FSAD. The results from the pre- and postmenopausal cohorts suggest that the manifestation of FSAD is similar in these two populations.

### Example 5: Topline Data from the Exploratory Phase 2b Study

Topline results from the study outlined in Example 2 showed Sildenafil Cream-treated patients showed meaningful improvement in the co-primary endpoint assessment that evaluated change from baseline in the Arousal-Sensation Domain of the Sexual Function Questionnaire. Sildenafil Cream-treated patients also showed improvements in the secondary endpoint and pre-specified exploratory endpoints that evaluated sexual satisfaction, arousal lubrication, and achievement and pleasure of orgasm. These data support continued development of Sildenafil Cream and selection of proposed primary endpoint assessments for a Phase 3 study.

The exploratory study was designed to test the sensitivity of several patient reported outcome (PRO) efficacy endpoints and their ability to determine a treatment effect of Sildenafil Cream compared to placebo to inform the ongoing development of Sildenafil Cream. The study also served as a validation study of exploratory endpoints that could be candidate endpoints in a Phase 3 study of Sildenafil Cream. Because there are currently no FDA-approved treatments for FSAD, there are no efficacy endpoints that have been previously validated in a Phase 3 pivotal study for potential treatment for FSAD.

FSAD is the inability to reach or maintain a sufficient physical response to sexual stimulation and, of the various types of female sexual dysfunction disorders, FSAD is most analogous to erectile dysfunction (ED) in men. Sildenafil, a phosphodiesterase-5 (PDE-5) inhibitor, is the active ingredient in a tablet for oral administration currently marketed under the brand name Viagra® for the treatment of ED in men. Sildenafil Cream is an investigational proprietary topical cream formulation of sildenafil specifically designed to increase blood flow to the genital tissue in women. If development is successful, Sildenafil Cream has the potential to be the first FDA-approved treatment for FSAD. Market research suggests that 16% of women in the U.S. ages 21 to 60, or approximately 10 million women, are distressed from experiencing symptoms associated with FSAD, including lack of or low sexual arousal, and are actively seeking solutions to improve their condition. In comparison, the prevalence of complete ED in men is estimated to be about 5% of men at age 40, increasing to about 15% at age 70.

FSAD is distinct from hypoactive sexual desire disorder (HSDD) in women, which is characterized primarily by a lack of sexual desire. FSAD is a condition characterized primarily by a persistent or recurrent inability to attain or maintain sufficient genital arousal (an adequate lubrication-swelling response) during sexual activity, frequently resulting in distress or interpersonal difficulty. As with ED in men, FSAD is associated with insufficient blood flow to the genitalia.

Sildenafil Cream, 3.6% is an investigational proprietary cream formulation of sildenafil, a PDE-5 inhibitor, designed for topical administration to the vulvar-vaginal tissue to increase genital blood flow and provide improvements in the female genital arousal response, while avoiding systemic side effects observed with oral formulations of sildenafil. Sildenafil Cream has been previously evaluated in Phase 1 and Phase 2a clinical studies. In a Phase 1 clinical study in 20 healthy post-menopausal women, topical Sildenafil Cream was safe and well tolerated at clinically relevant doses, and study subjects reported favorable product characteristics: easy to use and readily absorbed. In a Phase 2a study in women with FSAD (15 pre-menopausal and 16 post-menopausal), Sildenafil Cream increased measurable blood flow to the genital tissue compared to placebo cream. Further, data from a thermography study in six healthy women demonstrated significantly greater increases in genital temperature after administration of Sildenafil Cream compared to placebo cream, indicating a positive impact on genital blood flow during the 30-minute testing session, with statistical separation from placebo within the first 15 minutes of dosing.

The exploratory Phase 2b RESPOND study was a multi-center, double-blind, randomized, placebo-controlled study to evaluate the efficacy and safety of Sildenafil Cream in premenopausal patients with FSAD (see Example 2). The study was a first of its kind Phase 2b clinical study that includes PRO instruments to screen eligible women with FSAD and included two co-primary endpoints, a secondary endpoint, and a number of exploratory endpoints to measure, among other things, improvement in localized genital sensations of arousal, reduction in the distress that women experience with FSAD and satisfactory sexual events. There are no FDA-approved treatments for FSAD and thus there are no efficacy endpoints that have been previously approved for use in a Phase 3 pivotal study for potential treatments for FSAD.

The FDA requested that any PROs for use in the assessment of efficacy in a Phase 3 study be adequately validated. Part of that validation includes exit interviews performed as part of the Phase 2b RESPOND study to better understand qualitatively what constitutes a meaningful change to the subjects (see Example 3). These qualitative assessments of meaningful change were utilized to determine which endpoints achieved meaningful improvement in the Sildenafil Cream group.

### Study Summary

The Phase 2b RESPOND study began with 99 subjects in the Sildenafil Cream intent to treat population and 93 subjects in the placebo intent to treat population, with 70 subjects in the Sildenafil Cream intent to treat population and 60 subjects in the placebo intent to treat population completing the study, and therefore was underpowered to assess statistical significance. During the study, participants used Sildenafil Cream and a placebo cream in their home setting and documented their experience using PRO instruments. The study evaluated Sildenafil Cream compared to placebo cream over 12 weeks of dosing following both a non-drug and placebo run-in period.

### Study Efficacy Results and Safety

### Summary of Topline Data

A co-primary endpoint of the Phase 2b RESPOND study was to evaluate the efficacy of Sildenafil Cream compared to placebo as measured by change from baseline to the end of the study in the Arousal-Sensation Domain of the 28-item Sexual Function Questionnaire (SFQ28). The endpoint did not achieve statistical significance. However, the change at the end of the study in the group treated with Sildenafil Cream was consistent with how women reported meaningful improvement in an exit interview at the end of the study.

The other co-primary endpoint was to evaluate the efficacy of Sildenafil Cream compared to placebo as measured by change from baseline to the end of the study in the score for feeling concerned by difficulties with sexual arousal. The change was assessed using the previously validated Female Sexual Distress Scale-Desire, Arousal and Orgasm (FSDS-DAO) Survey. This assessment did not differentiate Sildenafil Cream from placebo.

The secondary endpoint of the study was to evaluate the efficacy of Sildenafil Cream compared to placebo as measured by change from baseline to the end of the study in the number of satisfactory sexual events based on the subjects' response to a question answered and recorded via electronic diary within 24 hours after each sexual event. The endpoint was selected because it can serve as a primary endpoint in a Phase 3 study based on the FDA draft 2016 guidance document for female sexual dysfunction. When measured at the 4- and 8-week mark after randomization, subjects treated with Sildenafil Cream had a higher proportion of satisfying sexual events during the prior month (68.6% and 74.1% at the 4- and 8-week mark, respectively) compared to those treated with placebo (47.9% and 67.9% at the 4- and 8-week mark, respectively) (week 4 p value=0.04).

The exploratory endpoints (selected based on the Content Validity study, see Examples 1 and 4) included endpoints related to genital arousal, genital blood flow, lubrication, orgasm, concerns about difficulties with sexual arousal, and other assessments including questions regarding satisfying sexual events. The exploratory endpoints were assessed at multiple periods over the course of the study via electronic diary.
An exploratory endpoint regarding concerns about difficulties with sexual arousal differentiated Sildenafil Cream versus placebo. Specifically, when asked about their overall impression of change regarding their concerns about difficulties with sexual arousal, women treated with Sildenafil Cream were more likely to report an overall improvement. Based on responses to the question at the 4-, 8- and 12-week mark after randomization, 44% to 49% of the subjects treated with Sildenafil Cream reported an overall improvement compared to 37% to 44% of the subjects treated with placebo (P value<0.01).
Exploratory endpoints related to arousal lubrication and achievement and pleasure of orgasm also demonstrated important differences between the subjects treated with Sildenafil Cream compared to placebo as measured by change from baseline to the 8 week mark after randomization, at P value=0.059 and P value=0.066, respectively (trending toward significance despite the small number of subjects in each group, n=73 in the Sildenafil Cream intent to treat group and n=64 in the placebo intent to treat group for these assessments).
48.7% of the subjects treated with Sildenafil Cream reported an increase in their overall satisfaction with sexual activity measured at the end of the study, compared to 42.6% of the subjects treated with placebo.

Table 11 sets forth certain data regarding the co-primary endpoints, the secondary endpoint, and certain exploratory endpoints. The exploratory Phase 2b RESPOND study was underpowered to assess statistical significance, although statistical significance was achieved on certain assessments.

TABLE 11
Summary of Cited Top Line Data

| | | Sildenafil | Placebo, | P |
|---|---|---|---|---|
| Objective/Endpoint | Measured at | Cream, N⁽¹⁾ | N⁽¹⁾ | value |
| Co-Primary: | End of study | 70 | 60 | >0.05 |
| SFQ28 Arousal | (week 12) | | | |
| Sensation Domain | | | | |
| Co-Primary: | End of study | 70 | 60 | >0.05 |
| FSDS-DAO | (week 12) | | | |
| Survey (Q14 only | | | | |
| (Concerned by | | | | |
| difficulties with | | | | |
| sexual arousal) | | | | |
| Secondary: | Week 4 | 78 | 68 | 0.04 |
| Proportion of | Week 8 | 73 | 64 | >0.05 |
| Satisfactory Sexual | Week 12 | 71 | 61 | >0.05 |
| Events per Month | | | | |
| during Double | | | | |
| Blind Treatment | | | | |
| Period | | | | |
| Exploratory: | Week 8 | 73 | 64 | 0.059 |
| Arousal | | | | |
| Lubrication PROs | | | | |
| Exploratory: | Week 8 | 73 | 64 | 0.066 |
| Orgasm PROs | | | | |
| Exploratory: | Weeks 4-12 | 78 | 68 | <0.01 |
| Regarding Concern | | | | |
| for FSAD | | | | |
| Symptoms⁽²⁾ | | | | |
| Exploratory: | Weeks 4-12 | 78 | 68 | <0.01 |
| Regarding | | | | |
| Satisfactory Sexual | | | | |
| Activity⁽²⁾ | | | | |

⁽¹⁾The numbers in this column represent the number of subjects included in the assessment at the indicated time point.
⁽²⁾This exploratory endpoint was measured periodically beginning at week 4 after randomization through the end of the study.

### Safety

Sildenafil Cream was generally safe and well-tolerated. There were no treatment related serious adverse events and the majority of treatment related adverse events were mild in severity.

### Further Study Results

TABLE 12
Participant Disposition

| Analysis Set | Sildenafil | | Placebo | | Total | |
|---|---|---|---|---|---|---|
| Enrolled in Single-Blind Placebo Run-In Period [n] - | | | | | 252 | |
| visit 3, first day of the 4-week Single-Blind Placebo | | | | | | |
| Run-In Period | | | | | | |
| Screen Failures [n (%)] | | | | | 25 | (9.9) |
| Treated in Single-Blind Placebo Run-In Period [n | | | | | 227 | (90.1) |
| (%)] | | | | | | |
| Randomized to Double-Blind Dosing Period [n] - | 101 | | 99 | | 200 | |
| visit 4, first day of the Double-Blind Dosing Period. | | | | | | |
| Scores at visit 4 are utilized to establish baseline. | | | | | | |
| Treated in Double-Blind Dosing Period [n (%)] | 99 | (98.0) | 94 | (94.9) | 193 | (96.5) |
| Randomized but Not Treated in Double-Blind | 2 | (2.0) | 5 | (5.1) | 7 | (3.5) |
| Dosing Period [n (%)] | | | | | | |
| Completed Treatment [n (%)] | 90 | (89.1) | 84 | (84.8) | 174 | (87.0) |
| Intention to Treat (ITT) Analysis Set [n (%)] | 99 | (98.0) | 93 | (93.9) | 192 | (96.0) |
| Safety Analysis Set (SAS) [n] | 99 | | 134 | | 233 | |
| Per Protocol Analysis Set (PP) [n (%)] | 63 | (62.4) | 54 | (54.5) | 117 | (58.5) |

| | | | |
|---|---|---|---|
| Evaluation of Recall Period Subgroup* (ERS) [n] | 16 | 20 | 36 |

*Approximately n = 30 patients were included for the analyses to investigate whether completion of the Arousal Diary questions influences patient SFQ28 Arousal Sensation domain scores and Question 14 of the FSDS-DAO. These~30 patients completed the entire trial but did not complete the Arousal Diary throughout the trial. These patients were not included in the analysis of the co-primary endpoints.

TABLE 13
Summary of Female Adverse Events

| Adverse Event Category | | | |
|---|---|---|---|
| [Number of Participants | | | |
| (% of Dose Cohort) | Sildenafil | Placebo | Total |
| # Events Reported] | (N = 99) | (N = 94) | (N = 193) |
| Any Treatment Emergent | 29 (29.3) 78 | 28 (29.8) 65 | 57 (29.5) 143 |
| Adverse Event (TEAE) | | | |
| Any Treatment-Related | 14 (14.1) 60 | 14 (14.9) 37 | 28 (14.5) 97 |
| TEAE | | | |
| Any TEAE Leading to | 2 (2.0) 3 | 2 (2.1) 2 | 4 (2.1) 5 |
| Discontinuation of | | | |
| Investigational Product (IP) | | | |
| Any Treatment-Related | 2 (2.0) 3 | 0 | 2 (1.0) 3 |
| TEAE Leading to | | | |
| Discontinuation of IP | | | |

TABLE 14
Adverse Events (Most Common Female TEAEs)

| | Sildenafil | | Placebo | |
|---|---|---|---|---|
| ADVERSE EVENTS | (N = 99) | | (N = 94) | |
| Any Adverse Events | 29 | (29.3) | 28 | (29.8) |
| Infections and Infestations | 16 | (16.2) | 16 | (17.0) |
| (N Reporting at Least 1 | | | | |
| TEAE, % Group) | | | | |

| | | |
|---|---|---|
| COVID 19 Infection | 5 | 4 |
| Bacterial Vaginosis | 1 | 2 |
| Vulvovaginal Candidiasis | 2 | 1 |
| Urinary Tract Infection | 0 | 1 |
| Other Infections (GI, | 8 | 11 |
| Respiratory, Sinusitis etc.) | | |

| | | | | |
|---|---|---|---|---|
| Administrator Site (N | 12 | (12.1) | 14 | (14.9) |
| Reporting at Least 1 | | | | |
| TEAE, % Group) | | | | |

| | | |
|---|---|---|
| Application Site Discomfort | 12 | 14 |
| Application Site Erythema | 3 | 3 |
| Application Site Edema | 2 | 1 |
| Vulvovaginal Erythema | 1 | 0 |

| | | | | |
|---|---|---|---|---|
| Reproductive System and | 1 | (1.0) | 5 | (5.3) |
| Breast Disorders (N Reporting | | | | |
| at Least 1 TEAE, % Group) | | | | |

| | | |
|---|---|---|
| Vulvovaginal Discomfort | 0 | 2 |
| Vulvovaginal Erythema | 1 | 0 |
| Cervical friability, | 0 | 3 |
| Dysmenorrhea, Dyspareunia | | |

TABLE 15
Number of Sex Acts with the Investigational
Product during double-blind dosing period

| Sildenafil | Placebo | Total |
|---|---|---|
| (N = 99) | (N = 94) | (N = 193) |
| 1131 | 951 | 2082 |

TABLE 16
Summary of Male Partner TEAEs

| Adverse Event Category | | | |
|---|---|---|---|
| [Number of Participants | | | |
| (% of Dose Cohort) | Sildenafil | Placebo | Total |
| # Events Reported] | (N = 91) | (N = 84) | (N = 175) |
| Any TEAE | 7 (7.7) 9 | 4 (4.8) 4 | 11 (6.3) 13 |
| Any Treatment-Related TEAE | 3 (3.3) 5 | 0 | 3 (1.7) 5 |
| Any TEAE Leading to | 1 (1.1) 1 | 0 | 1 (0.6) 1 |
| Discontinuation of IP | | | |
| Any Treatment-Related TEAE | 1 (1.1) 1 | 0 | 1 (0.6) 1 |
| Leading to Discontinuation of IP | | | |

TABLE 17
Most Common Male Partner TEAEs

| | | |
|---|---|---|
| ADVERSE EVENTS - | | |
| Summary of most | Sildenafil | Placebo |
| common male partner TEAE | (N = 91) | (N = 84) |

| | | | | |
|---|---|---|---|---|
| Any Adverse Events | 7 | (7.7) | 4 | (4.8) |
| Infections and Infestations (N Reporting | 3 | (3.3) | 3 | (3.6) |
| at Least 1 TEAE, % Group) | | | | |

| | | |
|---|---|---|
| COVID 19 Infection | 3 | 2 |
| Sinusitis | 0 | 1 |

| | | | | |
|---|---|---|---|---|
| Administrator Site (N Reporting | 1 | (1.1) | 0 | (0) |
| at Least 1 TEAE, % Group) | | | | |

| | | |
|---|---|---|
| Application Site Discomfort | 1 | 0 |

| | | | | |
|---|---|---|---|---|
| Reproductive System (N Reporting | 1 | (1.1) | 0 | (0) |
| at Least 1 TEAE, % Group) | | | | |

| | | |
|---|---|---|
| Genital Hypoaesthesia | 1 | 0 |

TABLE 18
Exploratory Endpoint: SFQ28 Desire Domain

| | Sildenafil | | | | | Placebo | | | | | LS | | B |
|---|---|---|---|---|---|---|---|---|---|---|---|---|---|
| Week | | | | | Mean | | | | | Mean | Diff | | 80% |
| SFQ28 | | | | | CHG | | | | | CHG | Mean | | α |
| Desire | N | Mean | SD | Medium | BL | N | Mean | SD | Medium | BL | (SE) | P | 0.05 |
| BL | 84 | 13.8 | 3.70 | 14.0 | NA | 72 | 14.3 | 3.68 | 15.0 | NA | NA | NA | NA |
| Week | 78 | 14.4 | 3.53 | 14.0 | 0.5 | 69 | 14.8 | 4.09 | 15.0 | 0.6 | −0.2 | 0.726 | 1643 |
| 4 | | | | | (2.61) | | | | | (3.20) | (0.45) | | |
| Week | 73 | 14.6 | 4.09 | 15.0 | 1.0 | 64 | 13.9 | 4.37 | 15.0 | −0.3 | 1.2 | 0.022 | 537 |
| 8 | | | | | (2.81) | | | | | (3.27) | (0.51) | | ** |
| Week | 70 | 14.5 | 4.30 | 14.5 | 0.9 | 60 | 14.5 | 3.97 | 15.0 | 0.2 | 0.7 | 0.218 | 29027 |
| 12 | | | | | (3.46) | | | | | (3.78) | (0.59) | | |

The data in Table 18 were determined based on the SFQ Desire Domain (Range 5-31):
(Q1) How often have you had pleasurable thoughts and feelings about sexual activity? (Q
(Q2) How often have you wanted to be sensually touched and caressed by your partner?
(Q3) How often have you wanted to take part in sexual activity?
(Q4) How often have you initiated sexual activity with your partner?
(Q14) How often did you take part in sexual activity with penetration (e.g., vaginal penetration and intercourse)?
(Q26) How often did you look forward to sexual activity?

Sildenafil Cream-treated patients showed meaningful improvement in the co-primary endpoint assessment that evaluated change from baseline in the Arousal-Sensation Domain of the Sexual Function Questionnaire, although the endpoint did not achieve statistical significance. Sildenafil Cream-treated patients also showed improvements in the secondary endpoint and pre-specified exploratory endpoints that evaluated various aspects of the sexual experience, including sexual satisfaction, arousal lubrication, achievement and pleasure of orgasm, and sexual desire.

### SUMMARY

### 1. More Satisfying Sexual Events

Women treated with Sildenafil Cream had a greater proportion of satisfying sexual events.

68.6% and 74.1% at the 4- and 8-week mark, respectively for Sildenafil Cream compared to those treated with placebo (47.9% and 67.9% at the 4- and 8-week mark, respectively) (week 4 P value=0.04).

2 Improvement in Overall Impression of Change Regarding their Concerns about Difficulties with Sexual Arousal,

Women treated with Sildenafil Cream were more likely to report an overall improvement.

44% to 49% of the subjects treated with Sildenafil Cream reported an overall improvement compared to 37% to 44% of the subjects treated with placebo based on responses to the question at the 4-, 8- and 12-week mark after randomization (P value<0.01).

### 3 Improvement in Arousal Lubrication

Women treated with Sildenafil Cream reported greater change from baseline to the 8-week mark after randomization compared to placebo (P value=0.059)

### 4. Improvement in Achievement and Pleasure of Orgasm

Women treated with Sildenafil Cream reported greater change from baseline to the 8-week mark after randomization compared to placebo (P value=0.066)

### 5. Improvement in Sexual Desire

Women treated with Sildenafil Cream reported greater change from baseline to the 8-week mark after randomization compared to placebo (P value=0.022)

Response noted in domains of physical arousal and interest; improvement in multiple facets of sexual dysfunction:
Arousal lubrication
Orgasm
Desire

Maximum separation from placebo was demonstrated within 8 weeks post randomization. For those assessments where improvement was noted following use of Sildenafil Cream, the improvement was achieved by week 8 and maintained through the end of study (week 12), demonstrating persistence while on treatment, whereas, in the placebo group, no such trends were observed with no such improvements achieved at the week 8 mark.

Concordance between arousal diary and 28-day recall provides support for endpoints that reduce patient burden. For purposes of correlation comparison of the 24-hr recall Arousal Diary and 28-day recall, a number of patients (n=30) were enrolled and randomized into a Recall Period Evaluation sub-group. The patients in the Recall Period Evaluation sub-group participated in the entire study but did not complete the Arousal Diary. Arousal Diary and 28-day recall responses were similar between groups, providing support for the less burdensome longer recall period.

### Additional Results

Further analyses of Phase 2b RESPOND study data identified a subset of study participants-women with FSAD as well as women with FSIAD whose primary complaint was arousal dysfunction (reduced response to physical sexual stimulation)—that achieved statistically significant and clinically meaningful improvement in the Phase 2b co-primary endpoint of evaluating the efficacy of Sildenafil Cream versus placebo cream as measured by change from baseline to the end of the 12-week double-blind dosing period in the Arousal-Sensation Domain of the Sexual Function Questionnaire (p=0.04). In addition, only the Sildenafil Cream treatment group in this subset, and not the placebo group, demonstrated clinically meaningful improvement at the end of the 12-week period.

The other co-primary objective of the Phase 2b RESPOND study was to evaluate the efficacy of Sildenafil Cream compared to placebo cream as measured by the change from baseline to the end of the 12-week double-blind dosing period in the score for feeling concerned by difficulties with sexual arousal utilizing the Female Sexual Distress Scale-Desire, Arousal, Orgasm (FSDS-DAO). Analyses of the same subset of women in the Phase 2b RESPOND study (women with FSAD and women with FSIAD whose primary complaint was arousal dysfunction) demonstrated that the Sildenafil Cream treatment group achieved statistically significant improvement in several FSDS-DAO questions, including regarding guilt, stress, inadequacy, and embarrassment due to their sexual problems (p=0.02 to 0.05).

### Example 6: Assessment of Psychometric Properties

Psychometric properties refer to the validity and reliability of the questionnaire measurement tools. The psychometric validation focused on evaluating the reliability, validity, and responsiveness of these measures and establishing estimates of meaningful change for select item/domain/total scores.

In this study, the SFQ28, FSDS-DAO, and Arousal Diary are among the PRO measures to assess outcomes for the FSAD patients (Primary: SFQ28 Arousal Sensation and FSDS-DAO Item 14; Secondary: Arousal Diary Item 12; Exploratory: other select scores from the 3 PRO instruments).

An assessment of psychometric properties as well as estimating meaningful change for the SFQ28, FSDS-DAO, and Arousal Diary was performed to support associated labeling claims in women with FSAD.

This assessment discloses the item-level performances and domain scoring algorithm of the SFQ28, FSDS-DAO, and Arousal Diary in women with FSAD.

Psychometric analyses was conducted on the patient reported outcome population, which is a subset of the Full Analysis Set population and included all patients who completed the FSDS-DAO and at least 1 SFQ28 items at Screening (Visit 1).

### Results

### SFQ28

### Stage 1: Quality of Completion and Individual Item Performance

The SFQ28 had good quality of completion, with over 90% completion rates across relevant Visits 1, 3, 4, and 7. Only a few participants did not respond to the instrument, with missing rates ranging from 0%-6.3% across visits. Ceiling and floor effects were observed for 6 and 7 items, respectively, at Baseline (Visit 4). Ceiling effects were observed mostly in the Pain and Partner domains. Only 2 pairs of items (Items 18 and 19, and Items 18 and 25) in the Enjoyment domain had low correlations (<0.20) with one another, and up to 8 pairs of items across 6 domains (Desire, Arousal Sensation, Arousal Lubrication, Arousal Cognitive, Pain, and Orgasm) correlated very highly at >0.80, indicating good inter-item correlations but could also be considered for redundancy. Two item pairs in the Desire domain were highly correlated:
Item 1—How Often Pleasurable Thoughts About Sex/Item 3-How Often Wanted to Take Part in Sex (0.817);
Item 2—How Often Want to be Touched by Partner/Item 3-How Often Wanted to Take Part in Sex (0.817).

Two item pairs in the Arousal Sensation domain were highly correlated:
Item 6—How Often Feeling of Warmth in Vagina/Item 7-How Much Warmth in Vagina (0.959);
Item 8—How Often Sensation of Pulsating/Item 9—How Much Pulsating/Tingling in Vagina (0.891).

Item-domain correlations were also acceptable, with only 2 items (Item 18 and Item 25) in the Enjoyment domain with <0.40 correlations with the corrected domain score (both corrected correlations≥0.39). Confirmatory factor analysis (CFA) was conducted to assess the dimensional structure of the SFQ28, and the results supported the 8-factor model proposed by the scale developers. Four out of 5 model fit indices met the acceptability benchmarks: comparative fit index (CFI)>0.90, non-normed fit index (NNFI)>0.90, root mean square error of approximation (RMSEA)<0.08, and standardized root mean squared residual (SRMR)<0.08. In terms of the relationship between an item and its corresponding factor, 21 out of 28 items achieved a standardized loading factor>0.60. The 6 items with factor loadings<0.60 were the following: Item 14 (Desire domain), Items 8 and 9 (Arousal Sensation domain), and Items 18, 19, 21, and 25 (Enjoyment domain).

The SFQ28 had high completion rates and individual item performance. None of the SFQ28 items were removed and the proposed factor model was retained to inform the scoring of domains to use in Stage 2 and Stage 3.

### Stage 2: Domain-Level Reliability and Validity

Five SFQ28 domains (Desire, Arousal Sensation, Orgasm, Pain, and Enjoyment domains) with at least 3 items per domain were subject to internal consistency reliability analysis. All 5 domains demonstrated strong internal consistency ranging from 0.710 to 0.845.

Test-retest reliability of the 8 SFQ28 domains was examined using the stable subpopulation on the Patient Global Impression of Severity (PGI-S) from Visit 1 to Visit 3, as well as the stable subpopulation on the Patient Global Impression of Change (PGI-C) from Visit 3 to Visit 4. The Desire domain (intraclass correlation coefficient [ICC]=0.731) demonstrated acceptable test-retest reliability from Visit 1 to Visit 3 when using the PGI-S. Between Visit 3 and Visit 4, ICCs for 4 domains (Desire, Arousal Cognitive, Pain, and Partner) when using the PGI-C exceeded 0.70, ranging from 0.730 to 0.822.

Correlations between the SFQ28 domains and scores on two reference measures, including the Arousal Diary and the FSDS-DAO, were calculated to assess convergent validity of the SFQ28. Overall, the SFQ28 demonstrated acceptable convergent validity. The 7 domains exhibited moderate (>0.30) or high (>0.50) correlations with at least one score on the reference measures. Strongest positive correlations were between the Arousal Sensation, Arousal Cognitive, and Orgasm domain scores on the SFQ28 and scores on the Arousal Diary (Arousal Sensation, Genital Arousal, and Genital Arousal Concerns domains, and Item 12-Number and Proportion of Satisfactory Sexual Events [SSEs]). The Arousal Sensation domain had correlations with all reference measures on the Arousal Diary. The Desire domain had a correlation with the Genital Arousal domain of the Arousal Diary.

Divergent validity of the SFQ28 was assessed using the GAD-7 and PHQ-8 as reference measures. Correlations between all SFQ28 domains and the GAD-7 and PHQ-8 were very small (maximum correlation<0.13), demonstrating good divergent validity of the SFQ28.

To evaluate known-groups validity of the SFQ28, the PGI-S (4 response categories: None, Mild, Moderate, Severe) and Patient Global Impression of Frequency (PGI-F) (5 response categories: Not at all, Sometimes, Often, Very often, Every time) were used to define known groups. In general, the SFQ28 trended downward with increasing severity and frequency of genital arousal problems. Moderate to large effect sizes (≥0.5) were found mostly in the comparisons between groups that reported Moderate (on the Arousal Sensation domain) and Severe genital arousal problems, and between groups who reported experiencing genital arousal problems Very often and Every time for 6 out of 8 domains (the exceptions were Arousal Lubrication and Partner domains).

To evaluate responsiveness, change scores on the reference measures (PGI-S, PGI-F, and PGI-C) from Visit 4 to Visit 7 were stratified into 4 levels: Marked Improvement, Minimal Improvement, Stable, and Worsening. The responsiveness of 6 domain scores of the SFQ28 were examined: the Arousal Sensation, Arousal Lubrication, Arousal Cognitive, Desire, Orgasm, and Pain domains. Comparisons of change scores on these SFQ28 domains between adjacent groups of reference score change revealed that these domains were most responsive among patients who experienced sexual functioning improvement (i.e., distinguished patients with marked improvement versus minimal improvement well), and less responsive among patients whose condition remained stable or worsened.

Overall, Stage 2 analytical results supported the internal consistency reliability, convergent and divergent validity, known-groups validity, and responsiveness of SFQ28.

### Stage 3: Estimation of Meaningful Change Threshold

Five SFQ28 domains (Arousal Cognitive, Arousal Lubrication, Arousal Sensation, Desire, and Orgasm) correlated with at least 1 anchor at ≥0.35. Distribution-based results were used as supporting information to derive the meaningful within patient change (MWPC). Only distribution-based methods were used to obtain the MWPC estimate for the Pain domain.

Change scores from Visit 4 to Visit 7 on all anchors were stratified into 4 categories: Marked Improvement, Minimal Improvement, Stable, and Worsening. The process for arriving at the final recommendation is described for the Arousal (Sensation) domain of SFQ28, and the same method was followed for other domain or total scores. For example, for the Arousal (Sensation), the recommended MWPC is 1.5. This is based on the fact that 1.5 falls outside the CI for the no change category and within the CI for a minimal improvement (1-category improvement), and just below the lower bound of the CI for the marked improvement (2 or 3 category improvement) on the PGI-S anchor with a large SES of 0.940 (Error! Reference source not found.9). Results from the meta-analytic approach (forest plot) which weights the mean change by sample size and within-group variability also supports the proposed 1.5 point change as the MWPC, as it is close to the mean of the overall Minimal improvement group and does not fall within the 95% CI of the adjacent groups.

TABLE 19
Within-Groups Collapsed Anchor-Based Meaningful Change Derivation for the
SFQ28 Arousal (Sensation) at Visit 7, Using the PGI-S as an Anchor; Population: PRP
(N = 192)

| | | | | Mean | | | | | |
|---|---|---|---|---|---|---|---|---|---|
| | Change | Correlation | | Change | | Min, | 95% | P | |
| Change in Anchor | Correlation^{[1]} | at Visit 4^{[1]} | n | (SD) | Median | Max | CI | value^{[2]} | SES^{[3]} |
| Marked | −0.398 | −0.454 | 12 | 4.58 | 4.50 | −2.00, | 1.561, | 0.0066 | 1.656 |
| improvement: −3 to | | | | (4.757) | | 12.00 | 7.606 | | |
| −2 | | | | | | | | | |
| Minimal | | | 35 | 2.60 | 2.00 | −4.00, | 1.421, | <.0001 | 0.940 |
| improvement: −1 | | | | (3.432) | | 9.00 | 3.779 | | |
| Stable: 0 | | | 94 | 0.34 | 0.00 | −6.00, | −0.292, | 0.2880 | 0.123 |
| | | | | (3.088) | | 15.00 | 0.973 | | |
| Worsened: +1 to | | | 20 | −0.15 | 0.00 | −4.00, | −1.216, | 0.7715 | 0.054 |
| +3 | | | | (2.277) | | 5.00 | 0.916 | | |

CI = confidence interval; PGI-S = Patient Global Impression of Severity; PRP = patient-reported outcome population; SD = standard deviation; SES = standardized effect size; SFQ28 = Sexual Function Questionnaire
^{[1]}Polyserial/polychoric correlations were calculated for Visit 4 and polyserial correlations were calculated for change from Visit 4.
^{[2]}The P value for each individual change group is derived from a paired (within samples) t-test assessing the difference over time.
^{[3]} Standardized effect sizes are calculated as the mean change divided by the standard deviation at Baseline. They are judged as: small = 0.20, moderate = 0.50, and large = 0.80.

By applying this process to determine the MWPC for other domains, this led to an estimation of at least a 1-point change as the MWPC. Most recommended MWPCs were within the 95% CI for the Minimal Improvement category, outside of the 95% CI for the Stable group and just below the Marked Improvement CI lower bound, with moderate or large SES. Cumulative distribution function and probability distribution function curves illustrated the separation between each change category on the anchor and provided graphical evidence to support the recommended MWPC derived from the anchor-based method. The recommended MWPCs were also generally larger than 1 standard error of measurement and 0.5 standard deviation. A summary of the results for reliability, validity, and recommended MWPCs for the SFQ28 is presented in Table 20 below.

TABLE 10
Summary of Stage 2 and Stage 3 for the SFQ28

| | | Test- | | | Known- | | |
|---|---|---|---|---|---|---|---|
| SFQ28 | Internal | retest | Convergent | Divergent | groups | | MWPC |
| domains | Consistency | Reliability | Validity | Validity | Validity | Responsiveness | Estimate |
| Arousal | | ✓ | Moderate | Strong | ✓ | ✓ | 1.00 |
| (C) | | | overall | | | | |
| Arousal | | X | Weak | Strong | X | ✓ | 1.00 |
| (L) | | | overall | | | | |
| Arousal | ✓ | X | Moderate to | Strong | ✓ | ✓ | 1.50 |
| (S) | | | Strong | | | | |
| | | | overall | | | | |
| Desire | ✓ | ✓ | Weak | Strong | ✓ | ✓ | 2.00 |
| | | | overall | | | | |
| Enjoyment | ✓ | X | Weak to | Strong | ✓ | | |
| | | | Moderate | | | | |
| | | | overall | | | | |
| Orgasm | ✓ | X | Moderate | Strong | ✓ | ✓ | 1.20 |
| | | | overall | | | | |
| Pain | ✓ | ✓ | Weak | Strong | ✓ | ✓ | 1.00 |
| | | | overall | | | | |
| Partner | | ✓ | Weak | Strong | X | | |
| | | | overall | | | | |

Arousal (C) = Arousal Cognitive, Arousal (L) = Arousal Lubrication, Arousal (S) = Arousal Sensation
✓ = exceed acceptability threshold, X = below acceptability threshold
Blank cells indicate the corresponding measurement properties were not examined for the domain.

### FSDS-DAO

### Stage 1: Quality of Completion and Individual Item Performance

The FSDS-DAO had good quality of completion, with over 90% completion rates across Visits 1, 3, 4, and 7. A ceiling effect was observed in 2 items, and a floor effect was observed in 5 items. Item pairs with low correlations were the following: Item 15-Felt Frustrated by Problems With Orgasm had low correlations (<0.20) with 8 items
Item 1—How Often Distressed About Sex Life (0.159);
Item 2—Unhappy About Sexual Relationship (0.073);
Item 5—How Often Stressed About Sex (0.118);
Item 7—How Often Felt Worried About Sex (0.095);
Item 9—How Often Felt Regrets About Sexuality (0.127);
Item 10—Felt Embarrassed About Sexual Problems (0.136);
Item 11—How Often Felt Dissatisfied w/Sex Life (0.198);
Item 13—How Often Bothered by Low Sexual Desire (0.029);
Item 13—How Often Bothered by Low Sexual Desire had a low correlation with Item 4-Felt Frustrated by Sexual Problems (0.174).

However, none of the item pairs were highly correlated (>0.80), indicating acceptable inter-item correlations. There were 2 items in the FSDS-DAO wherein the correlation with the total score was <0.40:
Item 13 (How Often Bothered by Low Sexual Desire)
Item 15 (Felt Frustrated by Problems with Orgasm)

A CFA was fitted to the FSDS-DAO data to test for a unidimensional model. Fit statistics showed CFI, NNFI, and SRMR exceeded the acceptability benchmarks (CFI>0.90, NNFI>0.90, SRMR<0.08), but the χ² test and RMSEA did not (χ² was significant, P<0.05; RMSEA>0.10). Most items in the FSDS-DAO reached the factor loading threshold of 0.60.

Of note, Item 5 was among the best performing items with no floor or ceiling effects, acceptable correlations with 13 out of 14 remaining items (except Item 15), highest correlation with the FSDS-Total (0.742), and strongest association with the FSDS-DAO factor (standardized factor loading at 0.805).

### Stage 2: Domain-Level Reliability and Validity

The FSDS-DAO had an overall internal consistency of 0.903. The test-retest reliability of the FSDS-DAO was assessed using the stable Patient Global Impression of Change in Concern with Sexual Difficulties (PGI-C Concern) subpopulation from Visit 3 to Visit 4, and the ICC for the FSDS-DAO was 0.710, meeting the threshold for test-retest reliability. For the evaluation of convergent and divergent validity, all correlations with the Arousal Diary and SFQ28 were negative correlations (−0.574 to −0.080. In terms of known-groups validity, moderate effect sizes (>0.5) were observed when comparing “Moderate” versus “Severe”, “Mild” versus “Severe” PGI-S categories, as well as when comparing “Sometimes” versus “Often” PGI-F categories. The FSDS-DAO was most responsive (between-group SRM>0.5) among patients who experienced improvement in sexual dysfunction and was particularly able to distinguish those with minimal versus marked improvement.

### Stage 3: Estimation of Meaningful Change Threshold

The same process to select the MWPC as described for the SFQ28 was applied to the FSDS-DAO. The recommended MWPC for the FSDS-DAO Total Score improvement was-7 points. A summary of the results for reliability, validity, and recommended MWPC thresholds for the FSDS-DAO Total is presented in Table 21 below.

TABLE 21
Summary of Stage 2 and Stage 3 for the FSDS-DAO Total

| Internal | Test-retest | Convergent | Divergent | Known-groups | | MWPC |
|---|---|---|---|---|---|---|
| Consistency | Reliability | Validity | Validity | Validity | Responsiveness | Estimate |
| ✓ | ✓ | Weak | Strong | ✓ | ✓ | −7.0 |

✓ = exceed acceptability threshold, X = below acceptability threshold

### Arousal Diary

### Stage 1: Quality of Completion and Individual Item Performance

The Arousal Diary had good completion rates of >90% among participants who were expected to complete this measure (non-members of the Recall Period Evaluation subgroup) across Visits 3, 4, and 7. Four items (Items 9-12) exhibited a ceiling effect and a floor effect was observed for 5 items (Items 2-5, and Item 12). Inter-item correlations were good, with Item 7 and Item 8 being the only item pair with a correlation>0.80 and there was no item pair with a correlation<0.20. All item-domain correlations were also good (>0.40).

An exploratory factor analysis (EFA) was conducted on Items 1-10 of the Arousal Diary, with Parallel Analysis (PA), minimum average partial (MAP), and user-defined factor extraction used to guide factor retention. PA suggested 1 factor underlying responses to Items 1-10, whereas MAP recommended 2 factors. The user-determined extraction showed 3 factors with the following item grouping: Factor 1: Items 2-5; Factor 2: Items 8-10; Factor 3: Items 1, 6 and 7. The 3-factor structure was retained because of its theoretical alignment. The 3 domains were labelled as Arousal Sensation (Items 2-5), Genital Arousal (Items 1, 6, and 7), and Genital Arousal Concerns (Items 8-10). These domains were used in Stage 2 and Stage 3.

### Stage 2: Domain-Level Reliability and Validity

The Arousal Diary Genital Arousal domain, Arousal Sensation domain, and Genital Arousal Concerns domain had an overall Cronbach's a, measuring internal consistency, of 0.830, 0.903, and 0.907, respectively, indicating high internal consistency. The test-retest reliability of the Genital Arousal domain, using the PGI-S subpopulation from Visit 3 to Visit 4, resulted in an ICC of 0.710, meeting the threshold for test-retest reliability.

For the evaluation of convergent and divergent validity, correlations with the SFQ28 were positive correlations, and mostly ranged from small to moderate. One notable exception was the Arousal Sensation domain on the Diary and the Arousal Sensation domain on the SFQ28 (0.785). Arousal Sensation, Genital Arousal, and Genital Arousal Concerns had at least a moderate correlation with the SFQ28 Arousal Cognitive, Arousal Sensation, and Orgasm domains, demonstrating acceptable convergent validity. For the Arousal Diary Item 12, both number and proportion of SSEs also had moderate correlation with the SFQ28 Arousal Sensation and Orgasm domains. Only the Genital Arousal Concerns domain from the Diary moderately correlated with the FSDS-DAO Total score with a negative correlation, which further evidenced its acceptable convergent validity.

For known-groups validity, the Arousal Diary was able to discriminate participants of different levels of genital arousal problem severity, especially among those who reported at least mildly severe problems on the PGI-S. Evidence for known-groups validity among participants with different frequencies of genital arousal problems showed moderate effect size in comparisons involving mostly participants who experienced genital arousal problems with high frequency (Very often and Every time) when evaluated using the PGI-F.

All Arousal Diary domains demonstrated adequate responsiveness in that they were able to discriminate patients whose severity of genital arousal problems minimally improved versus remained stable, and those who reported stable versus worsened when using the PGI-S as an anchor. Furthermore, all domain scores and Item 12 (Proportions and Number of SSEs) were responsive among patients who reported minimal versus marked improvement, and stable versus worsened arousal problems when using the PGI-C.

### Stage 3: Estimation of Meaningful Change Threshold

The same process to select the MWPC as described for the SFQ28 was applied to the Arousal Diary. The recommended MWPCs for the Arousal Sensation, Genital Arousal, and Genital Arousal Concerns were 1.45, 1.13, and 1.49, respectively. The recommended MWPC for Item 12-Proportion of Satisfactory Sexual Events was 0.25. A summary of the results for reliability, validity, and recommended MWPCs for the Arousal Diary is presented in Table 22.

TABLE 22
Summary of Stage 2 and Stage 3 for the Arousal Diary

| | | | | | Known- | | |
|---|---|---|---|---|---|---|---|
| Arousal Diary | Internal | Test-retest | Convergent | Divergent | groups | | MWPC |
| domains/items | Consistency | Reliability | Validity | Validity | Validity | Responsiveness | Estimate |
| Arousal (S) | ✓ | X | Moderate to | Strong | ✓ | ✓ | 1.45 |
| | | | strong | | | | |
| GA | ✓ | ✓ | Moderate | Strong | ✓ | ✓ | 1.13 |
| GA Concerns | ✓ | X | Moderate | Strong | ✓ | ✓ | 1.49 |
| Item 12 - | | X | Weak to | Strong | ✓ | ✓ | 1.41* |
| Number of | | | Moderate | | | | |
| SSEs | | | | | | | |
| Item 12- | | X | Weak to | Strong | ✓ | ✓ | 0.25 |
| Proportion of | | | Moderate | | | | |
| SSEs | | | | | | | |

Arousal (S) = Arousal Sensation, GA = Genital Arousal, GA Concerns = Genital Arousal Concerns, SSE = Satisfactory Sexual Event
✓ = acceptable; X = below acceptability threshold; * = distribution-based estimate.
Blank cells indicate the corresponding measurement properties were not examined for the domain/item.

### Conclusion

The SFQ28 demonstrated, in general, acceptable item performance, internal consistency, convergent and divergent validity, known-groups validity, and responsiveness.

In particular, the Arousal Sensation domain had strong internal consistency, moderate to high convergent validity, strong divergent validity, large standardized-effect sizes indicating strong known-groups validity, and was shown to be responsive. The Arousal Sensation domain had two item pairs with high correlations indicating a strong relationship. The Desire domain had strong internal consistency, and acceptable test-retest changes, strong divergent validity, moderate standardized effect sizes indicating acceptable known-groups validity, and was shown to be responsive. The Desire domain had two item pairs with high correlations suggesting a strong relationship.

The FSDS-DAO demonstrated acceptable item performance, internal consistency, test-retest validity, divergent validity, known-groups validity, and responsiveness. Item 5 did not demonstrate floor or ceiling effects, and had adequate correlation with 13 out of 14 remaining items within the FSDS-DAO instrument, and strong relationships with the FSDS-DAO Total score as well as the FSDS-DAO factor.

Lastly, the Arousal Diary demonstrated acceptable item performance, internal consistency, convergent and divergent validity, known-groups validity, and responsiveness.

The MWPCs derived for the SFQ28, FSDS-DAO Total Score, and the Arousal Diary can enhance the interpretation of these outcomes for evaluating meaningful benefit in patients with FSAD.

The overall results from this psychometric validation can inform the endpoint strategy of the future phase 3 evaluation of Sildenafil Cream, 3.6% for treating FSAD. In particular, the strong results regarding the SFQ28 Arousal Sensation domain together with positive test-retest reliability findings for the SFQ28 from previous research provide support for the continued use of this domain as a primary endpoint for phase 3. Similarly, Item 5 is among the items with the strongest psychometric performance in the FSDS-DAO and is recommended as an appropriate measure of sexual stress for endpoint derivation in phase 3 trials. Psychometric Analysis Summary is shown in Table 23.

TABLE 23
Analysis Summary

| | | Meaningful Within- |
|---|---|---|
| | | Patient Change (MWPC) |
| Instruments | Domains/Items | Threshold Estimates |
| SFQ28 | Arousal (Cognitive) | 1 |
| | Arousal (Lubrication) | 1 |
| | Arousal (Sensation) | 1.5 |
| | Desire | 2 |
| | Orgasm | 1.20 |
| | Pain | 1 (distribution-based |
| | | ½SD = 0.98, SEM = 0.85) |
| FSDS-DAO | Total Score | −7 |
| Arousal | Arousal Sensation | 1.45 |
| Diary | Genital Arousal | 1.13 |
| | Genital Arousal Concerns | 1.49 |
| | Item 12 - Proportion of SSEs | 0.25 |
| | Item 12 - Number of SSEs | 1.41 (distribution- |
| | | based ½SD = 1.40) |

### Example 7: Sexual Experiences in an Exploratory, Phase 2b, Randomized, Double-Blind, Placebo-Controlled Clinical Trial of Sildenafil, 3.6% Cream for the Treatment of Female Sexual Arousal Disorder

Quantifying the change in the mean number and mean proportion of satisfactory sexual experiences (SSEs) are efficacy endpoints recommended by the United States Food and Drug Administration in studies of treatments for female sexual dysfunction (FSD), including female sexual arousal disorder (FSAD). These were secondary efficacy endpoints in this Phase 2b study of Sildenafil Cream 3.6% for the treatment of FSAD in healthy premenopausal women described in Example 2.

Arousal Diary (SSE): To evaluate the efficacy of Sildenafil Cream, 3.6% in patients with FSAD as measured by change from baseline to Week 4 (Visit 5) and Week 8 (Visit 6) in number of Satisfactory Sexual Events (question #11 in the Arousal Diary) completed by daily prompt.

Following a baseline single-blind placebo run-in period, participants were randomized and used investigational product at home for 12 weeks. Participants completed 28-day recall assessments at each monthly visit. Between monthly, in-clinic assessments, participants answered 12 questions in an electronic diary (eDiary) within 24-hours of each sexual experience. Sexual experiences were categorized as “Partnered” versus “By Myself” and “Satisfactory” versus “Not Satisfactory”. Question 11 (Q11) of the eDiary asked “Were conditions appropriate for a satisfactory sexual activity (enough time, no distractions, etc.)?” The Orgasm domain (questions 22-24) of the Sexual Function Questionnaire (SFQ28) was an exploratory efficacy endpoint. Unpartnered women and women with sexual partners who did not sign informed consent were enrolled in the study. In the latter case, study product was only used in solo, unpartnered sexual experiences.

A Post-hoc analysis of sexual experiences showed that Sildenafil Cream, 3.6% users had more sexual experiences and more unpartnered sexual experiences during the double-blind treatment period compared to Placebo Cream users (Table 24). Sildenafil Cream users were significantly more likely to engage in sexual experiences even when conditions were not appropriate for a satisfactory experience (Table 24). Although the change from baseline in the mean number of SSEs at week 12 decreased during solo, unpartnered sexual experiences for both product groups (−0.12±0.27 for Sildenafil Cream versus −0.04±0.32 for Placebo, p=0.85), during the double-blind treatment period, Sildenafil Cream users reported significantly higher proportions of SSEs during unpartnered sexual experiences (197/261, 75.5%) compared to Placebo users (116/187, 62.0%) (p=0.002). Finally, unpartnered women (n=19) and women with unenrolled sexual partners (n=12), who, per protocol, had only solo sexual experiences, reported an increase from baseline in their SFQ Orgasm Domain at week 12 with Sildenafil Cream, 3.6% use (2.39±0.95) compared to Placebo users in this subset, who had decreases in this score (−0.19±0.75) (p=0.06).

TABLE 24
Sexual Experiences by Category, Visit and Product
Use during the Double-Blind Treatment Period

| | Sildenafil Cream 3.6% | | Placebo Cream | | P |
|---|---|---|---|---|---|
| Sexual Experience Category | N/Total | % Group | N/Total | % Group | value |
| Total Sexual Experiences | 1131 | NA | 951 | NA | 0.87 |
| During Double-Blind | | | | | |
| Treatment Period | | | | | |
| Total Unpartnered Sexual | 261/1131 | 23.1% | 187/951 | 19.7% | 0.06 |
| Experiences During Double- | | | | | |
| Blind Treatment | | | | | |
| Q11 = NO Conditions not | 59/1131 | 5.2% | 25/951 | 2.6% | 0.003 |
| Appropriate for Satisfactory | | | | | |
| Sexual Experience | | | | | |

These data support that Sildenafil Cream, 3.6% enhanced solo, unpartnered sexual experiences, which represented approximately 1 in 5 sexual events in this 12-week FSAD treatment study.

### Example 8: Female Sexual Distress in an Exploratory, Phase 2b, Randomized, Double-Blind, Placebo-Controlled Clinical Trial of Sildenafil Cream, 3.6% for the Treatment of Female Sexual Arousal Disorder

The diagnosis of female sexual arousal disorder (FSAD) requires that individuals experience clinically significant distress from their symptoms. Pre-trial content validity investigations were conducted to understand how women with FSAD best described their distress associated with this diagnosis.

This example describes post-hoc analysis of responses to the Female Sexual Distress Scale-Desire, Arousal, Orgasm (FSDS-DAO) survey among healthy premenopausal women enrolled in a Phase 2b, exploratory, randomized, placebo-controlled, double-blind study of Sildenafil Cream, 3.6% for FSAD treatment described in Example 2.

After informed consent, the primary and secondary (if applicable) enrollment diagnoses for each participant were established during a one-on-one clinical interview with independent experts. Baseline assessments were measured after a one-month single-blind placebo run-in period. Participants were then randomized to Sildenafil Cream, 3.6% (n=101) versus Placebo Cream (n=99) and used the study product as needed at home for 12 weeks, with monthly in-clinic assessments. The change from baseline at week 12 in the arousal sensation (AS) domain of the sexual function questionnaire (SFQ28) and question 14 (Q14) of the FSDS-DAO were co-primary efficacy endpoints. All 15 FSDS-DAO questions begin with “How often did you feel, in the past 30 days” with a Likert score of 0 (Never) to 4 (Always). FSDS-DAO responses were analyzed based on the entire intent-to-treat (ITT) population and in a post-hoc analysis, based on female sexual dysfunction (FSD) diagnoses at enrollment.

FSDS-DAO (Q14)—28 Day Recall: To evaluate the efficacy of Sildenafil Cream, 3.6% vs Placebo Cream in patients with FSAD as measured by change from baseline to end of study in the score for feeling concerned by difficulties with sexual arousal-Item #14 only of the Female Sexual Distress Scale-Desire, Arousal, Orgasm (FSDS-DAO). The FSDS-DAO (Q14) score at the end of the Single-Blind Placebo Run-In will serve as baseline and the Sildenafil Cream, 3.6% arm results was compared with the Placebo Cream arm results.

Changes from baseline at week 12 in the co-primary efficacy endpoint of FSDS-DAO questions, showed reduced sexual distress among Sildenafil Cream, 3.6% users versus Placebo Cream users (Table 24) in this subset population. Questions 3, 5, 6, 8 and 10 queried participants about generalized sexual distress, using language more congruent with pre-trial content validity investigations and post-trial psychometric validity analyses.

TABLE 25
Change from Baseline at Week 12 in FSDS-DAO Questions
among Participants with FSAD Only or FSAD with HSDD

| | Sildenafil Cream 3.6% (n = 34) | | | | |
|---|---|---|---|---|---|
| | Least Squares | Standard | Placebo Cream (n = 27) | | |
| | (LS) Mean | Error | LS Mean | | P |
| FSDS-DAO Question | Change | (SE) | Change | SE | value |
| Q3 “Guilty about Sexual | −0.64 | 0.18 | −0.09 | 0.20 | 0.04 |
| Difficulties” | | | | | |
| Q5 “Stressed about Sex” | −0.54 | 0.17 | 0.02 | 0.19 | 0.03 |
| Q6 “Inferior because of Sexual | −0.40 | 0.19 | 0.03 | 0.21 | 0.13 |
| Problems” | | | | | |
| Q8 “Sexually Inadequate” | −0.48 | 0.17 | 0.04 | 0.19 | 0.05 |
| Q10 “Embarrassed about Sexual | −0.59 | 0.17 | 0.01 | 0.19 | 0.03 |
| Problems” | | | | | |
| Q14 “Concerned about | −0.21 | 0.16 | −0.22 | 0.16 | 0.95 |
| Difficulties with Sexual | | | | | |
| Arousal” | | | | | |

These data support that a subset of the ITT population, women with a diagnosis of FSAD only or primary FSAD with secondary HSDD symptoms, experienced reduced sexual distress after receiving Sildenafil Cream, 3.6%. Data from post-trial psychometric evaluations of the FSDS-DAO support item 5 “How often in the past 30 days have you felt stressed about sex” was one of the strongest items of the FSDS-DAO for the measure of sexual distress in the ITT population.

### Example 9: Impact of Enrollment Diagnosis on Efficacy Endpoints in an Exploratory, Phase 2b, Randomized, Double-Blind, Placebo-Controlled Clinical Trial of Sildenafil Cream, 3.6% for the Treatment of Female Sexual Arousal Disorder

Women with FSAD often have co-existing female sexual dysfunction (FSD) diagnoses. In this example, a post-hoc subset analysis of the effect of FSD diagnoses at enrollment on the efficacy of Sildenafil Cream, 3.6% for treatment of FSAD in healthy premenopausal women as described in Example 2 was performed.

The change from baseline at week 12 in the arousal sensation (AS) domain of the sexual function questionnaire (SFQ28) was a co-primary efficacy endpoint. Changes from baseline in the mean number of satisfactory sexual experiences (SSEs) was a secondary efficacy endpoint. Exploratory efficacy endpoints included the Desire and Orgasm domains of the SFQ28. FSD diagnoses were established during a one-on-one clinical interview with independent experts at study enrollment. A retrospective review was performed of the clinical interviews for the intent-to-treat (ITT) population who completed the study (n=174), and enrollment diagnoses were categorized.

Women randomized to Sildenafil Cream, 3.6% with FSAD as their sole FSD diagnosis (Table 26) had the largest mean increases in efficacy variables at week 12 (Table 2). Women with a primary complaint of FSAD and secondary hypoactive sexual desire disorder (HSDD) symptoms experienced substantial benefit with Sildenafil Cream, 3.6% at week 12.

TABLE 26
Diagnosis at Enrollment (ITT Population who Completed Study)

| | Sildenafil Cream, | | Placebo Cream | |
|---|---|---|---|---|
| | 3.6% (N = 90) | | (N = 84) | |
| | | % Product | | % Product |
| | N | Group | N | Group |
| FSAD Only | 5 | 5.6% | 4 | 4.8% |
| FSAD with Secondary | 28 | 31.1% | 28 | 33.3% |
| Hypoactive Sexual Desire | | | | |
| Disorder (HSDD) | | | | |
| FSAD with Female Orgasmic | 51 | 56.7% | 52 | 61.9% |
| Disorder (FOD) | | | | |
| FSAD with Dyspareunia | 6 | 6.7% | 0 | 0% |

TABLE 27
Change from Baseline at Week 12 in Efficacy Endpoints
Based on Product Assignment and Diagnosis at Enrollment

| Sildenafil Cream, 3.6% | | | | |
|---|---|---|---|---|
| Least Squares | Standard Error | Placebo Cream | | |
| (LS) Mean | (SE) in LS | LS Mean | SE in LS | |
| Change from | Mean Change | Change | Mean Change | P |
| Baseline (BL) | from BL | from BL | from BL | value |

SFQ28 Arousal Sensation Domain

| | | | | | |
|---|---|---|---|---|---|
| FSAD Only | 5.67 | 2.91 | −3.09 | 4.16 | 0.10 |
| FSAD with 2° | 2.03 | 0.62 | 0.08 | 0.71 | 0.04 |
| HSDD | | | | | |

SFQ28 Desire Domain

| | | | | | |
|---|---|---|---|---|---|
| FSAD Only | 3.06 | 1.80 | −1.97 | 2.23 | 0.13 |
| FSAD with 2° | 1.27 | 0.76 | −0.89 | 0.86 | 0.06 |
| HSDD | | | | | |

SFQ28 Orgasm Domain

| | | | | | |
|---|---|---|---|---|---|
| FSAD Only | 2.26 | 0.98 | −0.62 | 1.02 | 0.08 |
| FSAD with 2° | 1.12 | 0.49 | 0.186 | 0.52 | 0.19 |
| HSDD | | | | | |

These data support that future target populations, including healthy premenopausal women with FSAD only or a primary complaint of FSAD with secondary HSDD symptoms, will likely benefit from Sildenafil Cream, 3.6% treatment.

### Example 10: Preliminary Efficacy of Sildenafil Cream, 3.6% in an Exploratory, Phase 2b, Randomized, Double-Blind, Placebo-Controlled Clinical Trial of for the Treatment of Female Sexual Arousal Disorder

In this example, co-primary efficacy endpoints including the change from baseline at week 12 (end of treatment) in (1) the arousal sensation (AS) domain of the sexual function questionnaire (SFQ28) and (2) question 14 (Q14) of the female sexual distress scale-desire, arousal, orgasm (FSDS-DAO) were evaluated from the study as described in Example 2.

Participants categorized each sexual experience as satisfactory or not in an electronic diary within 24 hours of the event. The secondary efficacy endpoint was the change from baseline to end of study in the number and proportion of Satisfactory Sexual Events (SSEs). Exploratory endpoints included the Desire and Orgasm domains of the SFQ28 questionnaire.

At the end of the study, exit interviews were done and data from the exit interviews were utilized in a separate psychometric validation analysis to determine meaningful, clinically relevant changes in the efficacy endpoints for the intent-to-treat (ITT) population.

TABLE 28
Schedule of Events for Efficacy Assessments

| | | | Start | Start of | | | | | |
|---|---|---|---|---|---|---|---|---|---|
| | | | of No | Single- | | | | | |
| | | | Drug | Blind | | Start of Each Month of | | | |
| Assessment | | Medical | Run- | Placebo | | Double-Blind Dosing | | | Follow-Up |
| Category | Assessments | Screening | In | Run-In | | Period | | | Assessment |
| | Visit | 1 | 2 | 3 | 4 | 5 | 6 | 7 | Phone Call |
| | Study Weeks | 0 | 1 | 5 | 9 | 13 | 17 | 21 | 22 |
| | Study Days | Day -28 | 0 | Visit | Visit | Visit | Visit | Visit | Visit |
| | | to -1 | | 2 + | 3 + | 4 + | 5 + | 6 + | 7 + |
| | | | | 28- | 28- | 28- | 28- | 28- | 4- |
| | | | | 31 | 31 | 31 | 31 | 31 | 10 |
| | | | | days | days | days | days | days | days |
| Informed | Informed Consent | X | | | | | | | |
| Consent | Inclusion/Exclusion | X^{a} | X^{a} | X | X | X | X | X | |
| | Medical History | X^{a} | | | | | | | |
| Eligibility | Clinical Interview | X | | | | | | | |
| Determination | PHQ-8 | X | X | X | X | | | | |
| | GAD-7 | X | X | X | X | | | | |
| Study | Randomization | | | | X | | | | |
| Administration | Investigational | | | X^{g} | X | X | X | | |
| | Product Dispensed | | | | | | | | |
| | Investigational | | | | X | X | X | X | |
| | Product Returned | | | | | | | | |
| | to Site | | | | | | | | |
| Efficacy | FSDS-DAO | X | | X^{h} | X | X | X | X | |
| Assessments | SFQ28 | X | | X^{h} | X | X | X | X | |
| | Arousal Diary | | X | X^{h} | X | X | X | X | |
| | PGI-S^{a} | X | | X | X | X | X | X | |
| | PGI-C^{b} | | | X | X | X | X | X | |
| | PGI-F^{c} | | | X | X | X | X | X | |
| | PGI-C Concern^{d} | | | X | X | X | X | X | |
| | PGI-C SSE^{e} | | | X | X | X | X | X | |
| | PBE^{f} | | | | | | | X | |

^{a}The Patient Global Impression of Severity (PGI-S) is a patient-reported outcome that captures the patient's assessment of severity of their condition.
^{b}The Patient Global Impression of Change (PGI-C) is a patient reported outcome that captures the patient's assessment of change in their condition.
^{c}The Patient Global Impression of Frequency (PGI-F) is patient reported outcome that captures the patient's assessment of change in the frequency of sexual arousal problems.
^{d}The Patient Global Impression Change in Concern with Sexual Difficulties (PGI-C Concern) is a patient reported outcome that captures the patient's assessment of change in concern with sexual difficulties.
^{e}The Patient Global Impression of Change in Satisfactory Sexual Events (PGI-C SSE) is a patient reported outcome that captures the patients overall changed in satisfactory sexual activity.
^{f}The Patient Benefit Evaluation (PBE) is a patient-reported outcome that assesses women's perceptions of benefit from the study medication with a single item.
^{g}All patients are dispensed Placebo Cream at Visit 3.
^{h}Responses and scores following the 4-week single blind placebo run-in will be utilized to establish baseline.

Randomization: Participants were randomized via Interactive Response Technology following the single-blind run-in period. They were randomized to either 2-gram Sildenafil Cream, 3.6% or 2-gram Placebo Cream in a 1:1 ratio. There were no allocation errors.

Study Product: Placebo Cream and Sildenafil Cream, 3.6%, were both water-based, white to off-white in appearance, and identical in color, smell, and consistency. The Sildenafil Cream contained 5% (w/w) sildenafil citrate and other excipients, resulting in a 3.6% Sildenafil Cream. The Placebo Cream contained the same ingredients as in the Sildenafil Cream, but without the active ingredient, sildenafil citrate. The IP was supplied in 30-gram, white, aluminum tubes. IP was stored at room temperature (20-25° C.) in a secure, temperature and humidity monitored area accessible only to the Investigator or designee at each study site.

Investigational Product Dosing: Each month, IP was dispensed in a 30-gram tube, along with nine 2-gram dosing cards. Participants were instructed to apply no more than one, 2-gram application of product per calendar day and no more than 9 applications per month. After dispensing a 2-gram portion on to the dosing card, participants used their index finger to apply 2 grams of the IP approximately 10-20 minutes prior to the sexual event. One gram was applied externally to the anterior labial commissure, prepuce of the clitoris, glans, frenulum, vestibule, and labia minora. The other gram was applied to the anterior distal vagina to an approximate depth 0-3 cm or about halfway between the distal and proximal interphalangeal joints, targeting the distal, lower ⅓ of the vagina. Participants were instructed to wash off the cream and have their partner wash off the cream, if relevant, after the sexual experience. Use of more than 9 applications per month was noted as a protocol violation.

Preliminary Efficacy Assessments: For the co-primary efficacy endpoints, participants answered the 28-day recall SFQ28 questionnaire and the FSDS-DAO questionnaire at each monthly visit. The AS domain of the SFQ28 questionnaire is made up of questions 6, 7, 8 and 9. All the SFQ28 questions began with “Over the last 4 weeks”. Question 6 asked “how often did you have a feeling of ‘warmth’ in your vagina/genital area when you took part in sexual activity?”. Question 7 was “in general, how much ‘warmth’ did you feel in your vagina/genital area when you took part in sexual activity?”. Question 8 queried “in general, how much ‘pulsating’ (‘tingling’) in your vagina/genital area did you notice when you took part in sexual activity?”. Question 9 asked “in general, how much ‘pulsating’ (‘tingling’) in your vagina/genital area did you notice when you took part in sexual activity?”. These 4 questions were answered with a Likert scale ranging from 1-5, with 1 being no sensation and 5 being a very strong sensation. The range of scores for the AS domain was 4-20, with higher scores indicating higher sexual function. If a participant answered “I did not take part in sexual activity” to any of the 4 AS domain questions, indicating that the responses would therefore be not applicable for the last month, then the AS domain was not calculated for that participant for that 28-day period.

The FSDS-DAO 28-day recall survey included 15 questions, each of which began with the phrase “How often in the past 30 days did you feel” with responses of 0-4, with 0 indicating never feeling this way and 4 indicating always feeling this way. Q14, the co-primary efficacy endpoint, asked “how often in the past 30 days did you feel concerned by difficulties with sexual arousal?”. The range of scores for Q14 of the FSDS-DAO was 0-4 with lower scores indicating decreased levels of sexual distress.

For the secondary efficacy endpoint, participants were given an eDiary at visit 2, the start of the no-drug run-in period. The eDiary prompted the participant every 24 hours to assess whether there had been a sexual event. If there was a sexual event, the eDiary prompted the participant to answer safety questions along with the question “Did you consider sexual activity satisfactory for you?” with answers of Yes or No. The secondary endpoint of satisfactory sexual experiences (SSEs) measured the average number of SSEs per month per product cohort and the average proportion of SSEs of total sexual experiences per month per product cohort.

The exploratory endpoints were evaluated from both 28-day recall questionnaires. The exploratory endpoints are detailed in Table 29.

TABLE 29
Exploratory Preliminary Efficacy Endpoints

| | | | | | | Score |
|---|---|---|---|---|---|---|
| | Endpoint | | | | | Suggesting |
| | Domain | | | | Domain | Normal |
| Source | Name | Questions | Question Text | Answer Scale and Text | Range | Function |
| SFQ28 | Desire | 1 | Over the last 4 | (1) Not at All | 5-31 | ≥23 |
| | | | weeks, how often | (2) Rarely (3) Sometimes | | |
| | | | have you had | (4) Often (5) Very Often | | |
| | | | pleasurable thoughts | | | |
| | | | and feelings about | | | |
| | | | sexual activity? | | | |
| | | 2 | Over the last 4 | (1) Not at All | | |
| | | | weeks, how often | (2) Rarely (3) Sometimes | | |
| | | | have you wanted to | (4) Often (5) Very Often | | |
| | | | be sensually touched | | | |
| | | | and caressed by your | | | |
| | | | partner? | | | |
| | | 3 | Over the last 4 | (1) Not at All | | |
| | | | weeks, how often | (2) Rarely (3) Sometimes | | |
| | | | have you wanted to | (4) Often (5) Very Often | | |
| | | | take part in sexual | | | |
| | | | activity? | | | |
| | | 4 | Over the last 4 | (1) Not at All | | |
| | | | weeks, how often | (2) Rarely (3) Sometimes | | |
| | | | have you initiated | (4) Often (5) Very Often | | |
| | | | sexual activity with | | | |
| | | | your partner? | | | |
| | | 14 | Over the past 4 | (0) I did not take part in | | |
| | | | weeks, how often did | sexual activity (1) | | |
| | | | you take part in | Once/twice (2) 3-4 | | |
| | | | sexual activity with | times (3) 5-8 times (4) | | |
| | | | penetration (e.g. | 9-12 times (5) 13-16 | | |
| | | | vaginal penetration | times (6) >16 times | | |
| | | | and intercourse)? | | | |
| | | 26 | Thinking about your | (1) Not at All | | |
| | | | sexual life over the | (2) Rarely (3) Sometimes | | |
| | | | last 4 weeks, how | (4) Often (5) Very Often | | |
| | | | often did you look | | | |
| | | | forward to sexual | | | |
| | | | activity? | | | |
| SFQ28 | Orgasm | 22 | Over the last 4 | (NA) I did not take part | 1-15 | ≥12 |
| | | | weeks, how often did | in sexual activity (1) Not | | |
| | | | you have an orgasm | at all (2) Sometimes (3) | | |
| | | | when you took part in | Often (4) Very Often (5) | | |
| | | | sexual activity (may | Every Time | | |
| | | | be with or without a | | | |
| | | | partner?) | | | |
| | | 23 | Over the last 4 | (0) I did not have any | | |
| | | | weeks, in general, | orgasms (1) Not | | |
| | | | how pleasurable were | Pleasurable (2) Slightly | | |
| | | | the orgasms that you | Pleasurable (3) | | |
| | | | had? | Moderately Pleasurable | | |
| | | | | (4) Very Pleasurable (5) | | |
| | | | | Extremely Pleasurable | | |
| | | 24 | Over the last 4 | (0) I did not have any | | |
| | | | weeks, in general, | orgasms (1) Very | | |
| | | | how easy was it for | Difficult (2) Quite | | |
| | | | you to reach orgasm? | Difficult (3) Neither Easy | | |
| | | | | nor Difficult (4) Quite | | |
| | | | | Easy (5) Very Easy | | |
| | | 13 | Over the last 4 | (NA) I did not take part | | |
| | | | weeks, how much | in sexual activity (1) | | |
| | | | emotional sexual | None (2) Slightly | | |
| | | | arousal did you | Aroused (3) Moderately | | |
| | | | notice when you took | Aroused (4) Very | | |
| | | | part in sexual | Aroused (5) Extremely | | |
| | | | activity? (e.g. feeling | Aroused | | |
| | | | excited, feeling | | | |
| | | | ‘turned on’, wanting | | | |
| | | | sexual activity to | | | |
| | | | continue). | | | |
| FSDSDAO | Total | 1 | Distressed about your | All Questions Start with: | 0-60 | <18 |
| | Score: | | sex life | Below is a list of feelings | | |
| | | 2 | Unhappy about your | and problems that women | | |
| | | | sexual relationship | sometimes have | | |
| | | 3 | Guilty about sexual | concerning their | | |
| | | | difficulties | sexuality. Please read | | |
| | | 4 | Frustrated by your | each item carefully and | | |
| | | | sexual problems | circle the number that | | |
| | | 5 | Stressed about sex | describes “How often that | | |
| | | 6 | Inferior because of | problem has bothered you | | |
| | | | sexual problems | or caused you distress | | |
| | | 7 | Worried about sex | during the past 30 days | | |
| | | 8 | Sexually inadequate | including today” | | |
| | | 9 | Regrets about your | Answers are (0) Never | | |
| | | | sexual functioning | (1) Rarely (2) | | |
| | | 10 | Embarrassed about | Occasionally (3) | | |
| | | | sexual problems | Frequently (4) Always | | |
| | | 11 | Dissatisfied with | | | |
| | | | your sex life | | | |
| | | 12 | Angry about your sex | | | |
| | | | life | | | |
| | | 13 | Bothered by low | | | |
| | | | sexual desire | | | |
| | | 14 | Concerned by | | | |
| | | | difficulties with | | | |
| | | | sexual arousal | | | |
| | | 15 | Frustrated by | | | |
| | | | problems with | | | |
| | | | orgasm | | | |
| | | 8 | Were you satisfied | (1) Very Dissatisfied (2) | | |
| | | | with your genital | Dissatisfied (3) Neither | | |
| | | | arousal during sexual | satisfied nor dissatisfied | | |
| | | | activity? | (4) Satisfied (5) Very | | |
| | | | | Satisfied | | |
| | | 9 | Were you frustrated | (1) Extremely frustrated | | |
| | | | with your genital | (2) Very frustrated (3) | | |
| | | | arousal during sexual | Moderately frustrated (4) | | |
| | | | activity? | Slightly frustrated (5) Not | | |
| | | | | at all Frustrated | | |
| | | 10 | Were you concerned | (1) Extremely concerned | | |
| | | | about difficulties | (2) Very concerned (3) | | |
| | | | with your sexual | Moderately concerned (4) | | |
| | | | arousal? | Slightly concerned (5) | | |
| | | | | Not at all Concerned | | |

Statistical Analysis: With a dropout rate of approximately 13%, given the slower than anticipated enrollment, the protocol randomized 169 to 175 patients to ensure that a minimum of 150 patients were to complete the study.

An additional n=30 patients to the planned enrollment were included for a pre-planned analysis to investigate whether completion of the eDiary questions influenced patient SFQ28 AS domain scores and Q14 of the FSDS-DAO. These additional 30 patients completed the entire trial but did not complete the eDiary throughout the trial. These patients did not affect the primary study objectives as they were not included in the analysis of the co-primary or exploratory endpoints.

Baseline was defined as the end of the single-blind placebo run-in (visit 4) for efficacy endpoints. End of study was defined as the end of the double-blind dosing period for efficacy evaluations at visit 7 or week 12 of treatment. The analysis was conducted using a mixed effect model repeated measure (MMRM) which handles missing data via maximum likelihood under the assumption of missing at random. Missing at random assumes that those who have missing values would have continued just like those (with the same history of values) without missing values and is a “continuing on treatment” question.

The individual clinical interviews for the 174 participants in the ITT population who completed all study visits were reviewed. The participants' main FSD diagnosis and accompanying secondary FSD diagnoses, if applicable, were categorized based on data from the enrollment clinical interview. The efficacy variables were then calculated in a post-hoc analysis by product, visit, and FSD enrollment diagnosis.

All statistical analyses used SAS version 9.4 or higher. Unless otherwise noted, categorical variables were summarized using counts and percentages. Continuous variables were summarized using the number of observations (n), mean, Standard Deviation (STD), median, first quartile (Q1), third quartile (Q3), minimum and maximum. The primary analysis of the SFQ28 (AS) and the FSDS-DAO (Q14) included a 2-sided test with a significant level of 0.05.

Results: Volunteers (n=833) and their sexual partners (n=605) underwent informed consent. Of the consented subjects, 277 eligible participants entered the no-drug run-in period and 252 entered a 28-day single-blind placebo run-in period to establish baseline sexual function and exclude women with a predefined placebo response. Two hundred participants were randomized to Sildenafil Cream, 3.6% (n=101) versus Placebo Cream (n=99) and 99 Sildenafil assigned women and 94 Placebo assigned women received at least one dose of their assigned IP and comprise the ITT population. There were no allocation errors. A total of 174 women (90 Sildenafil and 84 Placebo) completed all study visits. Table 30 displays the enrollment diagnoses, demographics, and baseline characteristics of subjects and their sexual partners, if applicable, included in the safety analysis population. Most participants were in their mid-thirties, non-Hispanic White, and had a male sexual partner.

TABLE 30
Demographic and Baseline Characteristics of Participants and Sexual Partners
(Safety Population)

| Participant | Participants Sildenafil | | Participants Placebo | | Participants Total | |
|---|---|---|---|---|---|---|
| Characteristic | (N = 99) | | (N = 134) | | (N = 233) | |
| Participant | Mean | SD | Mean | SD | Mean | SD |
| Continuous | | | | | | |
| Variables | | | | | | |
| Age (Years) | 36.3 | 7.14 | 36.3 | 7.21 | 36.3 | 7.17 |
| Baseline Body | 27.1 | 4.7 | 27.2 | 4.6 | 27.2 | 4.6 |
| Mass Index (BMI) | | | | | | |
| (kg/m²) | | | | | | |
| Participant | N | % Total | N | % Total | N | % Total Safety |
| Categorical | | Product | | Product | | Population |
| Variables | | Group | | Group | | |

Enrollment Diagnosis of 174 participants (90 Sildenafil Cream 3.6% and 84 Placebo Cream)
who completed all visits

| FSAD Only | 5 | 5.6% | 4 | 4.8% | 9 | 5.2% |
|---|---|---|---|---|---|---|
| FSAD with | 28 | 31.1% | 28 | 31.1% | 56 | 32.2% |
| Hypoactive Sexual | | | | | | |
| Desire Disorder | | | | | | |
| (HSDD) | | | | | | |
| FSAD with Female | ₅1 | 56.7% | ₅2 | 61.9% | 103 | 59.2% |
| Orgasm Disorder | | | | | | |
| (FO_{D}) | | | | | | |
| FSAD with pain | 6 | 6.7% | 0 | 0% | 6 | 3.4% |
| Gender Identity | | | | | | |
| Cis Gender Female | 99 | 100 | 134 | 100 | 233 | 100 |
| Ethnicity | | | | | | |
| Hispanic or Latino | 14 | 14.1 | 24 | 17.9 | 38 | 16.3 |
| Not Hispanic or | 85 | 85.9 | 110 | 82.1 | 195 | 83.7 |
| Latino | | | | | | |
| Race | | | | | | |
| American Indian or | 0 | 0 | 2 | 1.5 | 2 | 0.9 |
| Alaskan Native | | | | | | |
| Asian | 5 | 5.1 | 7 | 5.2 | 12 | 5.2 |
| Black or African | 6 | 6.1 | 14 | 10.4 | 20 | 8.6 |
| American | | | | | | |
| Native Hawaiian or | 1 | 1.0 | 0 | 0 | 1 | 0.4 |
| other Pacific | | | | | | |
| Islander | | | | | | |
| White | 84 | 84.8 | 110 | 82.1 | 194 | 83.3 |
| Mixed Race or | 3 | 3.0 | 1 | 0.7 | 4 | 1.7 |
| Other | | | | | | |
| Employment Status | | | | | | |
| Employed Full- | 73 | 73.7 | 97 | 72.4 | 170 | 73.0 |
| Time | | | | | | |
| Employed Part- | 6 | 6.1 | 7 | 5.2 | 13 | 5.6 |
| Time | | | | | | |
| Self-Employed | 5 | 5.1 | 4 | 3.0 | 9 | 3.9 |
| Student | 1 | 1.0 | 4 | 3.0 | 5 | 2.1 |
| Retired | 0 | 0 | 1 | 0.7 | 1 | 0.4 |
| Unemployed | 14 | 14.1 | 21 | 15.7 | 35 | 15.0 |
| Highest Level of | | | | | | |
| Education | | | | | | |
| Some High School | 5 | 5.1 | 10 | 7.5 | 15 | 6.4 |
| or High School | | | | | | |
| Diploma/Certificate | | | | | | |
| Technical School, | 24 | 24.2 | 37 | 27.6 | 61 | 26.2 |
| Occupational | | | | | | |
| School, Associates | | | | | | |
| Degree and or | | | | | | |
| Some College | | | | | | |
| Bachelor's Degree | 40 | 40.4 | 43 | 32.1 | 83 | 35.6 |
| Master's or | 6 | 6.1 | 11 | 8.2 | 17 | 7.3 |
| Doctoral Degree | | | | | | |
| Missing | 0 | 0 | 1 | 0.7 | 1 | 0.4 |
| Partner | Partners Sildenafil | | Partners Placebo | | Partners Total | |
| Characteristic | (N = 91) | | (N = 112) | | (N = 203) | |
| Partner Continuous | Mean | SD | Mean | SD | Mean | SD |
| Variables | | | | | | |
| Age (Years) | 38.5 | 8.60 | 37.6 | 8.48 | 38.0 | 8.52 |
| BMI (kg/m²) | 28.5 | 5.7 | 28.7 | 4.5 | 28.6 | 5.1 |
| Partner Categorical | N | % Total | N | % Total | N | % Total Study |
| Variables | | Product | | Product | | Safety |
| | | Group | | Group | | Population |
| Sex | | | | | | |
| Female | 5 | 5.5 | 10 | 8.9 | 15 | 7.4 |
| Male | 86 | 94.5 | 102 | 91.1 | 188 | 92.6 |

Table 31 displays the preliminary efficacy results at baseline (visit 4) and during the double-blind dosing period (visits 5-7) based on product assignment for the ITT population. No statistically significant improvement in either co-primary efficacy endpoint during the double-blind dosing period (all p values>0.05) were observed. The largest improvement in the SFQ28 AS domain (co-primary endpoint) occurred at week 12 among Sildenafil Cream users (LS mean change from baseline=1.1) but this was not statistically significant (p=0.60).

There were no statistically significant differences in the mean number of sexual experiences graded as “satisfactory” in the eDiary between Sildenafil and Placebo Cream users at any point during the double-blind dosing period (all p values>0.32). The mean proportion of SSEs was also similar between the two dosing cohorts (all p values>0.15). The only significant change noted for the ITT population was a statistically significant increase in the SFQ28 Desire domain scores at week 8 for Sildenafil Cream, 3.6% users (p=0.04, Table 31).

TABLE 31
Preliminary Efficacy Endpoints for Intent-to-Treat Population

| | Sildenafil | | | | | Placebo | | | | | | |
|---|---|---|---|---|---|---|---|---|---|---|---|---|
| | | | | LS | | | | | LS | | Sildenafil- | |
| Objective | | | | Mean | | | | | Mean | | Placebo | |
| Endpoint | | | | Change | SE LS | | | | Change | SE LS | LS Mean | |
| Time | N | Mean | SD | BL | Mean | N | Mean | SD | BL | Mean | Diff | P |

1º SFQ28 Arousal Sensation (AS) Domain

| | | | | | | | | | | | | |
|---|---|---|---|---|---|---|---|---|---|---|---|---|
| BL | 84 | 8.0 | 2.80 | NA | NA | 72 | 7.7 | 2.84 | NA | NA | | NA |
| Week 4 | 77 | 8.4 | 2.65 | 0.4 | 0.29 | 67 | 8.5 | 3.31 | 0.8 | 0.31 | −0.4 | 0.38 |
| Week 8 | 73 | 8.6 | 3.33 | 0.7 | 0.32 | 63 | 8.4 | 3.30 | 0.7 | 3.17 | −0.1 | 0.90 |
| Week 12 | 69 | 9.1 | 3.58 | 1.1 | 0.39 | 59 | 8.6 | 3.59 | 0.8 | 0.42 | 0.3 | 0.60 |

1º Female Sexual Distress Scale - Desire Arousal Orgasm (FSDS-DAO) Question 14 (Q14)

| | | | | | | | | | | | | |
|---|---|---|---|---|---|---|---|---|---|---|---|---|
| BL | 84 | 2.5 | 0.95 | NA | NA | 73 | 2.6 | 1.05 | NA | NA | | NA |
| Week 4 | 78 | 2.4 | 1.02 | −0.1 | 0.09 | 70 | 2.6 | 0.91 | 0 | 0.1 | −0.1 | 0.39 |
| Week 8 | 73 | 2.3 | 1.07 | −0.2 | 0.1 | 64 | 2.5 | 0.91 | −0.1 | 0.11 | −0.1 | 0.35 |
| Week 12 | 71 | 2.3 | 1.06 | −0.2 | 0.11 | 61 | 2.4 | 1.02 | −0.2 | 0.12 | 0 | 0.84 |

2º Mean Number of Satisfactory Sexual Events (SSEs) per month

| | | | | | | | | | | | | |
|---|---|---|---|---|---|---|---|---|---|---|---|---|
| BL | 80 | 2.9 | 2.86 | NA | NA | 69 | 2.8 | 2.76 | NA | NA | NA | NA |
| Week 4 | 81 | 3.2 | 2.93 | 0.2 | 0.22 | 73 | 3.2 | 2.88 | 0.5 | 0.24 | −0.2 | 0.47 |
| Week 8 | 77 | 3.1 | 2.56 | 0.2 | 0.21 | 66 | 2.8 | 2.56 | −0.1 | 0.23 | 0.3 | 0.32 |
| Week 12 | 72 | 3.1 | 2.75 | 0.1 | 0.25 | 65 | 2.8 | 2.57 | −0.1 | 0.27 | 0.1 | 0.73 |

2° Mean Proportion (SSEs/Total Sexual Events) per month

| | | | | | | | | | | | | |
|---|---|---|---|---|---|---|---|---|---|---|---|---|
| BL | 80 | 0.6 | 0.41 | NA | NA | 69 | 0.5 | 0.37 | NA | NA | NA | NA |
| Week 4 | 81 | 0.6 | 0.37 | 0.1 | 0.03 | 73 | 0.6 | 0.37 | 0.1 | 0.04 | 0 | 0.43 |
| Week 8 | 77 | 0.6 | 0.38 | 0.1 | 0.03 | 66 | 0.6 | 0.38 | 0.1 | 0.04 | 0 | 0.80 |
| Week 12 | 72 | 0.6 | 0.39 | 0.1 | 0.04 | 65 | 0.7 | 0.39 | 0.1 | 0.04 | −0.1 | 0.15 |

Exploratory SFQ28 Desire Domain

| | | | | | | | | | | | | |
|---|---|---|---|---|---|---|---|---|---|---|---|---|
| BL | 84 | 16.1 | 4.32 | NA | NA | 72 | 16.6 | 4.31 | NA | NA | NA | NA |
| Week 4 | 78 | 16.7 | 3.93 | 0.5 | 0.34 | 69 | 17 | 4.61 | 0.7 | 0.37 | −0.2 | 0.69 |
| Week 8 | 73 | 16.8 | 4.56 | 0.9 | 0.41 | 64 | 16 | 5.08 | −0.4 | 0.44 | 1.3 | 0.04 |
| Week 12 | 70 | 16.7 | 4.86 | 0.8 | 0.45 | 60 | 16.5 | 4.64 | 0 | 0.49 | 0.8 | 0.24 |

When the efficacy endpoints were compared based on enrollment FSD diagnosis, women with FSAD and symptoms of FOD did not experience any substantial treatment benefit at week 12 from Sildenafil Cream, 3.6% use (data not shown, all p values>0.22). Women with FSAD as their sole diagnosis, although uncommon, experienced the largest improvements in sexual function at week 12 with Sildenafil Cream, 3.6% (Table 32), which were not statistically significant, likely due to the small sample size. When participants with FSAD and FOD symptoms and FSAD with pain were removed from the analysis, a subset of participants (Sildenafil Cream, 3.6%=33 and Placebo Cream=32) remained. This group of participants, who had FSAD as their primary complaint, but also reported secondary symptoms of HSDD, experienced significant improvements with Sildenafil Cream, 3.6% use at week 12 in the co-primary endpoint of SFQ28 AS domain ((LS Mean [SE] 2.03 [0.62]) compared to the Placebo Cream group (LS Mean [SE] 0.08 [0.71]), p=0.04, Table 32) and had large improvements in the SFQ28 Desire Domain (1.27 [0.76], p=0.06, Table 32), and an over six-fold increase compared to Placebo Cream users in the SFQ28 Orgasm domain at week 12 (1.12 [0.49], p=0.19, Table 32) at Week 12 compared with women with the same diagnoses in the Placebo Cream group (−0.89 [0.86]) for Desire (p=0.06) and (0.18 [0.52]) for Orgasm Domain (p=0.19).

Although the subset population of women randomized to Sildenafil Cream, 3.6% with either FSAD only or FSAD with secondary HSDD did not demonstrate decreased sexual distress as measured by the co-primary endpoint of FSDS-DAO Q14 (p=0.95, Table 32), which assesses concern about difficulties with sexual arousal, it was found that 5 other FSDS-DAO questions, which asked about generalized feelings related to sexual distress, showed either significant improvement (Questions 3, 5 and 10, all p values≤0.04, Table 32) or greater improvement with Sildenafil Cream, 3.6% compared to Placebo Cream (Question 6, p=0.23 and Question 8, p=0.14, Table 32). Participants with FSAD and secondary female orgasmic disorder (FOD) achieved no meaningful benefit with Sildenafil Cream compared to Placebo Cream (all p values>0.22).

TABLE 32
Subset Population Results based on Enrollment Female Sexual Dysfunction
Diagnoses, Product Use and Visit

| | Sildenafil | | Placebo | | | | |
|---|---|---|---|---|---|---|---|
| | | Standard | | Standard | | | |
| | Least | Error | Least | Error | | | |
| | Squares | (SE) in | Squares | (SE) in | Sildenafil - Placebo | | |
| | (LS) | LS | (LS) | LS | Difference | | |
| | Mean | Mean | Mean | Mean | in LS | SE in | |
| | Change | Change | Change | Change | Mean | Difference | |
| Variable and Subset | from | from | from | from | (Sildenafil- | of LS | |
| Population | Baseline | Baseline | Baseline | Baseline | Placebo) | Mean | P value |

Change from Baseline at Week 12, Primary Endpoint SFQ28 AS Domain

| | | | | | | | |
|---|---|---|---|---|---|---|---|
| FSAD Only | 5.67 | 2.91 | −3.09 | 4.16 | 8.77 | 5.06 | 0.10 |
| FSAD Only and FSAD with | 2.03 | 0.62 | 0.08 | 0.71 | 1.96 | 0.95 | 0.04 |
| HSDD | | | | | | | |

Change from Baseline to Week 12, Exploratory Endpoint Desire Domain

| | | | | | | | |
|---|---|---|---|---|---|---|---|
| FSAD Only | 3.06 | 1.80 | −1.97 | 2.23 | 5.03 | 2.89 | 0.13 |
| FSAD Only and FSAD with | 1.27 | 0.76 | −0.89 | 0.86 | 2.17 | 1.14 | 0.06 |
| HSDD | | | | | | | |

Change from Baseline to Week 12, Exploratory Endpoint, Orgasm Domain

| | | | | | | | |
|---|---|---|---|---|---|---|---|
| FSAD Only | 2.26 | 0.98 | −0.62 | 1.02 | 2.88 | 1.45 | 0.08 |
| FSAD Only and FSAD with | 1.12 | 0.49 | 0.18 | 0.52 | 0.94 | 0.71 | 0.19 |
| HSDD | | | | | | | |

Change from Baseline to Week 12, Exploratory Endpoint, FSDS-DAO Questions for FSAD Only and FSAD with
HSDD groups.

| | | | | | | | |
|---|---|---|---|---|---|---|---|
| Question 3 “Guilty about | −0.73 | 0.16 | −0.23 | 0.17 | −0.50 | 0.24 | 0.04 |
| Sexual Difficulties” | | | | | | | |
| Question 5 “Stressed about | −0.50 | 0.16 | −0.02 | 0.16 | −0.49 | 0.23 | 0.04 |
| Sex” | | | | | | | |
| Question 6 “Inferior because | −0.37 | 0.17 | −0.06 | 0.18 | −0.31 | 0.26 | 0.23 |
| of Sexual Problems” | | | | | | | |
| Question 8 “Sexually | −0.40 | 0.16 | −0.06 | 0.16 | −0.34 | 0.23 | 0.14 |
| Inadequate” | | | | | | | |
| Question 10 “Embarrassed | −0.51 | 0.17 | 0.00 | 0.17 | −0.51 | 0.24 | 0.04 |
| about Sexual Problems” | | | | | | | |
| Question 14 “Concerned | −0.21 | 0.16 | −0.22 | 0.16 | 0.01 | 0.23 | 0.95 |
| about Difficulties with | | | | | | | |
| Sexual Arousal” | | | | | | | |

Discussion: Topical Sildenafil Cream, 3.6%, used over 12 weeks among healthy premenopausal women with a primary complaint of FSAD, as defined by the DSM-IV-TR guidelines, did not show significant improvement over Placebo Cream in the co-primary (SFQ28 AS domain, FSDS-DAO survey Question 14) or secondary (mean number and mean proportion of SSEs) endpoints among the ITT population during this exploratory Phase 2b study. The only efficacy endpoint which reached statistical significance in the ITT population was a significant improvement in the exploratory endpoint of the SFQ28 Desire domain at week 8 among Sildenafil Cream, 3.6% users (p=0.04), which did not persist at week 12, the end of treatment.

In this study, the participants' primary FSD complaint was related to arousal, as the mechanism of action of topical sildenafil is to augment genital tissue blood flow to treat FSAD. However, not surprisingly, it was observed that primary FSAD was often accompanied by secondary FSD diagnoses. FSAD as the primary complaint with secondary HSDD was a common diagnosis among participants. When the efficacy endpoints were compared among a subset of women with either FSAD alone or primary FSAD with secondary HSDD symptoms, either trends or significant improvements in sexual functioning with Sildenafil Cream, 3.6% as compared to Placebo were found. Significant reductions in several measures of sexual distress among this subset population of women with either FSAD alone or primary FSAD with secondary HSDD symptoms who were randomized to Sildenafil Cream, 3.6% were also observed.

FSAD as the sole FSD diagnosis was less common, but given the mechanism of action of sildenafil, it is consistent with the pharmacodynamics of the product that these select women would have the largest increase in arousal sensations with Sildenafil Cream, 3.6% use. Consistent with the finding that primary FSAD leads to other sexual problems, it was observed that woman with FSAD alone and those with FSAD and secondary HSDD also experienced increases in sexual desire with Sildenafil Cream, 3.6% use.

Participants with primary FSAD with secondary orgasmic problems derived no benefit from Sildenafil Cream, 3.6% use. Women with FSAD and secondary FOD did not benefit from Sildenafil Cream, 3.6% because FOD and anorgasmia are often associated with neurologic problems or comorbid medical conditions that would not be treated by the mechanism of action of sildenafil (i.e., increased blood flow to the genital tissue). Therefore, these women will be excluded in future studies.

The enrollment diagnoses were established by one-on-one interviews with independent experts in the field and used the DSM-IV-TR as a diagnostic source, along with other IRB approved screening forms. From review of the interviews, it was observed that FSAD as a sole diagnosis was uncommon and benefit of Sildenafil Cream, 3.6% was observed among those with desire issues, but not with orgasm problems.

Post-trial psychometric evaluations found that a 1.5-point increase in the SFQ28 AS domain was a clinically meaningful change. Subset analysis showed that women using Sildenafil Cream, 3.6% with FSAD as their sole diagnosis had an over 5-point increase in their SFQ28 AS Domain at week 12 compared to a 3-point decrease for Placebo Cream users. When the subset of Sildenafil Cream, 3.6% users with FSAD only or primary FSAD with secondary HSDD were analyzed, improvements in the SFQ28 AS, Desire, and Orgasm domains at week 12 (including a 2-point increase in the SFQ28 AS domain) were observed, while Placebo Cream users in this subset showed either decreases in these domains or increases which were a fraction of that reported with Sildenafil Cream, 3.6% use.

The US FDA generally requires that studies of sexual dysfunction treatments also measure the distress caused by the symptoms. In either the ITT or the subset populations of women with either FSAD alone or primary FSAD with secondary HSDD symptoms, significant decreases in sexual distress were observed, as measured by other FSDS-DAO questions, among the subset population. Post-trial psychometric evaluations identified FSDS-DAO question 5, which asked about general stress regarding sex, as one of the strongest items to measure sexual distress in the ITT population.

The subset population of women with either FSAD alone or primary FSAD with secondary HSDD symptoms not only demonstrated improvements in sexual function, as measured by the validated SFQ28 questionnaire, but also showed significant decreases in sexual distress, measured by the FSDS-DAO survey. Thus, both efficacy goals were supported with Sildenafil Cream, 3.6% use, in a smaller, more well-defined subset population of women with either FSAD alone or primary FSAD with secondary HSDD symptoms.

A 2-point increase in the SFQ28 domains and a 0.5-point decrease in Q14 of the FSDS-DAO was observed among the subset population of women with FSAD or FSAD and secondary HSDD.

In conclusion, topical Sildenafil Cream, 3.6% improved arousal sensation, desire, orgasm, as well as stress, guilt, and embarrassment about their sexual dysfunction, in a subset population of healthy premenopausal women with FSAD as their sole diagnosis or healthy premenopausal women with primary FSAD and secondary HSDD.

1. A method for treating Female Sexual Arousal Disorder (FSAD) or symptom thereof, comprising applying to a genital tissue of a female subject a topical composition comprising a phosphodiesterase type 5 inhibitor and/or a salt thereof.

2. The method of claim 1, wherein treating FSAD comprises increasing satisfactory sexual events, improving arousal sensation, increasing arousal lubrication, increasing orgasm, decreasing concern with sexual arousal, decreasing pain or discomfort during sexual activity, decreasing pain or discomfort after sexual activity, and/or improving satisfaction with sexual activity.

3-25. (canceled)

26. The method of claim 1, wherein the phosphodiesterase type 5 inhibitor and/or salt thereof is administered at least twice over a period of time.

27-32. (canceled)

33. The method of claim 1, wherein the phosphodiesterase type 5 inhibitor is sildenafil, avanafil, lodenafil, mirodenafil, tadalafil, vardenafil, udenafil, acetildenafil, or thiomethisosildenafil.

34. The method of claim 33, wherein the phosphodiesterase type 5 inhibitor is sildenafil.

35. The method of claim 1, wherein the topical composition further comprises:
a. a stabilization polymer;
b. propylene glycol;
c. a polysorbate surfactant;
d. L-arginine or L-arginine salt; and
e. an ionic salt.

36. The method of claim 1, wherein the composition is a cream, gel or lotion.

37-53. (canceled)

54. The method of claim 1, wherein the composition comprises each of the following compounds at concentrations of no more than ±20% of the stated concentrations:
a. purified water at a concentration of about 35% to about 55% by weight;
b. potassium chloride at a concentration of about 2.5% to about 15% by weight;
c. L-Arginine HCl at a concentration of about 2.5% to about 15% by weight;
d. glyceryl stearate at a concentration of about 4% to about 10% by weight;
e. cetyl alcohol at a concentration of about 4% to about 10% by weight;
f. squalane at a concentration of about 1% to about 8% by weight;
g. xanthan gum at a concentration of about 0.1% to about 5% by weight;
h. isopropyl myristate at a concentration of about 0.1% to about 5% by weight;
i. oleic acid at a concentration of about 0.1% to about 5% by weight;
j. propylene glycol at a concentration of about 1% to about 10% by weight;
k. polysorbate 20 at a concentration of about 0.2% to about 5% by weight; and
l. sildenafil citrate at a concentration of about 1% to about 10% by weight.

55-56. (canceled)

57. A composition comprising:
a. purified water at a concentration of about 40% by weight;
b. potassium chloride at a concentration of about 5% by weight;
c. L-Arginine HCl at a concentration of about 7.5% by weight;
d. glyceryl stearate at a concentration of about 7% by weight;
e. cetyl alcohol at a concentration of about 7% by weight;
f. squalane at a concentration of about 4% by weight;
g. xanthan gum at a concentration of about 0.8% by weight;
h. isopropyl myristate at a concentration of about 1% by weight;
i. oleic acid at a concentration of about 1% by weight;
j. propylene glycol at a concentration of about 8.5% by weight;
k. polysorbate 20 at a concentration of about 2% by weight;
l. trisodium citrate dihydrate at a concentration of about 10% by weight;
m. sodium benzoate at a concentration of about 0.2% by weight;
n. gluconolactone at a concentration of about 0.25% by weight; and
o. sildenafil citrate at a concentration of about 5% by weight.

58. A method for treating Female Sexual Arousal Disorder (FSAD) or symptom thereof, comprising:
a. selecting a female subject exhibiting one or more symptoms of FSAD, and
b. administering to a genital area of the subject a composition comprising a phosphodiesterase type 5 inhibitor and/or a salt thereof, a stabilization polymer, propylene glycol, a polysorbate surfactant, L-arginine or L-arginine salt, and an ionic salt.

59. The method of claim 58, wherein the one or more symptoms are selected from decreased genital sensitivity, lack of genital responsiveness during sexual activity, diminished genital arousal, pain and/or discomfort during sexual activity, pain and/or discomfort after sexual activity, and/or distress regarding sexual arousal difficulties.

60. The method of claim 1, wherein about 50% of the composition is applied externally to the vulva and about 50% of the composition is applied intravaginally.

61. The method of claim 60, wherein administering about 50% of the composition externally comprises:
a. applying the composition at the anterior labial commissure, prepuce of the clitoris, glans, and frenulum;
b. applying the composition to the vestibule of the vagina; and
c. applying the composition to the labia minora.

62. The method of claim 60, wherein the composition is not applied to the labia majora or pubic hair.

63. The method of claim 60, wherein administering about 50% of the composition intravaginally comprises applying the composition to the anterior distal vagina at a depth of between about 0 cm and about 3 cm in the vagina.

64. The method of claim 1, wherein the composition is administered between about 10 to about 20 minutes prior to a sexual activity.

65-87. (canceled)

88. The method of claim 1, wherein the subject exhibits one or more of the following after administration:
a. improvement in Sexual Function Questionnaire (SFQ28) arousal cognitive domain score of at least 1;
b. improvement in SFQ28 arousal lubrication domain score of at least 1;
c. improvement in SFQ28 arousal sensation domain score of at least 1.5;
d. improvement in SFQ28 desire domain score of at least 2;
e. improvement in SFQ28 orgasm domain score of at least 1.20;
f. improvement in SFQ28 pain domain score of at least 1;
g. improvement in Female Sexual Distress Scale-Desire, Arousal, Orgasm (FSDS-DAO) score of at least-7;
h. improvement in an arousal sensation score of at least 1.45;
i. improvement in genital arousal score of at least 1.13;
j. improvement in genital arousal concerns score of at least 1.49;
k. improvement in Arousal Diary Item 12-Proportion of SSEs of at least 0.25; and
l. improvement in Arousal Diary Item 12-Number of SSEs of at least 1.41.

89-94. (canceled)

95. A method of determining efficacy of a FSAD treatment, comprising:
a. obtaining a baseline score for one or more of: a Sexual Function Questionnaire (SFQ28) arousal cognitive domain score, a SFQ28 arousal lubrication domain score, a SFQ28 arousal sensation domain score, a SFQ28 desire domain score, a SFQ28 orgasm domain score, a SFQ28 pain domain score, a Female Sexual Distress Scale-Desire, Arousal, Orgasm (FSDS-DAO) score, an arousal sensation score, a genital arousal score, genital arousal concerns, Arousal Diary Item 12-Proportion of SSEs, and Arousal Diary Item 12-Number of SSEs;
b. administering the FSAD treatment to a female subject; and
c. determining improvement in one or more of the scores in step a.

96. The method of claim 95, wherein one or more of the following indicates the FSAD treatment is effective:
a. improvement in Sexual Function Questionnaire (SFQ28) arousal cognitive domain score of at least 1;
b. improvement in SFQ28 arousal lubrication domain score of at least 1;
c. improvement in SFQ28 arousal sensation domain score of at least 1.5;
d. improvement in SFQ28 desire domain score of at least 2;
e. improvement in SFQ28 orgasm domain score of at least 1.20;
f. improvement in SFQ28 pain domain score of at least 1;
g. improvement in Female Sexual Distress Scale-Desire, Arousal, Orgasm (FSDS-DAO) score of at least-7;
h. improvement in arousal sensation of at least 1.45;
i. improvement in genital arousal of at least 1.13;
j. improvement in genital arousal concerns of at least 1.49;
k. improvement in Arousal Diary Item 12-Proportion of SSEs of at least 0.25; and
l. improvement in Arousal Diary Item 12-Number of SSEs of at least 1.41.

97-98. (canceled)
